SEQUENCE LISTING (86 sequences)

SEQ ID NO: 1 - protein, 332 aa, Homo sapiens
MESSGNPESTTFFYYDLQSQPCENQAWVFATLATTVLYCLVFLLSLVGNSLVLWVLVKYESLESLTNIFILNLCLSDLVFACLLPVWISPYHWGWVLGDFLCKLLNMIFSISLYSSIFFLTIMTIHRYLSWSPLSTLRVPTLRCRVLVTMAVWVASILSSILDTIFHKVLSSGCDYSELTWYLTSVYQHNLFFLLSLGIILFCYVEILRTLFRSRSKRRHRTVKLIFAIVVAYFLSWGPYNFTLFLQTLFRTQIIRSCEAKQQLEYALLICRNLAFSHCCFNPVLYVFVGVKFRTHLKHVLRQFWFCRLQAPSPASIPHSPGAFAYEGASFY

SEQ ID NO: 2 - protein, 133 aa, Homo sapiens [sequence omitted]

SEQ ID NO: 3 - protein, 20 aa, Homo sapiens
MYRMQLLSCIALSLALVTNS

SEQ ID NO: 4 - protein, 9 aa, Artificial Sequence
  virus-derived peptide
NLVPMVATV

SEQ ID NO: 5 - protein, 9 aa, Artificial Sequence
  virus-derived peptide
VLEETSVML

SEQ ID NO: 6 - protein, 9 aa, Artificial Sequence
  virus-derived peptide
NLVPMVATV

SEQ ID NO: 7 - protein, 9 aa, Artificial Sequence
  virus-derived peptide
RIFAELEGV

SEQ ID NO: 8 - protein, 9 aa, Artificial Sequence
  virus-derived peptide
IIYTRNHEV

SEQ ID NO: 9 - protein, 9 aa, Artificial Sequence
  virus-derived peptide
VLAELVKQI

SEQ ID NO: 10 - protein, 9 aa, Artificial Sequence
  virus-derived peptide
AVGGAVASV

SEQ ID NO: 11 - protein, 9 aa, Artificial Sequence
  virus-derived peptide
TVRSHCVSK

SEQ ID NO: 12 - protein, 9 aa, Artificial Sequence
  virus-derived peptide
IMREFNSYK

SEQ ID NO: 13 - protein, 10 aa, Artificial Sequence
  virus-derived peptide
GPISHGHVLK

SEQ ID NO: 14 - protein, 9 aa, Artificial Sequence
  virus-derived peptide
ATVQGQNLK

SEQ ID NO: 15 - protein, 9 aa, Artificial Sequence
  virus-derived peptide
VYALPLKML

SEQ ID NO: 16 - protein, 10 aa, Artificial Sequence
  virus-derived peptide
AYAQKIFKIL

SEQ ID NO: 17 - protein, 9 aa, Artificial Sequence
  virus-derived peptide
QYDPVAALF

SEQ ID NO: 18 - protein, 9 aa, Artificial Sequence
  virus-derived peptide
YVKVYLESF

SEQ ID NO: 19 - protein, 9 aa, Artificial Sequence
  virus-derived peptide
DIYRIFAEL

SEQ ID NO: 20 - protein, 10 aa, Artificial Sequence
  virus-derived peptide
VFETSGGLVV

SEQ ID NO: 21 - protein, 9 aa, Artificial Sequence
  virus-derived peptide
KARDHLAVL

SEQ ID NO: 22 - protein, 9 aa, Artificial Sequence
  virus-derived peptide
QARLTVSGL

SEQ ID NO: 23 - protein, 9 aa, Artificial Sequence
  virus-derived peptide
KARAKKDEL

SEQ ID NO: 24 - protein, 9 aa, Artificial Sequence
  virus-derived peptide
QIKVRVDMV

SEQ ID NO: 25 - protein, 9 aa, Artificial Sequence
  virus-derived peptide
RRRHRQDAL

SEQ ID NO: 26 - protein, 9 aa, Artificial Sequence
  virus-derived peptide
ARVYEIKCR

SEQ ID NO: 27 - protein, 9 aa, Artificial Sequence
  virus-derived peptide
KMQVIGDQY

SEQ ID NO: 28 - protein, 9 aa, Artificial Sequence
  virus-derived peptide
NVRRSWEEL

SEQ ID NO: 29 - protein, 12 aa, Artificial Sequence
  virus-derived peptide
CPSQEPMSIYVY

SEQ ID NO: 30 - protein, 13 aa, Artificial Sequence
  virus-derived peptide
KPGKISHIMLDVA

SEQ ID NO: 31 - protein, 9 aa, Artificial Sequence
  virus-derived peptide
ELRRKMMYM

SEQ ID NO: 32 - protein, 9 aa, Artificial Sequence
  virus-derived peptide
IPSINVHHY

SEQ ID NO: 33 - protein, 9 aa, Artificial Sequence
  virus-derived peptide
FEQPTETPP

SEQ ID NO: 34 - protein, 9 aa, Artificial Sequence
  virus-derived peptide
YAYIYTTYL

SEQ ID NO: 35 - protein, 11 aa, Artificial Sequence
  virus-derived peptide
QEFFWDANDIY

SEQ ID NO: 36 - protein, 9 aa, Artificial Sequence
  virus-derived peptide
YEQHKITSY

SEQ ID NO: 37 - protein, 9 aa, Artificial Sequence
  virus-derived peptide
QEPMSIYVY

SEQ ID NO: 38 - protein, 10 aa, Artificial Sequence
  virus-derived peptide
SEHPTFTSQY

SEQ ID NO: 39 - protein, 9 aa, Artificial Sequence
  virus-derived peptide
QAIRETVEL

SEQ ID NO: 40 - protein, 9 aa, Artificial Sequence
  virus-derived peptide
TRATKMQVI

SEQ ID NO: 41 - protein, 8 aa, Artificial Sequence
  virus-derived peptide
DALPGPCI

SEQ ID NO: 42 - protein, 9 aa, Artificial Sequence
  virus-derived peptide
CEDVPSGKL

SEQ ID NO: 43 - protein, 9 aa, Artificial Sequence
  virus-derived peptide
HERNGFTVL

SEQ ID NO: 44 - protein, 13 aa, Artificial Sequence
  virus-derived peptide
PTFTSQYRIQGKL

SEQ ID NO: 45 - protein, 9 aa, Artificial Sequence
  virus-derived peptide
QMWQARLTV

SEQ ID NO: 46 - protein, 12 aa, Artificial Sequence
  virus-derived peptide
HELLVLVKKAQL

SEQ ID NO: 47 - protein, 12 aa, Artificial Sequence
  virus-derived peptide
DDYSNTHSTRYV

SEQ ID NO: 48 - protein, 9 aa, Artificial Sequence
  virus-derived peptide
SLYNTVATL

SEQ ID NO: 49 - protein, 9 aa, Artificial Sequence
  virus-derived peptide
GLCTLVAML

SEQ ID NO: 50 - protein, 9 aa, Artificial Sequence
  virus-derived peptide
GILGFVFTL

SEQ ID NO: 51 - protein, 9 aa, Artificial Sequence
  virus-derived peptide
STNRQSGRQ

SEQ ID NO: 52 - protein, 9 aa, Artificial Sequence
  virus-derived peptide
LLFGYPVYV

SEQ ID NO: 53 - protein, 9 aa, Artificial Sequence
  virus-derived peptide
FAEGFVRAL

SEQ ID NO: 54 - protein, 9 aa, Artificial Sequence
  virus-derived peptide
LIVIGILIL

SEQ ID NO: 55 - protein, 8 aa, Artificial Sequence
  virus-derived peptide
ILHTPGCV

SEQ ID NO: 56 - protein, 9 aa, Artificial Sequence
  virus-derived peptide
WYAQIQPHW

SEQ ID NO: 57 - protein, 9 aa, Artificial Sequence
  virus-derived peptide
AFSGVSWTM

SEQ ID NO: 58 - protein, 9 aa, Artificial Sequence
  virus-derived peptide
ILIGVVITW

SEQ ID NO: 59 - protein, 9 aa, Artificial Sequence
  virus-derived peptide
MMIPTVVAF

SEQ ID NO: 60 - protein, 9 aa, Artificial Sequence
  virus-derived peptide
PFPQSNAPI

SEQ ID NO: 61 - protein, 9 aa, Artificial Sequence
  virus-derived peptide
LLLTLLATV

SEQ ID NO: 62 - protein, 9 aa, Artificial Sequence
  virus-derived peptide
IVLEHGSCV

SEQ ID NO: 63 - protein, 9 aa, Artificial Sequence
  virus-derived peptide
LLFKTENGV

SEQ ID NO: 64 - protein, 9 aa, Artificial Sequence
  virus-derived peptide
PLNEAIMAV

SEQ ID NO: 65 - protein, 9 aa, Artificial Sequence
  virus-derived peptide
NLVRLQSGV

SEQ ID NO: 66 - protein, 9 aa, Artificial Sequence
  virus-derived peptide
LVISGLFPV

SEQ ID NO: 67 - protein, 9 aa, Artificial Sequence
  virus-derived peptide
LLLVAHYAI

SEQ ID NO: 68 - protein, 9 aa, Artificial Sequence
  virus-derived peptide
LALLAAFKV

SEQ ID NO: 69 - protein, 9 aa, Artificial Sequence
  virus-derived peptide
VILAGPMPV

SEQ ID NO: 70 - protein, 9 aa, Artificial Sequence
  virus-derived peptide
HVLGRLITV

SEQ ID NO: 71 - protein, 9 aa, Artificial Sequence
  virus-derived peptide
VTEHDTLLY

SEQ ID NO: 72 - protein, 9 aa, Artificial Sequence
  virus-derived peptide
NTDFRVLEL

SEQ ID NO: 73 - protein, 9 aa, Artificial Sequence
  virus-derived peptide
CVETMCNEY

SEQ ID NO: 74 - protein, 8 aa, Artificial Sequence
  virus-derived peptide
SSPPMFRV

SEQ ID NO: 75 - protein, 8 aa, Artificial Sequence
  control peptide
RALEYKNL

SEQ ID NO: 76 - protein, 888 aa, Artificial Sequence
  m38-pMHC-I-anti-FAP-IgG heavy chain fused to peptide-MHC-I
MGWSCIILFLVATATGVHSSSPPMFRVGCGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDAAGGPSVFIFPPKIKDVLMISLSPMVTCVVVDVSEDAPDVQISWFVNNVEVHTAQTQTHREDYNSTLRVVSALPIQHQDWMSGKEFKCKVNNRALGSPIEKTISKPRGPVRAPQVCVLPPPAEEMTKKEFSLSCAITGFLPAEIAVDWTSNGRTEQNYKNTATVLDSDGSYFMVSKLRVQKSTWERGSLFACSVVHEGLHNHLTTKTISRSLGK

SEQ ID NO: 77 - protein, 888 aa, Artificial Sequence
  IE3-pMHC-I-anti-FAP-IgG heavy chain fused to peptide-MHC-I
MGWSCIILFLVATATGVHSRALEYKNLGCGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDAAGGPSVFIFPPKIKDVLMISLSPMVTCVVVDVSEDAPDVQISWFVNNVEVHTAQTQTHREDYNSTLRVVSALPIQHQDWMSGKEFKCKVNNRALGSPIEKTISKPRGPVRAPQVCVLPPPAEEMTKKEFSLSCAITGFLPAEIAVDWTSNGRTEQNYKNTATVLDSDGSYFMVSKLRVQKSTWERGSLFACSVVHEGLHNHLTTKTISRSLGK

SEQ ID NO: 78 - protein, 470 aa, Artificial Sequence
  anti-FAP-IgG heavy chain
MGWSCIILFLVATATGVHSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDAAGGPSVFIFPPKIKDVLMISLSPMVTCVVVDVSEDAPDVQISWFVNNVEVHTAQTQTHREDYNSTLRVVSALPIQHQDWMSGKEFKCKVNNRALGSPIEKTISKPRGPVRAPQVYVLPPCAEEMTKKEFSLWCMITGFLPAEIAVDWTSNGRTEQNYKNTATVLDSDGSYFMYSKLRVQKSTWERGSLFACSVVHEGLHNRFTTKTISRSLGK

SEQ ID NO: 79 - protein, 234 aa, Artificial Sequence
  anti-FAP-IgG light chain
MGWSCIILFLVATATGVHSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 80 - protein, 135 aa, Artificial Sequence
  anti-FAP 28H1 VH
MGWSCIILFLVATATGVHSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 81 - protein, 127 aa, Artificial Sequence
  anti-FAP 28H1 VL
MGWSCIILFLVATATGVHSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 82 - protein, 15 aa, Artificial Sequence
  peptide linker
GCGGSGGGGSGGGGS

SEQ ID NO: 83 - protein, 20 aa, Artificial Sequence
  peptide linker
GGGGSGGGGSGGGGSGGGGS

SEQ ID NO: 84 - protein, 489 aa, Artificial Sequence
  anti-XCR1-IgG (MARX10) heavy chain with His Tag
MGWSCIILFLVATATGVHSQVQLQQPGAELVKPGASVKLSCKASGYTFTNYWIHWMKQRPGQGLEWIGMIHPNSDNTKYNEKFKAKAILTVDKSSSTAYMQLSSLTSEDSAVYYCARFANDGAYWGQGTLVTVSAAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDAAGGPSVFIFPPKIKDVLMISLSPMVTCVVVDVSEDAPDVQISWFVNNVEVHTAQTQTHREDYNSTLRVVSALPIQHQDWMSGKEFKCKVNNRALGSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGGGGGSLPETGGSGSHHHHHH

SEQ ID NO: 85 - protein, 238 aa, Artificial Sequence
  anti-XCR1-IgG (MARX10) light chain
MGWSCIILFLVATATGVHSDVVVTQTPLSLPVSLGDPASISCKSSQSLVHSNGNTYLHWYLQKPGQSPKLLIYKISNRFSGVPDRFSGSGSGTDFTLKISRVEAEDLGVYFCSQNTHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 86 - protein, 8 aa, Artificial Sequence
  peptide
SIINFEKL

(19) United States
(12) Patent Application Publication
(10) Pub. No.: US 2022/0073630 A1
(43) Pub. Date: Mar. 10, 2022

(54) A PEPTIDE-MHC-I-ANTIBODY FUSION PROTEIN FOR THERAPEUTIC USE IN A PATIENT WITH AMPLIFIED IMMUNE RESPONSE

(71) Applicants: HOFFMANN-LA ROCHE, INC., Little Falls, NJ (US); ROBERT KOCH-INSTITUT, Berlin (DE)

(72) Inventors: Hendrik KNOETGEN, Basael (CH); Cornelia FISCHER, Penzberg (DE); Richard KROCZEK, Berlin (DE)

(21) Appl. No.: 17/417,815

(22) PCT Filed: Dec. 18, 2019
(86) PCT No.: PCT/EP2019/086072

§ 371 Date: Jun. 24, 2021

(30) Foreign Application Priority Data
Dec. 28, 2018 (EP) 18248133.3
Mar. 7, 2019 (EP) 19161306.6

(51) Int. Cl. C07K 16/28; C07K 14/005; C07K 14/74; A61K 39/39; A61K 38/20; A61P 35/00

(52) U.S. Cl. CPC C07K 16/2866; C07K 14/005; C07K 14/70539; A61K 39/00; A61K 38/2013; A61P 35/00; C07K 2319/30; A61K 39/39

(57) ABSTRACT

The present invention relates to a peptide-MHC-I-antibody fusion protein for use as a therapeutic medicament administered to a patient, wherein a cellular cytotoxic immune response towards the virus-derived peptide has been amplified in the patient. The therapeutic medicament may be used in a kit of parts and administered in combination with an amplifying medicament and optionally an inducing medicament. The inducing medicament and the amplifying medicament may be for use in a method of making a patient susceptible for a treatment with the peptide-MHC-I-antibody fusion protein. The medicament(s) may be used for the treatment of diseases, such as cancer or a viral infection.

### FIELD OF THE INVENTION

The present invention relates to a peptide-MHC-I-antibody fusion protein for use as a therapeutic medicament administered to a patient, wherein a cellular cytotoxic immune response towards the virus-derived peptide has been amplified in the patient. The therapeutic medicament may be used in a kit of parts and administered in combination with an amplifying medicament and optionally an inducing medicament. The inducing medicament and the amplifying medicament may be for use in a method of making a patient susceptible for a treatment with the peptide-MHC-I-antibody fusion protein. The medicament(s) may be used for the treatment of diseases, such as cancer or a viral infection.

### BACKGROUND OF THE INVENTION

Peptide-MHC-I-antibody fusion proteins and their use for treatment of cancer and viral infections are known from WO 2012/175508, WO 2014/083004 and WO 2014/096015. Said peptide-MHC-I-antibody fusion proteins known in the art comprise a T cell response eliciting peptide (e.g. a virus-derived peptide) which is complexed with a class I MHC-I molecule and wherein said complex is coupled to a target cell binding antibody (see, e.g. the exemplary fusion protein in FIG. 1). Thereby, binding of the target cell binding antibody to a (diseased) target cell allows presentation of the peptide-loaded MHC-I complex on the surface of the target cell. This allows direction of peptide-specific T effector cells of the patient towards the target cells in order to mediate cell death. This mode-of-action requires presence of peptide-specific T effector cells. In patients with low levels of peptide-specific T cells a low response to a therapy with such peptide-MHC-I-antibody fusion proteins may be expected. There is a need to improve therapeutic application of peptide-MHC-I-antibody fusion proteins.

Chemokine receptor XCR1 is specifically expressed on dendritic cells (DC) involved in antigen cross-presentation to T effector cells (Kroczek et al. Front Immunol. 2012 Feb. 10; 3:14).

WO 2009/065561 discloses a system for delivering a substance into XCR1 positive cells. The delivery system includes an XCR1-binding component, e.g. an anti-XCR1 antibody or a fragment thereof. The substance may be a (poly)peptide. The substance is bound to the XCR1-binding component of the delivery system.

WO 2015/140172 and WO 2015/140175 disclose a method for extending a cellular cytotoxic immune response against an antigenic protein. The method includes administering to a patient peptide-loaded antigen presenting cells (APC); i.e. APC that present an antigenic peptide via their class I MHC I molecules in combination with an adjuvant. The administration is 0 hours to 14 days after the T cells of patient were activated against said antigen. The method may include further administration of one or more interleukins, including complexed IL-2 or a IL-2 mutein.

### SUMMARY OF THE INVENTION

The present invention relates to a peptide-MHC-I-antibody fusion protein, wherein the peptide-MHC-I-antibody fusion protein comprises
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule, for use as a therapeutic medicament,
   wherein the peptide-MHC-I-antibody fusion protein is to be administered to a patient, wherein a cellular cytotoxic immune response towards the virus-derived peptide has been induced in the patient and wherein the induced immune response towards the virus-derived peptide has been amplified in the patient by an amplifying medicament.

Therapeutic application of the peptide-MHC-I-antibody fusion protein mimics a viral infection in the target cells as the peptide-MHC-I complex comprised in said fusion protein is displayed on the surface of the target cell. By that, the natural, anti-viral CD8 T cell driven immune response is mediated. The involvement of a natural anti-viral T cell population may limit adverse effects in line with the better safety profile in terms of limited and selective release of cytokines in vitro (Schmittnaegel M, et al. Cancer Immunol Res. 2015; 3:764-76). With the medical use of the invention, the natural T cell response and thereby the potency of the treatment is improved by activation of peptide specific T cells prior to the actual treatment with the peptide-MHC-I-antibody fusion protein. Thereby a cellular cytotoxic immune response towards the virus-derived peptide is induced mediated by activation of the peptide-specific T cells. By amplifying peptide-specific T cells prior to the actual treatment with the peptide-MHC-I-antibody fusion protein response to the treatment may be triggered even in patients with initially low levels of peptide-specific T cells.

In one aspect a cellular cytotoxic immune response towards the virus-derived peptide is or has been induced by vaccination with an inducing medicament and/or by infection with a virus, thereby activating T cells against the virus-derived peptide. Suitable methods are disclosed in WO 2015/140172 and WO 2015/140175. Both documents are incorporated by reference herein. This immune response may be amplified in order to improve the ratio of virus-derived peptide specific CD8 T cells in a patient.

Therefore, in one aspect the invention relates to a peptide-MHC-I-antibody fusion protein, wherein the peptide-MHC-I-antibody fusion protein comprises
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule,
   for use as a therapeutic medicament, wherein the peptide-MHC-I-antibody fusion protein is to be administered to a patient, wherein a cellular cytotoxic immune response towards the virus-derived peptide has been induced in the patient and wherein the induced immune response towards the virus-derived peptide has been amplified in the patient by an amplifying medicament,
   wherein the cellular cytotoxic immune response towards the virus-derived peptide has been induced by vaccination with an inducing medicament and/or by infection with a virus, thereby activating T cells against the virus-derived peptide; and/or wherein the amplifying medicament comprises a class I MHC expressing, antigen presenting cell, wherein the MHC-I is bound to the virus-derived peptide, and a Th1 adjuvant, which is a danger signal.

In one aspect the invention relates to a peptide-MHC-I-antibody fusion protein, wherein the peptide-MHC-I-antibody fusion protein comprises
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule,
   for use as a therapeutic medicament,
   wherein the peptide-MHC-I-antibody fusion protein is to be administered to a patient, wherein a cellular cytotoxic immune response towards the virus-derived peptide has been induced in the patient by an inducing medicament and wherein the induced immune response towards the virus-derived peptide has been amplified in the patient by an amplifying medicament. wherein the inducing medicament comprises
(a) an antibody or a variant or fragment thereof, specifically binding to chemokine (C motif) receptor 1 (XCR1), and
(b) the virus-derived peptide, wherein the peptide is covalently coupled to said antibody or the variant or fragment thereof, and wherein the amplifying medicament comprises a class I MHC expressing, antigen presenting cell, wherein the MHC-I is bound to the virus-derived peptide, and a Th1 adjuvant, which is a danger signal.

In one aspect the invention relates to a peptide-MHC-I-antibody fusion protein, wherein the peptide-MHC-I-antibody fusion protein comprises
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule,
   for use as a therapeutic medicament,
   wherein the peptide-MHC-I-antibody fusion protein is to be administered to a patient, wherein a cellular cytotoxic immune response towards the virus-derived peptide has been induced in the patient by an inducing medicament and wherein the induced immune response towards the virus-derived peptide has been amplified in the patient by an amplifying medicament. wherein the inducing medicament comprises
(a) lymphotactin (XCL1) or a variant or fragment thereof, specifically binding to XCR1, and
(b) the virus-derived peptide, wherein the peptide is covalently coupled to XCL1 or a variant or fragment thereof, and wherein the amplifying medicament comprises a class I MHC expressing, antigen presenting cell, wherein the MHC-I is bound to the virus-derived peptide, and a Th1 adjuvant, which is a danger signal.

Another aspect of the invention is a kit of parts comprising an amplifying medicament and a therapeutic medicament,
the amplifying medicament comprising
(a) a class I MHC expressing, antigen presenting cell, wherein the MHC-I is bound to the virus-derived peptide, and
(b) a Th1 adjuvant, which is a danger signal, wherein the amplifying medicament amplifies an immune response towards a virus-derived peptide in a patient, and
the therapeutic medicament comprising
(i) an antibody specifically binding to a target cell, and
(ii) a polypeptide comprising in N- to C-terminal direction the virus-derived peptide, a β2-microglobulin and the extracellular domains α1, α2, and α3 of a class I MHC molecule.

The kit may further comprise an inducing medicament inducing a cellular cytotoxic immune response towards the virus-derived peptide in the patient.

The kit may be used as a medicament, wherein the therapeutic medicament is to be administered to a patient with amplified immune response towards the virus-derived peptide and wherein the inducing medicament induces the cellular cytotoxic immune response towards the virus-derived peptide in the patient by vaccination, particularly wherein the inducing medicament is as defined above.

Another aspect of the invention is an antibody-peptide fusion protein comprising
(a) an antibody specifically binding to chemokine (C motif) receptor 1 (XCR1), and
(b) a virus-derived peptide, wherein the peptide is covalently coupled to said antibody,
   for use as an inducing medicament, wherein the antibody-peptide fusion protein is to be administered to a patient in a vaccination in combination with
a class I MHC expressing, antigen presenting cell, wherein the MHC-I is bound to the virus-derived peptide, and an adjuvant which supports a Th1 mediated response,
   wherein the vaccination is the first step of a combination therapy, wherein the vaccination is to be administered prior to the second step of a combination therapy, in which a therapeutic medicament comprising a peptide-MHC-I-antibody fusion protein is to be administered to the patient, wherein the peptide-MHC-I-antibody fusion protein comprises
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule.

Another aspect of the invention is a kit of parts comprising an inducing medicament and an amplifying medicament,
the inducing medicament being defined as above, and
the amplifying medicament being defined as above, for use in a method of making a patient susceptible for a treatment with the peptide-MHC-I-antibody fusion protein comprising
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule.

In one embodiment of the invention the inducing medicament has been or is administered in combination with a Th1 adjuvant, which is a danger signal.

In another embodiment of the invention interleukin-2 (IL-2) or a IL-2 variant with extended half-life in the patient has been or is administered after the administration of the class I MHC expressing, antigen presenting cell.

In still another embodiment of the invention the Th1 adjuvant is polyinosinic:polycytidylic acid (poly I:C).

Yet, in another embodiment of the invention the class I MHC expressing, antigen presenting cell, and the adjuvant have been or are administered 4 days to 11 days after the T cells of patient were activated against said virus-derived peptide.

In another embodiment of the invention the therapeutic medicament is administered when the level of virus-derived peptide-specific CD8+ T cells is elevated as a result of the administration of the amplifying medicament, preferably wherein the therapeutic medicament is administered within 29 days, particularly 20 days, especially 14 days, from the administration of the amplifying medicament. More preferably, the therapeutic medicament is administered shortly after the amplification, such as within 3, 2 or 1 day from the administration of the amplifying medicament or immediately thereafter.

In still another embodiment of the invention the antibody comprised in the peptide-MHC-I-antibody fusion protein specifically binds to a target cell selected from the group of cancer cells and virus-infected cells.

In still another embodiment of the invention, the peptide-MHC-I-antibody fusion protein for use, or the kit of parts is for use in treating cancer or a viral infection.

Another aspect of the invention is a method for treating a cancer, wherein the method comprises the steps of
amplifying the cellular cytotoxic immune response towards a virus-derived peptide with an amplifying medicament comprises a class I MHC expressing, antigen presenting cell, wherein the MHC-I is bound to the virus-derived peptide, and a Th1 adjuvant, which is a danger signal, and
administering an effective amount of a peptide-MHC-I-antibody fusion protein, wherein the peptide-MHC-I-antibody fusion protein comprises
   (i) an antibody specifically binding to a target cell, and
   (ii) a polypeptide comprising in N- to C-terminal direction the virus-derived peptide, a β2-microglobulin and the extracellular domains α1, α2, and α3 of a class I MHC molecule

to a patient in need thereof.

Another aspect of the invention is a method for treating a viral infection, wherein the method comprises the steps of
inducing a cellular cytotoxic immune response towards a virus-derived peptide with an inducing medicament,
amplifying the cellular cytotoxic immune response towards a virus-derived peptide with an amplifying medicament comprises a class I MHC expressing, antigen presenting cell, wherein the MHC-I is bound to the virus-derived peptide, and a Th1 adjuvant, which is a danger signal, and
administering an effective amount of a peptide-MHC-I-antibody fusion protein, wherein the peptide-MHC-I-antibody fusion protein comprises
(i) an antibody specifically binding to a target cell, and
(ii) a polypeptide comprising in N- to C-terminal direction the virus-derived peptide, a β2-microglobulin and the extracellular domains α1, α2, and α3 of a class I MHC molecule
to a patient in need thereof.

Another aspect of the invention is the use of a peptide-MHC-I-antibody fusion protein, wherein the peptide-MHC-I-antibody fusion protein comprises
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule,
   for the manufacture of a medicament for treating cancer or a viral infection, wherein the peptide-MHC-I-antibody fusion protein is to be administered to a patient, wherein a cellular cytotoxic immune response towards the virus-derived peptide has been induced in the patient and wherein the induced immune response towards the virus-derived peptide has been amplified in the patient by an amplifying medicament.

According to the invention an induced immune response towards the virus-derived peptide is/has been amplified in a patient by an amplifying medicament. The amplifying medicament preferably comprises a class I MHC expressing, antigen presenting cell, wherein the MHC-I is bound to the virus-derived peptide, and a Th1 adjuvant, which is a danger signal. The amplification effects that the ratio of virus-derived peptide-specific CD8 T cells in a patient is improved. Subsequent disease therapy with peptide-MHC-I-antibody fusion proteins, e.g. as disclosed in WO 2012/175508, WO 2014/083004 and WO 2014/096015, may be improved, e.g. by inhibition of metastasis formation. Patients with low initial levels of virus-derived peptide-specific T cells may respond better to a therapy with such peptide-MHC-I-antibody fusion proteins.

### DESCRIPTION OF THE FIGURES

FIG. 1: Schematic illustration of an exemplary peptide-MHC-I-antibody fusion protein as used in the Examples.

FIG. 2: Frequency of IFNγ expressing CD8+ T cells measured in FACS according to Example 3 after exposure to pMHC-I-IgG-treated target cells.

FIG. 3: Specific tumor cell lysis mediated by pMHC-I-IgGs after incubation with freshly isolated splenocytes from m38-vaccinated mice as assessed in Example 4.

FIG. 4: Assessment of metastasis burden after preventive treatment according to Example 5.

FIG. 5: Assessment of metastasis burden after therapeutic treatment of experimental lung metastases according to Example 6.

### DETAILED DESCRIPTION OF THE INVENTION

### (a) Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

Unless otherwise defined herein the term “comprising of” shall include the term “consisting of”.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The “light chains” of antibodies (immunoglobulins) from any vertebrate species can be assigned to one of two distinct types, called kappa (κ) and lambda (λ), based on the amino acid sequences of their constant domains. A wild type light chain typically contains two immunoglobulin domains, usually one variable domain (VL) that is important for binding to an antigen and a constant domain (CL).

Several different types of “heavy chains” exist that define the class or isotype of an antibody. A wild type heavy chain contains a series of immunoglobulin domains, usually with one variable domain (VH) that is important for binding antigen and several constant domains (CH1, CH2, CH3, etc.).

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The “class” of an antibody refers to the type of constant domain or constant region possessed by its heavy chain. Depending on the amino acid sequence of the constant region of their heavy chains, antibodies are divided in the classes: IgA, IgD, IgE, IgG and IgM, and several of these may are further divided into subclasses (isotypes), such as IgG1, IgG2, IgG3, and IgG4, IgA1 and IgA2. The heavy chain constant regions that correspond to the different classes of antibodies are called α, δ, ε, γ and μ, respectively, respectively. The light chain constant regions (CL) which can be found in all five antibody classes are called κ (kappa) and λ (lambda). The “constant domains” as used herein are from human origin, which is from a constant heavy chain region of a human antibody of the subclass (isotype) IgG1, IgG2, IgG3, or IgG4 and/or a constant light chain kappa or lambda region. Such constant domains and regions are well known in the state of the art and e.g. described by Kabat, et al., Sequences of Proteins of Immunological Interest, 5th ed., Public Health Service, National Institutes of Health, Bethesda, Md. (1991).

The term “Fc domain” or “Fc region” is used herein to define a C-terminal region of an immunoglobulin heavy chain that contains at least a portion of the constant region. For example in natural antibodies, the Fc domain is composed of two identical protein fragments, derived from the second and third constant domains of the antibody's two heavy chains in IgG, IgA and IgD isotypes; IgM and IgE Fc domains contain three heavy chain constant domains (CH domains 2-4) in each polypeptide chain. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The terms “full length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. In certain embodiments, a human antibody is derived from a non-human transgenic mammal, for example a mouse, a rat, or a rabbit. In certain embodiments, a human antibody is derived from a hybridoma cell line.

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The terms “binding site” or “antigen-binding site” as used herein denotes the region(s) of an antibody molecule to which a ligand (e.g. the antigen or antigen fragment of it) actually binds and which is derived from an antibody. The antigen-binding site includes antibody heavy chain variable domains (VH) and/or antibody light chain variable domains (VL), or pairs of VH/VL.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The terms “monoclonal antibody” or “monoclonal antibody composition” as used herein refer to a preparation of antibody molecules of a single amino acid composition.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “constant domains” or “constant region” as used within the current application denotes the sum of the domains of an antibody other than the variable region. The constant region is not directly involved in binding of an antigen, but exhibits various effector functions.

An epitope is a region of an antigen that is bound by an antibody. The term “epitope” includes any polypeptide determinant capable of specific binding to an antibody. In certain embodiments, epitope determinants include chemically active surface groupings of molecules such as amino acids, glycan side chains, phosphoryl, or sulfonyl, and, in certain embodiments, may have specific three dimensional structural characteristics, and/or specific charge characteristics. In certain embodiments, an antibody is said to specifically bind an antigen when it preferentially recognizes its target antigen in a complex mixture of proteins and/or macromolecules.

As used herein, the terms “binding” and “specific binding” refer to the binding of the antibody to an epitope of the antigen in an in vitro assay, preferably in a plasmon resonance assay (BIAcore, GE-Healthcare Uppsala, Sweden) with purified wild-type antigen.

The affinity of the binding of an antibody to an antigen is defined by the terms ka (rate constant for the association of the antibody from the antibody/antigen complex), kD (dissociation constant), and KD (kD/ka). In one embodiment binding or that/which specifically binds to means a binding affinity (KD) of 10⁻⁸ mol/1 or less, in one embodiment 10⁻⁸ M to 10⁻¹³ mol/l. Thus, an antibody specifically binds to each antigen for which it is specific with a binding affinity (KD) of 10⁻⁸ mol/1 or less, e.g. with a binding affinity (KD) of 10⁻⁸ to 10⁻¹³ mol/l. in one embodiment with a binding affinity (KD) of 10⁻⁹ to 10⁻¹³ mol/l.

The term “amino acid” as used herein denotes an organic molecule possessing an amino moiety located at α-position to a carboxylic group. Examples of amino acids include: arginine, glycine, ornithine, lysine, histidine, glutamic acid, asparagic acid, isoleucine, leucine, alanine, phenylalanine, tyrosine, tryptophan, methionine, serine, proline. The amino acid employed is optionally in each case the L-form. The term “positively charged” or “negatively charged” amino acid refers to the amino acid side-chain charge at pH 7.4. Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

TABLE
Amino acids with specific properties

| | 3- | 1- | Side-chain | Side-chain charge |
|---|---|---|---|---|
| Amino Acid | Letter | Letter | polarity | (pH 7.4) |
| Alanine | Ala | A | nonpolar | neutral |
| Arginine | Arg | R | basic polar | positive |
| Asparagine | Asn | N | polar | neutral |
| Aspartic acid | Asp | D | acidic polar | negative |
| Cysteine | Cys | C | nonpolar | neutral |
| Glutamic acid | Glu | E | acidic polar | negative |
| Glutamine | Gln | Q | polar | neutral |
| Glycine | Gly | G | nonpolar | neutral |
| Histidine | His | H | basic polar | positive (10%) |
| | | | | neutral (90%) |
| Isoleucine | Ile | I | nonpolar | neutral |
| Leucine | Leu | L | nonpolar | neutral |
| Lysine | Lys | K | basic polar | positive |
| Methionine | Met | M | nonpolar | neutral |
| Phenylalanine | Phe | F | nonpolar | neutral |
| Proline | Pro | P | nonpolar | neutral |
| Serine | Ser | S | polar | neutral |
| Threonine | Thr | T | polar | neutral |
| Tryptophan | Trp | W | nonpolar | neutral |
| Tyrosine | Tyr | Y | polar | neutral |
| Valine | Val | V | nonpolar | neutral |

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991), which is referred to herein as “numbering according to Kabat et al.”. For the constant heavy chain domains (CH1, Hinge, CH2 and CH3) the Kabat EU index numbering system (see pages 661-723) is used and is herein referred to as “numbering according to EU index of Kabat”.

Amino acid substitutions (or mutations) within the polypeptide chains of the multispecific antibody are prepared by introducing appropriate nucleotide changes into the antibody DNA, or by nucleotide synthesis. Such modifications can be performed, however, only in a very limited range, e.g. as described above. For example, the modifications do not alter the above mentioned antibody characteristics such as the IgG isotype and antigen binding, but may further improve the yield of the recombinant production, protein stability or facilitate the purification. In certain embodiments, antibody variants having one or more conservative amino acid substitutions are provided.

The term “tertiary structure” as used herein refers to the geometric shape of the antibody according to the invention. The tertiary structure comprises a polypeptide chain backbone comprising the antibody domains, while amino acid side chains interact and bond in a number of ways.

A “polypeptide” is a polymer consisting of amino acids joined by peptide bonds, whether produced naturally or synthetically. Polypeptides of less than about 25 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups, metal ions, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

The term “virus-derived peptide” as used herein refers to a peptide derived from a virus. The term “derived from” in this context means that the peptide is identical to a naturally occurring T cell epitope of the virus (i.e. shares the same amino acid sequence). In other words, the amino acid sequence of the virus-derived peptide is identical to a partial sequence of the amino acid sequence of a protein antigen of the virus. Although the virus-derived peptide used for the invention shares the same amino acid sequence as the corresponding naturally occurring T cell epitope, virus-derived peptides comprised in fusion proteins for use according to the invention are typically of synthetic origin.

Human cytomegalovirus HCMV (=Human herpesvirus 5, HHV-5) is one of the largest human viruses. Its genome comprises around 230,000 bp linear double stranded DNA and encodes more than 160 proteins (Davison, A. J., et al., J. Gen. Virol. 84 (2003) 17-28).

By “covalent coupling” or “covalent binding” of the virus-derived peptide to a protein, e.g. an antibody, is meant a coupling by a chemical bond involving the sharing of electron pairs between an atom of the virus-derived peptide and an atom of said protein.

A “peptide” as used herein is a short chain of amino acid monomers linked by peptide bonds. Peptides that bind to MHC class I are typically 8 to 12 amino acids in length. Hence, a “virus-derived peptide” as used herein typically comprises 8 to 12 amino acids.

An “epitope” is the part of a protein antigen that is recognized by the immune system, e.g. by antibodies, B cells or T cells. A “T cell epitope” is presented on the surface of a cell, where it is bound to MHC molecules. T cell epitopes presentable by MHC I can be bound by the T cell receptor of cytotoxic CD8 T lymphocytes (CD8 T cells or CTLs). T cell epitopes presentable by MHC I are typically peptides of 8 to 12 amino acids in length. A class I MHC molecule and a peptide bound thereto is herein also referred to as “peptide-MHC-I complex” or simply “complex”.

Class I MHC molecules are heterodimers that consist of two polypeptide chains, α (comprised of domains α1, α2, and α3) and β2-microglobulin (b2m). The two chains are linked noncovalently via interaction of b2m and the 3 domain.

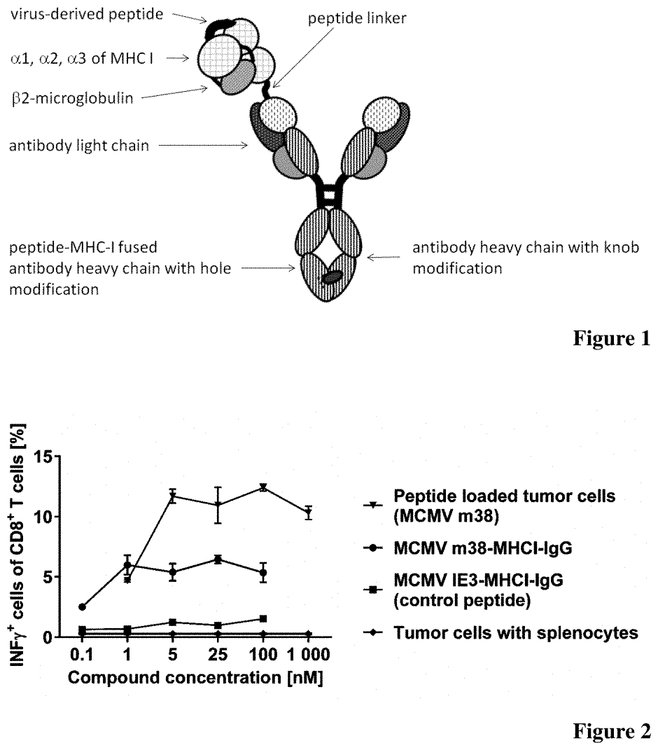

A “peptide-MHC-I-antibody fusion protein” as used herein is a recombinant fusion protein comprising a peptide-loaded MHC-I complex coupled to an antibody, e.g. as disclosed in WO 2012/175508, WO 2014/083004 and WO 2014/096015 and as illustrated in an exemplary schematic in FIG. 1. Peptide-MHC-I-antibody fusion protein are produced by recombinant methods and means known in the art, e.g. from WO 2012/175508, WO 2014/083004 and WO 2014/096015. A “peptide-MHC-I-antibody fusion protein” comprises a “polypeptide” comprising in N- to C-terminal direction a virus-derived peptide, a β2-microglobulin and the extracellular domains α1, α2, and α3 of a class I MHC molecule. Hence, all components of the class I MHC molecule as well as the virus-derived peptide are provided on a single polypeptide chain so that the polypeptide comprised in the peptide-MHC-I-antibody fusion protein is also referred to as “single chain peptide-MHC-I-complex”.

“Fused” and “connected” with respect to polypeptides refers to components that are linked by peptide bonds, either directly or via one or more peptide linkers. The term “peptide linker” as used herein denotes a peptide of an amino acid sequence, which is preferably of synthetic origin. Typically the peptide connectors are composed of flexible residues like glycine and serine so that the adjacent protein domains are free to move relative to one another. Thus, typical peptide connectors used in accordance with the invention are glycine-serine linkers, i.e. peptide connectors consisting of a pattern of glycine and serine residues. The amino acid sequence of a peptide linker used herein typically has a length of 8 to 50 amino acid residues. Typical peptide linkers are glycin-serine linkers.

The single chain peptide-MHC-I-complex comprised in a peptide-MHC-I-antibody fusion protein may comprise a disulfide bridge. In this case, a disulfide bond is formed between a cysteine residue of the first peptide linker, i.e. the peptide linker connecting the virus-derived peptide with the β2-microglobulin, and a cysteine residue of the extracellular domains α1, α2, and α3 of the class I MHC molecule.

“Inducing a cellular cytotoxic immune response towards the virus-derived peptide” as used herein refers to a naturally occurring viral infection of a patient or a treatment administered to a patient that leads to activation of virus-derived-peptide-specific CD8 T cells, thereby leading to induction of cellular immune responses towards virus-derived peptide loaded cells in the patient. The administered treatment is referred to as “inducing medicament”. Suitable methods are, e.g. disclosed in WO 2015/140172 and WO 2015/140175. One exemplary “inducing medicament” is a molecule that binds to XCR1, e.g. an antibody or lymphotactin (or fragments or variants thereof), wherein the molecule is coupled to a virus-derived peptide. The cellular cytotoxic immune response towards the virus-derived peptide may also be induced by a natural viral infection. For example, around 50% of the population in Europe and the US is chronically infected by hCMV. With patients which already exhibit CD8 T cell immune response (e.g. more than 0.005% of all CD8 T cells measurable by the tetramer-technology), no active immunization/vaccination (treatment with an inducing medicament as defined herein) is not required. The levels of CD8⁺ cytotoxic T cells specific for hCMV tend to rise towards older age and can reach in some cases several percent. Usually the levels are around 1% and can be clearly lower. The other about 50% of the population are not infected by hCMV and therefore do not have any pre-existing CD8⁺ T cell cytotoxic activity against proteins of this virus. An efficient vaccination in the human system (e.g. by in vivo targeting antigen into XCR1⁺ DC) can be expected to result in around 0.03 to 0.1% antigen-specific CD8⁺ T cells (relative to all CD8 T cells).

“Amplifying the cellular cytotoxic immune response towards the virus-derived peptide” as used herein refers to a treatment administered to a patient that leads to amplification of the number of virus-derived-peptide-specific CD8 T cell in the patient. The administered treatment is referred to as “amplifying medicament”. One exemplary “amplifying medicament” comprises a class I MHC expressing, antigen presenting cell, wherein the MHC-I is bound to the virus-derived peptide, and a Th-1 adjuvant, which is a danger signal. The induced cellular cytotoxic immune response towards the virus-derived peptide may not be sufficient to effectively eradicate cancer tissue using the therapeutic medicament. The levels of virus-derived peptide-specific CD8⁺ T cell cytotoxic activity after induction as described above are usually too low for allowing efficient treatment with the therapeutic medicament according to the invention. For example, the low levels of hCMV-specific CD8⁺ T cell cytotoxic activity in these chronically infected persons are most likely not sufficient to effectively eradicate cancer tissue using the therapeutic medicament. The same holds true for patient having been vaccinated with an inducing medicament. Repeating vaccination does usually not significantly raise the percentage of antigen-specific CD8⁺ T cells. This means that active immunization does usually not achieve higher levels of antigen-specific CD8⁺ T cell-based cytotoxicity compared to chronic infection with hCMV. Following vaccination or natural infection, the immune response can be highly amplified e.g. by administering (i) class I MHC expressing, antigen presenting cells, wherein the MHC-I is bound to the virus-derived peptide, and (ii) a Th1 adjuvant, which is a danger signal. The class I MHC expressing, antigen presenting cells may be e.g. autologous PBMCs. Alternatively to PBMC, one could use also in vitro expanded T cells or B cells. The amplification (re-activation) of the induced virus-derived peptide-specific (e.g. hCMV-specific) CD8⁺ T cells by peptide-loaded autologous cells expands the antigen-specific CD8 T cells and also makes them more cytotoxic. Additional administration of an appropriate form of IL-2 strongly further expands the antigen-specific CD8 T cells. At the same time, these T cells become more cytotoxic (through the action of IL-2). As detailed above, with patients which already exhibit CD8 T cell immune response no active immunization/vaccination (i.e. treatment with an inducing medicament as defined herein) is necessary. In fact, active immunization by targeting an antigen into XCR1⁺ dendritic cells would not significantly raise these levels of antigen-specific CD8⁺ T cells in these patients. Thus, the treatment in these patients can be very significantly simplified and made more cost-effective by amplifying the immune response as described above. Low levels of antigen-specific (active or memory) CD8⁺ T cells can very effectively be amplified by administering the amplifying medicament. Amplifying medicaments comprising class I MHC expressing, antigen presenting cells, wherein the MHC-I is bound to a peptide, and a Th-1 adjuvant, which is a danger signal, and further details on the amplification of the cellular cytotoxic immune response towards a peptide are described in application EP 18213650.

An “peptide-comprising fusion protein” as used herein is a recombinant fusion protein comprising lymphotactin (XCL1), or a variant or fragment thereof, and a virus-derived peptide. XCL1, which is also known as ATAC, lymphotactin or SCM-1 is the only member of the C family of chemokines. Activation-induced, T cell-derived, and chemokine-related cytokine (ATAC) was cloned in the human (Müller et al., 1995, Eur. J. Immunol. 25, 1744-48), and independently as lymphotactin (Kelner et al., 1994, Science 266, 1395-99) in the mouse and SCM-1 (Yoshida et al., 1995, FEBS Lett. 360, 155-9) in human. According to the nomenclature on chemokines ATAC/lymphotactin/SCM-1 is now designated “XCL1”. XCL1 is secreted mainly by activated CD8⁺ T-cells, Th1 CD4⁺ T cells and by NK cells. In the human, a variant of XCL1 designated XCL2 has been described in which the amino acids aspartate and lysine in position 28 and 29 of the full length protein are exchanged for histidine and arginine, respectively (Yoshida et al., 1996, FEBS Lett. 395, 82-8), which may also be used for the present invention. An exemplary method to produce XCL1 in biologically active form is described in Example 8 of WO 2009/065561. Analogous methods may be used in order to produce other biologically active forms of XCL1, e.g. those of other species. XCL1 is a ligand of XCR1.

The lymphotactin (XCL1) or variant thereof or fragment thereof is capable of specifically binding to XCR1 in order to induce the immune response towards the virus-derived peptide, which is bound thereto. The amino acid sequences of XCL1 of several species (including human: GenBank accession P47992; mouse: GenBank accession P47993; and rat: GenBank accession P51672) are known. Additionally, a specific XCLR1 agonist referred to as K4.1 HHV8 (GenBank accession AAB62672.1), which is a viral chemokine-like protein, is also known. Any of these naturally occurring XCR1 ligands or any other natural occurring XCR1 ligand may be used.

A “peptide-comprising fusion protein” as used herein may comprise a functionally active variant of any naturally occurring XCL1. The term “variant” encompasses fragments, variants derived by one or more amino acid additions, deletions and/or substitutions and molecules, particularly proteins, comprising any naturally occurring XCL1 or part thereof, such as fusion proteins. The XCL1 portion of the fusion protein may be flanked by the amino acid residue(s) C-terminally, N-terminally, or C- and N-terminally, especially C-terminally only. The functionally active fragment is characterized by being derived from any natural occurring XCR1 ligand, particularly XCL1, by one or more amino acid deletions. The deletion(s) may be C-terminally, N-terminally and/or internally. Preferably, the fragment is obtained by at most 1, 2, 3, 4, 5, 6, 7, 8, 9, 10, 20, 30, 40, 50 or 60, more preferably by at most 1, 2, 3, 4, 5, 6, 7, 8, 9, 10, 15, 20, 25 or 30, even more preferably at most 1, 2, 3, 4, 5, 6, 7, 8, 9, 10, 11, 12, 13, 14 or 15, still more preferably at most 1, 2, 3, 4, 5, 6, 7, 8, 9 or 10, most preferably 1, 2, 3, 4 or 5 amino acid deletion(s). The functionally active fragment of the invention is characterized by having a biological activity similar to that displayed by the ligand from which it is derived, including the ability to binding to XCR1 and mediate internalization of the virus-derived peptide. The fragment of the naturally occurring XCR1 ligand, particularly XCL1, is functionally active in the context of the present invention, if the activity (binding as well as internalization) of the fragment amounts to at least 10%, preferably at least 25%, more preferably at least 50%, even more preferably at least 70%, still more preferably at least 80%, especially at least 90%, particularly at least 95%, most preferably at least 99% of the activity of the XCL1 without sequence alteration. These fragments may be designed or obtained in any desired length, including as small as about 18 to 50 amino acids in length. The functionally active fragment of the naturally occurring XCR1 ligand, particularly XCL1, may be also characterized by other structural features. Accordingly, in one preferred embodiment of the invention the functionally active fragments consists of at least 60%, preferably at least 70%, more preferably at least 80%, still more preferably at least 90%, even more preferably at least 95%, most preferably 99% of the amino acids of the XCR1 ligand. The functional active fragment as defined above may be derived from the peptide by one or more amino acid deletions. The deletions are preferably C-terminally and/or internally. Another preferred embodiment of the invention relates to a XCL1 variant, wherein the XCR1 ligand is a functionally active variant of an XCR1 ligand and wherein the variant has at least 50% sequence identity to the XCR1 ligand. In a more preferred embodiment the functionally active variant has a sequence identity of at least 60%, preferably at least 70%, more preferably at least 80%, still more preferably at least 90%, even more preferably at least 95%, most preferably 99% to XCL1. The functional active variant is obtained by sequence alterations in the naturally occurring XCR1 ligand, wherein the XCR1 ligand with the sequence alterations retains a function of the unaltered XCR1 ligand, e.g. having a biological activity similar to that displayed by the naturally occurring XCR1 ligand, including the ability to binding to XCR1 and mediate internalization of a substance ii). Such sequence alterations can include, but are not limited to, conservative substitutions, deletions, mutations and insertions. These characteristics of the functional active variant can be assessed e.g. as detailed above. In a still more preferred embodiment of the invention the functionally active variant of an is derived from the naturally occurring XCR1 ligand by conservative substitutions. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc. In one embodiment, one conservative substitution is included in the peptide. In another embodiment, two conservative substitutions or less are included in the peptide. In a further embodiment, three conservative substitutions or less are included in the peptide.

An “antibody-peptide fusion protein” as used herein is a recombinant fusion protein comprising an antibody or variant thereof or fragment thereof specifically binding to chemokine (C motif) receptor 1 (XCR1) and a virus-derived peptide. The mode of action of the antibody-peptide fusion protein is to deliver the virus-derived peptide to professional antigen presenting cells, i.e. XCR1-expressing dendritic cells according to the general concept as disclosed in WO 2009/065561 A2.

Chemokine (C motif) receptor 1 (“XCR1”) is present on the surface of professional antigen-presenting cells, particularly dendritic cells (DC), and can be used to selectively deliver substance into these cells. Targeted delivery of a substance to XCR1-expressing DC allows induction of a potent Th1 immune reaction in mammals/humans, when applied together with a Th1 adjuvant which is a danger signal. The term “XCR1” as used herein, refers to any native XCR1 from any vertebrate source, including mammals such as primates (e.g. humans) and rodents (e.g., mice and rats), unless otherwise indicated. The term encompasses “full-length,” unprocessed XCR1 as well as any form of XCR1 that results from processing in the cell. The term also encompasses naturally occurring variants of XCR1, e.g., splice variants or allelic variants. The amino acid sequence of an exemplary human XCR1 is shown in SEQ ID NO:1 (derived from NCBI; accession NP_001019815).

Antibodies specifically binding to XCR1 are, e.g. disclosed in EP 2 641 915 A1. Antibodies specifically binding to XCR1 may be provided by general methods and means known in the art, e.g. by screening combinatorial libraries for antibodies with the desired activity or activities or by immunization. Antibodies specifically binding to XCR1 are herein also referred to as “anti-XCR1 antibodies”.

The anti-XCR1 antibody or variant thereof or fragment thereof is capable of specifically binding to XCR1 in order to induce the immune response towards the virus-derived peptide, which is bound thereto. In one embodiment of the invention an antibody or a variant or fragment thereof, specifically binding to XCR1, may be used along with a virus-derived peptide, wherein the peptide is covalently coupled to said antibody or a variant or fragment thereof in order to induce the immune response towards the virus-derived peptide. The variant or fragment is functionally active, i.e. the functionally active variant/fragment (a) is characterized by being derived from any anti-XCR1 antibody by one or more amino acid deletions, additions and/or substitutions, such as C-terminal, N-terminal and/or internal deletions, additions and/or substitutions, and (b) is characterized by having a biological activity similar to that displayed by the anti-XCR1 antibody from which it is derived, including the ability to bind to XCR1. Antibodies generated against XCR1 can be obtained by direct injection of XCR1 or a fragment thereof into an animal or by administering XCR1 or a fragment thereof to an animal, preferably a non-human. The antibody so obtained will then bind to XCR1. For preparation of monoclonal antibodies, any technique known in the art, which provides antibodies produced by continuous cell line cultures, e.g. a hybridoma cell line, can be used. The variant is essentially defined analogous to the lymphotactin variant.

Anti-XCR1 antibodies may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, N.J., 2001) and in Marks and Bradbury in Methods in Molecular Biology 248:161-175 (Lo, ed., Human Press, Totowa, N.J., 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. in EMBO Journal 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in the Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antibodies or antibody fragments isolated from human antibody libraries are considered human antibodies or human antibody fragments herein.

The fusion proteins for use according to the invention are produced by recombinant means. Methods for recombinant production of proteins are widely known in the state of the art and comprise protein expression in prokaryotic and eukaryotic cells with subsequent isolation of the antibody and usually purification to a pharmaceutically acceptable purity. For the expression of the fusion proteins as aforementioned in a host cell, nucleic acids encoding the respective polypeptide chains are inserted into expression vectors by standard methods. Expression is performed in appropriate prokaryotic or eukaryotic host cells, like CHO cells, NS0 cells, SP2/0 cells, HEK293 cells, COS cells, PER.C6 cells, yeast, or E. coli cells, and the antibody is recovered from the cells (supernatant or cells after lysis). General methods for recombinant production of proteins are well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

Adjuvants in the field of pharmacology are drugs that have few or no pharmacological effects by themselves, but may increase the efficacy or potency of other drugs when given at the same time. In immunology, an adjuvant is an agent which, while not having any specific antigenic effect in itself, may stimulate the immune system, increasing the response to a vaccine. In some documents of the prior art, cytokines, such IL-2, are wrongly designated as being “adjuvants” in the context of T cell responses. Such wrong designation is both attributable to the incorrect use of the term “adjuvant”, which is generally accepted to refer to an agent which modifies the effect of other agents while having few if any direct effects when given by itself. Cytokines, such as IL-2, however, are known to have numerous effects on their own, and do not represent an “adjuvant” according to the present invention. In any event, cytokines, such as IL-2 are not “Th1 adjuvants which are a danger signal” according to the present invention. Accordingly, in another preferred embodiment of the present invention, the Th1 adjuvant which is a danger signal is not a cytokine. In another more preferred embodiment of the present invention, the Th1 adjuvant which is a danger signal is not IL-2.

A “Th1 adjuvant which is a danger signal” is understood to be selected from a Pathogen-Associated Molecular Pattern (PAMP, e.g. LPS), a derivative of a PAMP (e.g. MPL and GLA as derivatives of LPS), or an artificial compound, such as a peptide, protein, lipid, lipoprotein, carbohydrate, nucleic acid or small molecule, mimicking a PAMP and activating one or several Pattern Recognition Receptors (PRR). Examples of such artificial compounds are poly J:Q, mimicking double-stranded viral RNA and activating TLR3, or CpG mimicking unmethylated bacterial DNA and activating TLR7/8), or small molecules agonists. Table 1 lists some known Pattern Recognition Receptors (PRR) inducing a Th1/Tc1 immune response and the corresponding natural PAMP or danger signal adjuvant inducing and supporting a Th1/Tc1 cytotoxic response. Of note, depending on the conditions, TLR2 and TLR5 may alternatively induce a non-cytotoxic Th2 T cell response. The Th1 adjuvants which are a danger signal listed in Table 1 are preferred as “Th1 adjuvant which is a danger signal” of the present invention.

TABLE 1

| | Pathogen | |
|---|---|---|
| | associated | |
| | molecular | Th1 adjuvants |
| PRR | pattern | Examples |
| STING | cGAMP, c-di-GMP, | STING agonists, e.g. STING activating |
| | c-di-AMP | cyclic dinucleotides, small molecules |
| RIG-I | dsRNA | poly (I:C), polyICLC, poly I:C12U, |
| | | polyI:C12C, RNA-based compounds, |
| | | small molecules |
| MD5 | dsRNA | poly (I:C), polylCLC, poly I:C12U, |
| | | polyI:C12C, RNA-based compounds, |
| | | small molecules |
| TLR3 | dsRNA | poly (I:C), polyICLC, ARNAX, poly |
| | | I:C12U, polyI:C12C, other dsRNA |
| | | mimicks, dsRNA, small molecules |
| TLR4 | Lipopolysaccharide | MPLA, GLA and formulations, small |
| | (LPS), RSV fusion | molecules |
| | protein | |
| TLR7 | ssRNA, | Imidazoquinolamines and guanosine |
| | imidazoquinolones | analogues, ssRNA, small molecules |
| TLR8 | ssRNA, | Imidazoquinolamines and guanosine |
| | imidazoquinolones | analogues, ssRNA, small molecules |
| TLR9 | CpG DANN | CpG variants, short DNA oligonucleotides |
| TLR2 | MALP-2, | Pam3CSK4, Pam2Cys, Pam3Cys, |
| | peptidoglycans, | lipoproteins, porins, toxins, small |
| | lipopeptides, | molecules |
| | lipotechoic acid | |
| TLR5 | Flagellin | Flagellin and derivatives, small molecules |

The Th1 adjuvant which is a danger signal can be administered temporally and/or spatially separate or together with the amplifying medicament. When administered temporally and spatially separate, the Th1 adjuvant which is a danger signal is preferably administered within 24 h after administration of the amplifying medicament. When administered temporally together with the amplifying medicament, the Th1 adjuvant which is a danger signal may be covalently or non-covalently attached to the cells used for the amplifying medicament or may be not attached to the cells, and may be in the same composition as the cells or in a separate composition, such as a separate vial or container.

In one embodiment of the present invention, the Th1 adjuvant which is a danger signal is recognized by a Pattern Recognition Receptor (PRR). In one embodiment of the present invention, the Th1 adjuvant which is a danger signal is or mimicks a pathogen-associated molecular pattern (PAMP).

In one embodiment of the present invention, the Th1 adjuvant which is a danger signal is administered systemically, locally, intravenously, subcutaneously, intraperitoneally, intratumorally or intradermally and/or by injection.

In one embodiment of a the Th1 adjuvant which is a danger signal for use of the present invention,
the Pattern Recognition Receptor (PRR) is selected from STING, RIG-I, MD5, TLR2, TLR3, TLR4, TLR5, TLR7, TLR8 and TLR9, and/or
the pathogen-associated molecular pattern (PAMP) is selected from cGAMP, c-di-GMP, c-di-AMP, dsRNA, MALP-2, a peptidoglycan, a lipopeptide, lipotechoic acid, lipopolysaccharide (LPS), RSV fusion protein, Flagellin, ssRNA, an imidazoquinolone, and a CpG poly- or oligodeoxynucleotide, and/or
the Th1 adjuvant which is a danger signal is selected from a STING agonist, preferably a STING activating cyclic dinucleotide or a small molecule, a RIG-I agonist, preferably selected from poly (I:C), polyICLC, poly I:C12U, polyL:C12C, an RNA-based RIG-I agonist, and a small molecule RIG-I agonist, an MD5 agonist preferably selected from poly (I:C), polyICLC, poly I:C12U, polyL:C12C, an RNA-based MD5 agonist, and a small molecule MD5 agonist, a TLR2 agonist, preferably selected from Pam3CSK4, Pam2Cys, Pam3Cys, lipoproteins, porins, toxins, and small molecule TLR2 agonists, a TLR3 agonist, preferably selected from poly (I:C), polyICLC, ARNAX, poly I:C12U, polyL:C12C, further dsRNA mimicks, dsRNA and small molecule TLR3 agonists, a TLR4 agonist, preferably selected from MPLA and GLA, and formulations thereof, small molecule TLR4 agonists, a TLR4 agonist, preferably selected from Flagellin and derivatives thereof and small molecule TLR5 agonists, a TLR7 agonist, preferably selected from an imidazoquinolamine, a guanosine analogue, ssRNA, and small molecule TLR7 agonists, a TLR8 agonist, preferably selected from an imidazoquinolamine, a guanosine analogue, ssRNA, and small molecule TLR8 agonists, a TLR9 agonist, preferably selected from a CpG poly- or oligodeoxynucleotide or a variant thereof, and a short DNA oligonucleotide.

A fusion protein for use according to the invention (and any other medicament or therapeutic agent mentioned herein) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Fusion proteins for use of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The fusion protein needs not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an fusion protein for use of the invention (when used alone or in combination with one or more other additional therapeutic agents) will depend on the type of disease to be treated, the type of fusion protein, the severity and course of the disease, whether the fusion protein is administered for preventive or therapeutic purposes, previous therapy, the patient's clinical history and response to the fusion protein, and the discretion of the attending physician. The fusion protein is suitably administered to the patient at one time or over a series of treatments. Depending on the type and severity of the disease, about 1 μg/kg to 15 mg/kg (e.g. 0.1 mg/kg-10 mg/kg) of fusion protein for use can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion protein would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

The terms “administered in combination with”, “co-administration”, “co-administering”, “combination therapy”, “administered with” or “combination treatment” refer to the administration of the peptide-MHC-I-antibody fusion protein as described herein, and the antibody-peptide fusion protein as well as the class I MHC expressing, antigen presenting cell loaded with the virus-derived peptide and the adjuvant which supports a Th1 mediated response as described herein, as separate formulations/applications to the same patient.

In one embodiment,

(a) the peptide-MHC-I-antibody fusion protein, and/or

(b) the antibody-peptide fusion protein, and/or

(c) the class I MHC expressing, antigen presenting cell loaded with the virus-derived peptide and the Th1 adjuvant, which is a danger signal

are administered as separate formulations and in different application schemes.

The amount of co-administration and the timing of co-administration will depend on the type (species, gender, age, weight, etc.) and condition of the patient being treated and the severity of the disease or condition being treated.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

An “effective amount” of an agent, e.g., a pharmaceutical formulation, refers to an amount effective, at dosages and for periods of time necessary, to achieve the desired therapeutic or prophylactic result.

As used herein, the term “patient”, “subject” or “individual” preferably refers to a human in need of treatment of cancer or a viral infection. However, the term “patient”, “subject” or “individual” may also refer to non-human animals, e.g. mammals such as mice, dogs, cats, horses, cows, pigs, sheep and non-human primates, among others, that are in need of treatment.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants. The amino acid sequence of an exemplary human IL-2 is shown in SEQ ID NO: 2. Unprocessed human IL-2 additionally comprises an N-terminal 20 amino acid signal peptide having the sequence of SEQ ID NO: 3 (MYRMQLLSCIALSLALVTNS, leader sequence), which is absent in the mature IL-2 molecule.

The term “IL-2 mutant” or “mutant IL-2 polypeptide” as used herein is intended to encompass any mutant forms of various forms of the IL-2 molecule including full-length IL-2, truncated forms of IL-2 and forms where IL-2 is linked to another molecule such as by fusion or chemical conjugation. “Full-length” when used in reference to IL-2 is intended to mean the mature, natural length IL-2 molecule. For example, full-length human IL-2 refers to a molecule that has 133 amino acids (see e.g. SEQ ID NO: 2). The various forms of IL-2 mutants are characterized in having a at least one amino acid mutation affecting the interaction of IL-2 with CD25. This mutation may involve substitution, deletion, truncation or modification of the wild-type amino acid residue normally located at that position. Mutants obtained by amino acid substitution are preferred. Unless otherwise indicated, an IL-2 mutant may be referred to herein as an IL-2 mutant peptide sequence, an IL-2 mutant polypeptide, IL-2 mutant protein or IL-2 mutant analog. Preferably, the IL-2 mutant is a IL-2 variant with extended half-life in the patient (see also below).

As used herein, “complexed IL-2” or “IL-2cx” is understood as IL-2 protein which is non-covalently bound to a binding molecule, in particular an antibody or antibody fragment which is blocking its binding to the high affinity IL-2 receptor chain (CD25). In a preferred embodiment, the antibody is humanized or human. In a preferred embodiment, IL-2 is human IL-2, more preferably human wildtype IL-2. IL-2 may be synthesized synthetically or recombinantly, using an adequate host, more preferably IL-2 is prepared recombinantly. In a preferred embodiment, binding of complexed IL-2 to CD25 is reduced by at least 35%, more preferably at least 50%, even more preferably at least 75%, most preferably at least 90% or 95%, such as 100%, as compared to binding of uncomplexed IL-2 to CD25. Binding to CD25 may be determined as Kd value using surface plasmon resonance spectroscopy, which is known to a person skilled in the art. Further details on compled IL-2 is described below and in EP 18213650 (see page 4, last paragraph to page 5, 4^{th} paragraph as well as FIGS. 1 to 3 and Examples 1 to 3).

An IL-2 mutein is understood as IL-2 protein, in particular human IL-2 protein, which is mutated as compared to the wt IL-2 protein, and which binds to CD122, but exhibits reduced binding to CD25 as compared to the wt IL-2 protein. In a preferred embodiment, binding to CD25 is reduced by at least 35%, more preferably by at least 50%, even more preferably by at least 75%, most preferably by at least 90% or 95%, such as by 100%, as compared to binding of wt IL-2 protein to CD25. Binding to CD25 may be determined as determining affinity, expressed as Kd value, in particular using surface plasmon resonance spectroscopy, which is known to persons skilled in the art. Moreover, binding to CD122 may be determined using surface plasmon resonance spectroscopy. In a preferred embodiment, binding of the IL-2 mutein to CD122 is at least 10%, preferably at least 35%, more preferably at least 50%, even more preferably at least 90% or 95%, such as 100% or more, of the binding of the wt IL-2 protein to CD122. A particularly preferred IL-2 mutein for use in the present invention is disclosed in Carmenate, Journal of Immunology 2013. In particular, a R38, F42, Y45, E62 mutein of wt IL-2, preferably R38A, F42A, Y45A, E62A mutein of wt IL-2 may be used, which mutein may further comprise a C125 mutation, such as a C125S mutation. Another particularly preferred IL-2 mutein which may be used according to the present invention is described in Klein et al. 2014. In a more preferred embodiment of the medicament for use or method of the invention, the complexed interleukin 2 (IL-2cx), complexed interleukin 15 (IL-15cx), complexed interleukin 4 (IL-4cx), complexed interleukin 7 (IL-7cx), or IL-2 mutein is administered repeatedly, in particular 1, 2, 3, 4, 5, 6, 7, 8, 9, 10, 11, 12, 13 or 14 times, even more preferably wherein the complexed interleukin 2 (IL-2cx), complexed interleukin 15 (IL-15cx), complexed interleukin 4 (IL-4cx), complexed interleukin 7 (IL-7cx), or IL-2 mutein is administered every 1 or 2 days, and/or is administered repeatedly during 5 days to 1 month, even more preferably during 1 to 2 weeks.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to CD25. Amino acid sequence deletions and insertions include amino- and/or carboxy-terminal deletions and insertions of amino acids. An example of a terminal deletion is the deletion of the alanine residue in position 1 of full-length human IL-2. Preferred amino acid mutations are amino acid substitutions. For the purpose of altering e.g. the binding characteristics of an IL-2 polypeptide, non-conservative amino acid substitutions, i.e. replacing one amino acid with another amino acid having different structural and/or chemical properties, are particularly preferred. Preferred amino acid substitutions include replacing a hydrophobic by a hydrophilic amino acid. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

As used herein, a “wild-type” form of IL-2 is a form of IL-2 that is otherwise the same as the mutant IL-2 polypeptide except that the wild-type form has a wild-type amino acid at each amino acid position of the mutant IL-2 polypeptide. For example, if the IL-2 mutant is the full-length IL-2 (i.e. IL-2 not fused or conjugated to any other molecule), the wild-type form of this mutant is full-length native IL-2. If the IL-2 mutant is a fusion between IL-2 and another polypeptide encoded downstream of IL-2 (e.g. an antibody chain) the wild-type form of this IL-2 mutant is IL-2 with a wild-type amino acid sequence fused to the same downstream polypeptide. Furthermore, if the IL-2 mutant is a truncated form of IL-2 (the mutated or modified sequence within the non-truncated portion of IL-2) then the wild-type form of this IL-2 mutant is a similarly truncated IL-2 that has a wild-type sequence. For the purpose of comparing IL-2 receptor binding affinity or biological activity of various forms of IL-2 mutants to the corresponding wild-type form of IL-2, the term wild-type encompasses forms of IL-2 comprising one or more amino acid mutation that does not affect IL-2 receptor binding compared to the naturally occurring, native IL-2, such as e.g. a substitution of cysteine at a position corresponding to residue 125 of human IL-2 to alanine. In some embodiments wild-type IL-2 for the purpose of the present invention comprises the amino acid substitution C125A (see SEQ ID NO: 3). In certain embodiments according to the invention the wild-type IL-2 polypeptide to which the mutant IL-2 polypeptide is compared comprises the amino acid sequence of SEQ ID NO: 1. In other embodiments the wild-type IL-2 polypeptide to which the mutant IL-2 polypeptide is compared comprises the amino acid sequence of SEQ ID NO: 3.

The term “CD25” or “α-subunit of the IL-2 receptor” as used herein, refers to any native CD25 from any vertebrate source, including mammals such as primates (e.g. humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g. splice variants or allelic variants. In certain embodiments CD25 is human CD25. The amino acid sequence of an exemplary human CD25 (with signal sequence, Avi-tag and His-tag) is shown in SEQ ID NO: 278.

The term “high-affinity IL-2 receptor” as used herein refers to the heterotrimeric form of the IL-2 receptor, consisting of the receptor γ-subunit (also known as common cytokine receptor γ-subunit, γc, or CD132), the receptor β-subunit (also known as CD122 or p70) and the receptor α-subunit (also known as CD25 or p55). The term “intermediate-affinity IL-2 receptor” by contrast refers to the IL-2 receptor including only the γ-subunit and the β-subunit, without the α-subunit (for a review see e.g. Olejniczak and Kasprzak, Med Sci Monit 14, RA179-189 (2008)).

### (b) Detailed Description of the Embodiments of the Invention

The invention relates to a peptide-MHC-I-antibody fusion protein, wherein the peptide-MHC-I-antibody fusion protein comprises
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule,
   for use as a therapeutic medicament,
   wherein the peptide-MHC-I-antibody fusion protein is to be administered to a patient, wherein a cellular cytotoxic immune response towards the virus-derived peptide has been induced in the patient and wherein the induced immune response towards the virus-derived peptide has been amplified in the patient by an amplifying medicament.

In one embodiment, the cellular cytotoxic immune response towards the virus-derived peptide has been induced by vaccination with an inducing medicament and/or by infection with a virus, thereby activating T cells against the virus-derived peptide

Additionally, or alternatively, the amplifying medicament comprises a class I MHC expressing, antigen presenting cell, wherein the MHC-I is bound to the virus-derived peptide, and a Th1 adjuvant, which is a danger signal.

In one embodiment the inducing medicament comprises
(a) lymphotactin (XCL1) or a variant or fragment thereof, specifically binding to XCR1, and
(b) the virus-derived peptide, wherein the peptide is covalently coupled to XCL1 or the variant or fragment thereof.

In another embodiment inducing medicament comprises
(a) an antibody or a variant or fragment thereof, specifically binding to chemokine (C motif) receptor 1 (XCR1), and
(b) the virus-derived peptide, wherein the peptide is covalently coupled to said antibody or the variant or fragment thereof.

Another aspect of the invention is a kit of parts comprising an amplifying medicament and a therapeutic medicament,
the amplifying medicament comprising
(a) a class I MHC expressing, antigen presenting cell, wherein the MHC-I is bound to the virus-derived peptide, and
(b) a Th1 adjuvant, which is a danger signal, wherein the amplifying medicament amplifies an immune response towards a virus-derived peptide in a patient, and
the therapeutic medicament comprising
(i) an antibody specifically binding to a target cell, and
(ii) a polypeptide comprising in N- to C-terminal direction the virus-derived peptide, a β2-microglobulin and the extracellular domains α1, α2, and α3 of a class I MHC molecule.

The kit may further comprise an inducing medicament inducing a cellular cytotoxic immune response towards the virus-derived peptide in a patient.

The kit may be used as a medicament, wherein the therapeutic medicament is to be administered to a patient with amplified immune response towards the virus-derived peptide, wherein the amplifying medicament amplifies an immune response towards a virus-derived peptide in a patient and wherein the inducing medicament induces the cellular cytotoxic immune response towards the virus-derived peptide in the patient by vaccination, particularly wherein the inducing medicament is as defined above.

Another aspect of the invention is a kit of parts comprising an inducing medicament and an amplifying medicament,
the inducing medicament being defined as above, and
the amplifying medicament being defined as above, for use in a method of making a patient susceptible for a treatment with the peptide-MHC-I-antibody fusion protein comprising
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule.

In one embodiment of the invention the inducing medicament has been or is administered in combination with a Th1 adjuvant, which is a danger signal.

In another embodiment of the invention interleukin-2 (IL-2) or IL-2 variant with extended half-life in the patient has been or is administered after the administration of the class I MHC expressing, antigen presenting cell.

In still another embodiment of the invention the Th1 adjuvant is polyinosinic:polycytidylic acid (poly I:C).

Yet, in another embodiment of the invention the class I MHC expressing, antigen presenting cell, and the adjuvant has been or is administered 3 days to 14 days, preferably 4 days to 10 days, after the T cells of patient were induced against said virus-derived peptide.

In another embodiment of the invention the therapeutic medicament is administered when the level of virus-derived peptide-specific CD8+ T cells is elevated as a result of the administration of the amplifying medicament, preferably wherein the therapeutic medicament is administered within 29 days, particularly 20 days, especially 14 days, from the administration of the amplifying medicament. More preferably, the therapeutic medicament is administered shortly after the amplification, such as within 3, 2 or 1 day from the administration of the amplifying medicament or immediately thereafter.

In still another embodiment of the invention the antibody comprised in the peptide-MHC-I-antibody fusion protein specifically binds to a target cell selected from the group of cancer cells and virus-infected cells.

In still another embodiment of the invention, the peptide-MHC-I-antibody fusion protein for use, or the kit of parts is for use in treating cancer or a viral infection.

### Peptide-MHC-I-Antibody Fusion Protein

The following paragraphs include embodiments further defining the peptide-MHC-I-antibody fusion protein.

### Antibody of (i)

In one embodiment the antibody comprised in the peptide-MHC-I-antibody fusion protein is a full length antibody.

In one embodiment the antibody comprised in the peptide-MHC-I-antibody fusion protein is a monospecific antibody.

In one embodiment the antibody comprised in the peptide-MHC-I-antibody fusion protein is a bivalent antibody.

In one embodiment the antibody comprised in the peptide-MHC-I-antibody fusion protein is a multispecific antibody. In one embodiment the antibody comprised in the peptide-MHC-I-antibody fusion protein is a bispecific antibody.

Multispecific antibodies are monoclonal antibodies that have binding specificities for at least two different sites, i.e., different epitopes on different antigens or different epitopes on the same antigen. In certain embodiments, the multispecific antibody has three or more binding specificities. In certain embodiments, one of the binding specificities is for an antigen expressed on the target cell and the other (two or more) specificity is for any other antigen. In certain embodiments, bispecific antibodies may bind to two (or more) different epitopes of the antigen expressed on the target cell.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies,” or DVD-Ig are also included herein (see, e.g. WO 2001/77342 and WO 2008/024715). Other examples of multispecific antibodies with three or more antigen binding sites can be found in WO 2010/115589, WO 2010/112193, WO 2010/136172, WO2010/145792, and WO 2013/026831. The bispecific antibody or antigen binding fragment thereof also includes a “Dual Acting Fab” or “DAF” comprising an antigen binding site that binds to an antigen expressed on the target cell as well as another different antigen, or two different epitopes of the antigen expressed on the target cell (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one embodiment, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

In one embodiment the antibody comprised in the peptide-MHC-I-antibody fusion protein comprises immunoglobulin constant regions of one or more immunoglobulin classes. Immunoglobulin classes include IgG, IgM, IgA, IgD, and IgE isotypes and, in the case of IgG and IgA, their subtypes.

In one embodiment the antibody comprised in the peptide-MHC-I-antibody fusion protein is of IgG or IgE class. In one embodiment the antibody comprised in the peptide-MHC-I-antibody fusion protein is of IgG1, IgG2, IgG3 or IgG4 isotype. In one embodiment the antibody comprised in the peptide-MHC-I-antibody fusion protein is of human IgG1 or human IgG2 isotype.

In one embodiment the antibody comprised in the peptide-MHC-I-antibody fusion protein is of human IgG1 or human IgG2 isotype with the mutations L234A and L235A (numbering according to the EU index of Kabat).

In one embodiment the antibody comprised in the peptide-MHC-I-antibody fusion protein is of human IgG1 or human IgG2 isotype with the mutations D265A and N297A (numbering according to the EU index of Kabat).

In one embodiment the antibody comprised in the peptide-MHC-I-antibody fusion protein is of human IgG1 or human IgG2 isotype with the mutation P329G (numbering according to the EU index of Kabat).

In one embodiment the antibody comprised in the peptide-MHC-I-antibody fusion protein is of human IgG1 or human IgG2 isotype with the mutations L234A, L235A and P329G.

In one embodiment the antibody comprised in the peptide-MHC-I-antibody fusion protein comprises amino acid mutations in the CH3 domains that support heterodimerization of the antibody heavy chains. In one embodiment the peptide-MHC-I-antibody fusion protein, which comprises exactly one polypeptide of (ii) comprises amino acid mutations in the CH3 domains that support heterodimerization of the antibody heavy chains.

Several approaches for CH3-modifications in order to support heterodimerization have been described, for example in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291, which are herein included by reference. Typically, in the approaches known in the art, the CH3 domain of the first heavy chain and the CH3 domain of the second heavy chain are both engineered in a complementary manner so that the heavy chain comprising one engineered CH3 domain can no longer homodimerize with another heavy chain of the same structure (e.g. a CH3-engineered first heavy chain can no longer homodimerize with another CH3-engineered first heavy chain; and a CH3-engineered second heavy chain can no longer homodimerize with another CH3-engineered second heavy chain). Thereby the heavy chain comprising one engineered CH3 domain is forced to heterodimerize with another heavy chain comprising the CH3 domain, which is engineered in a complementary manner. For this embodiment of the invention, the CH3 domain of the first heavy chain and the CH3 domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g. for sterical reasons).

In one embodiment the antibody of (i) comprises a first heavy chain including a CH3 domain and a second heavy chain including a CH3 domain, the CH3 domains of the first and second heavy chain are engineered by the so-called “knob-into-hole” technology, which is described in detail providing several examples in e.g. WO 96/027011, Ridgway, J. B., et al., Protein Eng. 9 (1996) 617-621; Merchant, A. M., et al., Nat. Biotechnol. 16 (1998) 677-681; and WO 98/050431, which are herein included by reference. In the “knob-into-hole” technology, within the interface formed between the CH3 domain of the first heavy chain and the CH3 domain of the second heavy chain in the tertiary structure of the antibody, particular amino acids on each CH3 domain are engineered to produce a protuberance (“knob”) in the CH3 domain of one heavy chain and a cavity (“hole”) in the CH3 domain of the other heavy chain, respectively. In the tertiary structure of the antibody the introduced protuberance in the CH3 domain of the one heavy chain is positionable in the introduced cavity in the CH3 domain of the other heavy chain. Each of the heavy chains can comprise the “knob” in its CH3 domain while the other heavy chain comprises the “hole” in its CH3 domain.

In one embodiment the antibody of (i) comprises up to five mutations in each CH3 domain that support heterodimerization of the antibody heavy chains.

In one embodiment in the tertiary structure of the antibody of (i) the CH3 domain of a first heavy chain and the CH3 domain of a second heavy chain form an interface that is located between the respective antibody CH3 domains, wherein the respective amino acid sequences of the CH3 domain of the first heavy chain and the CH3 domain of the second heavy chain each comprise a set of amino acids that is located within said interface in the tertiary structure of the antibody,
(i) wherein from the set of amino acids that is located in the interface in the CH3 domain of one heavy chain at least one amino acid residue is substituted by an amino acid residue having a larger side chain volume than the original amino acid residue, thereby generating a protuberance within the interface, wherein the protuberance is located in the CH3 domain of the one heavy chain, and wherein the protuberance is positionable in a cavity located in the CH3 domain of the other heavy chain within the interface; and
(ii) wherein from the set of amino acids that is located in the interface in the CH3 domain of the other heavy chain at least one amino acid residue is substituted by an amino acid residue having a smaller side chain volume than the original amino acid residue, thereby generating a cavity within the interface, wherein the cavity is located in the CH3 domain of the other heavy chain, and wherein in the cavity the protuberance within the interface located in the CH3 domain of the one heavy chain is positionable.

In other words, this embodiment relates to a peptide-MHC-I-antibody fusion protein, wherein the CH3 domain of one heavy chain and the CH3 domain of the other heavy chain of the antibody of (i) each meet at an interface which comprises an original interface between the antibody CH3 domains; wherein said interface is altered to promote the formation of the antibody, wherein the alteration is characterized in that:
(iii) the CH3 domain of one heavy chain is altered, so that within the original interface the CH3 domain of one heavy chain that meets the original interface of the CH3 domain of the other heavy chain within the antibody an amino acid residue is replaced with an amino acid residue having a larger side chain volume, thereby generating a protuberance within the interface of the CH3 domain of one heavy chain which is positionable in a cavity within the interface of the CH3 domain of the other heavy chain; and
(iv) the CH3 domain of the other heavy chain is altered, so that within the original interface of the second CH3 domain that meets the original interface of the first CH3 domain within the antibody an amino acid residue is replaced with an amino acid residue having a smaller side chain volume, thereby generating a cavity within the interface of the second CH3 domain within which a protuberance within the interface of the first CH3 domain is positionable.

The antibody according to this embodiment is herein also referred to as “CH3(KiH)-engineered antibody” (wherein the abbreviation “KiH” stands for the “knob-into-hole technology”).

In one embodiment said amino acid residue having a larger side chain volume than the original amino acid residue is selected from R, F, Y and W; and wherein said amino acid residue having a smaller side chain volume than the original amino acid residue is selected from A, S, T and V.

In one embodiment in the CH3 domain of the one heavy chain the amino acid T at position 366 is substituted by W; and in the CH3 domain of the other heavy chain the amino acid T at position 366 is substituted by S, the amino acid L at position 368 is substituted by A and the amino acid Y at position 407 is substituted by V (numbering according to EU index of Kabat).

In one embodiment in the CH3 domain of the one heavy chain the amino acid T at position 366 is substituted by W, the amino acid R at position 409 is substituted by D, and the amino acid K at position 370 is substituted by E; and in the CH3 domain of the other heavy chain the amino acid T at position 366 is substituted by S, the amino acid L at position 368 is substituted by A and the amino acid Y at position 407 is substituted by V, the amino acid D at position 399 is substituted by K, and the amino acid E at position 357 is substituted by K (numbering according to EU index of Kabat).

In addition to the engineering of the CH3 domains of the first and second heavy chain by the “knob-into-hole” technology, the introduction of a disulfide bridge further stabilizes the heterodimers (Atwell, S., et al., J. Mol. Biol. 270 (1997) 26-35; Merchant, A. M., et al., Nature Biotech. 16 (1998) 677-681). Thereby the additional introduction of a disulfide bridge further increases the yield of the antibody according to the invention.

In one embodiment from the set of amino acids that is located in the interface in the CH3 domain of the one heavy chain a first amino acid is substituted by cysteine; and from the set of amino acids that is located in the interface in the CH3 domain of the other heavy chain a second amino acid is substituted by cysteine, wherein the second amino acid is facing the first amino acid within the interface; such that a disulfide bridge between the CH3 domain of the one heavy chain and the CH3 domain of the other heavy chain can be formed via the introduced cysteine residues.

In one embodiment in the CH3 domain of the one heavy chain either the amino acid E at position 356 or the amino acid S at position 354 is substituted by C, and in the CH3 domain of the other heavy chain the amino acid Y at position 349 is substituted by C (numbering according to EU index of Kabat).

In one embodiment within said CH3(KiH)-engineered antibody said amino acid residue having a larger side chain volume than the original amino acid residue is selected from R, F, Y and W; and from the set of amino acids that is located in the interface in the CH3 domain of the one heavy chain a first amino acid is substituted by cysteine; and from the set of amino acids that is located in the interface in the CH3 domain of the other heavy chain a second amino acid is substituted by cysteine, wherein the second amino acid is facing the first amino acid within the interface; such that a disulfide bridge between the CH3 domain of the one heavy chain and the CH3 domain of the other heavy chain can be formed via the introduced cysteine residues.

In one embodiment within said CH3(KiH)-engineered antibody said amino acid residue having a smaller side chain volume than the original amino acid residue is selected from A, S, T and V; and from the set of amino acids that is located in the interface in the CH3 domain of the one heavy chain a first amino acid is substituted by cysteine; and from the set of amino acids that is located in the interface in the CH3 domain of the other heavy chain a second amino acid is substituted by cysteine, wherein the second amino acid is facing the first amino acid within the interface; such that a disulfide bridge between the CH3 domain of the one heavy chain and the CH3 domain of the other heavy chain can be formed via the introduced cysteine residues.

In one embodiment within said CH3(KiH)-engineered antibody said amino acid residue having a larger side chain volume than the original amino acid residue is selected from R, F, Y and W; said amino acid residue having a smaller side chain volume than the original amino acid residue is selected from A, S, T and V; and from the set of amino acids that is located in the interface in the CH3 domain of the one heavy chain a first amino acid is substituted by cysteine; and from the set of amino acids that is located in the interface in the CH3 domain of the other heavy chain a second amino acid is substituted by cysteine, wherein the second amino acid is facing the first amino acid within the interface; such that a disulfide bridge between the CH3 domain of the one heavy chain and the CH3 domain of the other heavy chain can be formed via the introduced cysteine residues.

In one embodiment within said CH3(KiH)-engineered antibody said amino acid residue having a larger side chain volume than the original amino acid residue is selected from R, F, Y and W; and in the CH3 domain of the one heavy chain (the heavy chain comprising the “knob”) either the amino acid E at position 356 (numbering according to EU index of Kabat) or the amino acid S at position 354 (numbering according to EU index of Kabat) is substituted by C and in the CH3 domain of the other heavy chain (the heavy chain comprising the “hole”) the amino acid Y at position 349 (numbering according to EU index of Kabat) is substituted by C.

In one embodiment within said CH3(KiH)-engineered antibody said amino acid residue having a smaller side chain volume than the original amino acid residue is selected from A, S, T and V; and in the CH3 domain of the one heavy chain (the heavy chain comprising the “knob”) either the amino acid E at position 356 (numbering according to EU index of Kabat) or the amino acid S at position 354 (numbering according to EU index of Kabat) is substituted by C and in the CH3 domain of the other heavy chain (the heavy chain comprising the “hole”) the amino acid Y at position 349 (numbering according to EU index of Kabat) is substituted by C.

In one embodiment within said CH3(KiH)-engineered antibody said amino acid residue having a larger side chain volume than the original amino acid residue is selected from R, F, Y and W; and said amino acid residue having a smaller side chain volume than the original amino acid residue is selected from A, S, T and V; and in the CH3 domain of the one heavy chain (the heavy chain comprising the “knob”) either the amino acid E at position 356 (numbering according to EU index of Kabat) or the amino acid S at position 354 (numbering according to EU index of Kabat) is substituted by C and in the CH3 domain of the other heavy chain (the heavy chain comprising the “hole”) the amino acid Y at position 349 (numbering according to EU index of Kabat) is substituted by C.

In one embodiment within said CH3(KiH)-engineered antibody, in the CH3 domain of the one heavy chain (the heavy chain comprising the “knob”) the amino acid T at position 366 (numbering according to EU index of Kabat) is substituted by W and either the amino acid E at position 356 (numbering according to EU index of Kabat) or the amino acid S at position 354 (numbering according to EU index of Kabat) is substituted by C; and in the CH3 domain of the other heavy chain (the heavy chain comprising the “hole”) the amino acid T at position 366 (numbering according to EU index of Kabat) is substituted by S, the amino acid L at position 368 (numbering according to EU index of Kabat) is substituted by A, the amino acid Y at position 407 (numbering according to EU index of Kabat) is substituted by V and the amino acid Y at position 349 (numbering according to EU index of Kabat) is substituted by C.

In one embodiment within said CH3(KiH)-engineered antibody, in the CH3 domain of the one heavy chain (the heavy chain comprising the “knob”) the amino acid T at position 366 (numbering according to EU index of Kabat) is substituted by W and the amino acid Y at position 349 (numbering according to EU index of Kabat) is substituted by C; and in the CH3 domain of the other heavy chain (the heavy chain comprising the “hole”) the amino acid T at position 366 (numbering according to EU index of Kabat) is substituted by S, the amino acid L at position 368 (numbering according to EU index of Kabat) is substituted by A, the amino acid Y at position 407 (numbering according to EU index of Kabat) is substituted by V and either the amino acid E at position 356 (numbering according to EU index of Kabat) or the amino acid S at position 354 (numbering according to EU index of Kabat) is substituted by C.

In one embodiment within said CH3(KiH)-engineered antibody, in the CH3 domain of the one heavy chain (the heavy chain comprising the “knob”) the amino acid T at position 366 (numbering according to EU index of Kabat) is substituted by W, the amino acid R at position 409 (numbering according to EU index of Kabat) is substituted by D, the amino acid K at position 370 (numbering according to EU index of Kabat) is substituted by E and either the amino acid E at position 356 (numbering according to EU index of Kabat) or the amino acid S at position 354 (numbering according to EU index of Kabat) is substituted by C; and in the CH3 domain of the other heavy chain (the heavy chain comprising the “hole”) the amino acid T at position 366 (numbering according to EU index of Kabat) is substituted by S, the amino acid L at position 368 (numbering according to EU index of Kabat) is substituted by A and the amino acid Y at position 407 (numbering according to EU index of Kabat) is substituted by V, the amino acid D at position 399 (numbering according to EU index of Kabat) is substituted by K, the amino acid E at position 357 (numbering according to EU index of Kabat) is substituted by K and the amino acid Y at position 349 (numbering according to EU index of Kabat) is substituted by C.

In one embodiment within said CH3(KiH)-engineered antibody, in the CH3 domain of the one heavy chain (the heavy chain comprising the “knob”) the amino acid T at position 366 (numbering according to EU index of Kabat) is substituted by W, the amino acid R at position 409 (numbering according to EU index of Kabat) is substituted by D, the amino acid K at position 370 (numbering according to EU index of Kabat) is substituted by E and the amino acid Y at position 349 (numbering according to EU index of Kabat) is substituted by C; and in the CH3 domain of the other heavy chain (the heavy chain comprising the “hole”) the amino acid T at position 366 (numbering according to EU index of Kabat) is substituted by S, the amino acid L at position 368 (numbering according to EU index of Kabat) is substituted by A and the amino acid Y at position 407 (numbering according to EU index of Kabat) is substituted by V, the amino acid D at position 399 (numbering according to EU index of Kabat) is substituted by K, the amino acid E at position 357 (numbering according to EU index of Kabat) is substituted by K and either the amino acid E at position 356 (numbering according to EU index of Kabat) or the amino acid S at position 354 (numbering according to EU index of Kabat) is substituted by C.

Apart from the “knob-into-hole technology” other techniques for modifying the CH3 domains of the heavy chains of an antibody to enforce heterodimerization are known in the art. These technologies, especially the ones described in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291 are contemplated herein as alternatives to the “knob-into-hole technology” in combination with an antibody according to the invention.

Accordingly, this embodiment relates to a peptide-MHC-I-antibody fusion protein comprising an antibody, wherein in the tertiary structure of the antibody the CH3 domain of the first heavy chain and the CH3 domain of the second heavy chain form an interface that is located between the respective antibody CH3 domains, wherein the respective amino acid sequences of the CH3 domain of the first heavy chain and the CH3 domain of the second heavy chain each comprise a set of amino acids that is located within said interface in the tertiary structure of the antibody, wherein from the set of amino acids that is located in the interface in the CH3 domain of one heavy chain a first amino acid is substituted by a positively charged amino acid and from the set of amino acids that is located in the interface in the CH3 domain of the other heavy chain a second amino acid is substituted by a negatively charged amino acid. The antibody according to this embodiment is herein also referred to as “CH3(+/−)-engineered antibody” (wherein the abbreviation “+/−” stands for the oppositely charged amino acids that were introduced in the respective CH3 domains).

In one embodiment of said CH3(+/−)-engineered antibody according to the invention the positively charged amino acid is selected from K, R and H; and the negatively charged amino acid is selected from E or D.

In one embodiment of said CH3(+/−)-engineered antibody the positively charged amino acid is selected from K and R; and the negatively charged amino acid is selected from E or D.

In one embodiment of said CH3(+/−)-engineered antibody the positively charged amino acid is K; and the negatively charged amino acid is E.

In one embodiment of said CH3(+/−)-engineered antibody in the CH3 domain of one heavy chain the amino acid R at position 409 (numbering according to EU index of Kabat) is substituted by D and the amino acid K at position 370 (numbering according to EU index of Kabat) is substituted by E; and in the CH3 domain of the other heavy chain the amino acid D at position 399 (numbering according to EU index of Kabat) is substituted by K and the amino acid E at position 357 (numbering according to EU index of Kabat) is substituted by K.

In one embodiment within the antibody comprised in the peptide-MHC-I-antibody fusion protein the approach described in WO2013/157953 is used to support heterodimerization of the first heavy chain and the second heavy chain of the antibody. In one embodiment of said CH3-engineered antibody in the CH3 domain of one heavy chain the amino acid T at position 366 (numbering according to EU index of Kabat) is substituted by K; and in the CH3 domain of the other heavy chain the amino acid L at position 351 (numbering according to EU index of Kabat) is substituted by D. In another embodiment of said CH3-engineered antibody, in the CH3 domain of one heavy chain the amino acid T at position 366 (numbering according to EU index of Kabat) is substituted by K and the amino acid L at position 351 (numbering according to EU index of Kabat) is substituted by K; and in the CH3 domain of the other heavy chain the amino acid L at position 351 (numbering according to EU index of Kabat) is substituted by D.

In another embodiment of said CH3-engineered antibody, in the CH3 domain of one heavy chain the amino acid T at position 366 (numbering according to EU index of Kabat) is substituted by K and the amino acid L at position 351 (numbering according to EU index of Kabat) is substituted by K; and in the CH3 domain of the other heavy chain the amino acid L at position 351 (numbering according to EU index of Kabat) is substituted by D. Additionally at least one of the following substitutions is comprised in the CH3 domain of the other heavy chain: the amino acid Y at position 349 (numbering according to EU index of Kabat) is substituted by E, the amino acid Y at position 349 (numbering according to EU index of Kabat) is substituted by D and the amino acid L at position 368 (numbering according to EU index of Kabat) is substituted by E. In one embodiment the amino acid L at position 368 (numbering according to EU index of Kabat) is substituted by E.

In one embodiment within the antibody comprised in the peptide-MHC-I-antibody fusion protein the approach described in WO2012/058768 is used to support heterodimerization of the first heavy chain and the second heavy chain of the antibody. In one embodiment of said CH3-engineered antibody, in the CH3 domain of one heavy chain the amino acid L at position 351 (numbering according to EU index of Kabat) is substituted by Y and the amino acid Y at position 407 (numbering according to EU index of Kabat) is substituted by A; and in the CH3 domain of the other heavy chain the amino acid T at position 366 (numbering according to EU index of Kabat) is substituted by A and the amino acid K at position 409 (numbering according to EU index of Kabat) is substituted by F. In another embodiment, in addition to the aforementioned substitutions, in the CH3 domain of the other heavy chain at least one of the amino acids at positions 411 (originally T), 399 (originally D), 400 (originally S), 405 (originally F), 390 (originally N) and 392 (originally K) is substituted. Preferred substitutions are:
(a) substituting the amino acid T at position 411 (numbering according to EU index of Kabat) by an amino acid selected from N, R, Q, K, D, E and W;
(b) substituting the amino acid D at position 399 (numbering according to EU index of Kabat) by an amino acid selected from R, W, Y, and K;
(c) substituting the amino acid S at position 400 (numbering according to EU index of Kabat) by an amino acid selected from E, D, R and K;
(d) substituting the amino acid F at position 405 (numbering according to EU index of Kabat) by an amino acid selected from I, M, T, S, V and W;
(e) substituting the amino acid N at position 390 (numbering according to EU index of Kabat) by an amino acid selected from R, K and D; and
(f) substituting the amino acid K at position 392 (numbering according to EU index of Kabat) by an amino acid selected from V, M, R, L, F and E.

In another embodiment of said CH3-engineered antibody (engineered according to WO2012/058768), in the CH3 domain of one heavy chain the amino acid L at position 351 (numbering according to EU index of Kabat) is substituted by Y and the amino acid Y at position 407 (numbering according to EU index of Kabat) is substituted by A; and in the CH3 domain of the other heavy chain the amino acid T at position 366 (numbering according to EU index of Kabat) is substituted by V and the amino acid K at position 409 (numbering according to EU index of Kabat) is substituted by F. In another embodiment of said CH3-engineered antibody, in the CH3 domain of one heavy chain the amino acid Y at position 407 (numbering according to EU index of Kabat) is substituted by A; and in the CH3 domain of the other heavy chain the amino acid T at position 366 (numbering according to EU index of Kabat) is substituted by A and the amino acid K at position 409 (numbering according to EU index of Kabat) is substituted by F. In said last aforementioned embodiment, in the CH3 domain of said other heavy chain the amino acid K at position 392 (numbering according to EU index of Kabat) is substituted by E, the amino acid T at position 411 (numbering according to EU index of Kabat) is substituted by E, the amino acid D at position 399 (numbering according to EU index of Kabat) is substituted by R and the amino acid S at position 400 (numbering according to EU index of Kabat) is substituted by R.

In one embodiment within the antibody comprised in the peptide-MHC-I-antibody fusion protein the approach described in WO 2011/143545 is used to support heterodimerization of the first heavy chain and the second heavy chain of the antibody. In one embodiment of said CH3-engineered antibody, amino acid modifications in the CH3 domains of both heavy chains are introduced at positions 368 and/or 409.

In one embodiment within the antibody comprised in the peptide-MHC-I-antibody fusion protein the approach described in WO 2011/090762 is used to support heterodimerization of the first heavy chain and the second heavy chain of the antibody. WO 2011/090762 relates to amino acid modifications according to the “knob-into-hole” technology. In one embodiment of said CH3(KiH)-engineered antibody, in the CH3 domain of one heavy chain the amino acid T at position 366 (numbering according to EU index of Kabat) is substituted by W; and in the CH3 domain of the other heavy chain the amino acid Y at position 407 (numbering according to EU index of Kabat) is substituted by A. In another embodiment of said CH3(KiH)-engineered antibody, in the CH3 domain of one heavy chain the amino acid T at position 366 (numbering according to EU index of Kabat) is substituted by Y; and in the CH3 domain of the other heavy chain the amino acid Y at position 407 (numbering according to EU index of Kabat) is substituted by T.

In one embodiment within the antibody, which is of IgG2 isotype, comprised in the peptide-MHC-I-antibody fusion protein the approach described in WO 2011/090762 is used to support heterodimerization of the first heavy chain and the second heavy chain of the antibody.

In one embodiment within the antibody comprised in the peptide-MHC-I-antibody fusion protein the approach described in WO 2009/089004 is used to support heterodimerization of the first heavy chain and the second heavy chain of the antibody. In one embodiment of said CH3-engineered antibody, in the CH3 domain of one heavy chain the amino acid K or N at position 392 (numbering according to EU index of Kabat) is substituted by a negatively charged amino acid (in one preferred embodiment by E or D, in one preferred embodiment by D); and in the CH3 domain of the other heavy chain the amino acid D at position 399 the amino acid E or D at position 356 or the amino acid E at position 357 (numberings according to EU index of Kabat) is substituted by a positively charged amino acid (in one preferred embodiment K or R, in one preferred embodiment by K, in one preferred embodiment the amino acids at positions 399 or 356 are substituted by K). In one further embodiment, in addition to the aforementioned substitutions, in the CH3 domain of the one heavy chain the amino acid K or R at position 409 (numbering according to EU index of Kabat) is substituted by a negatively charged amino acid (in one preferred embodiment by E or D, in one preferred embodiment by D). In one even further embodiment, in addition to or alternatively to the aforementioned substitutions, in the CH3 domain of the one heavy chain the amino acid K at position 439 and/or the amino acid K at position 370 (numbering according to EU index of Kabat) is substituted independently from each other by a negatively charged amino acid (in one preferred embodiment by E or D, in one preferred embodiment by D).

In one embodiment within the antibody comprised in the peptide-MHC-I-antibody fusion protein the approach described in WO 2007/147901 is used to support heterodimerization of the first heavy chain and the second heavy chain of the antibody. In one embodiment of said CH3-engineered antibody, in the CH3 domain of one heavy chain the amino acid K at position 253 (numbering according to EU index of Kabat) is substituted by E, the amino acid D at position 282 (numbering according to EU index of Kabat) is substituted by K and the amino acid K at position 322 (numbering according to EU index of Kabat) is substituted by D; and in the CH3 domain of the other heavy chain the amino acid D at position 239 (numbering according to EU index of Kabat) is substituted by K, the amino acid E at position 240 (numbering according to EU index of Kabat) is substituted by K and the amino acid K at position 292 (numbering according to EU index of Kabat) is substituted by D.

In one embodiment within the antibody comprised in the peptide-MHC-I-antibody fusion protein the approach described in WO 2007/110205 is used to support heterodimerization of the first heavy chain and the second heavy chain of the antibody.

In one embodiment the peptide-MHC-I-antibody fusion protein comprises an antibody specifically binding to a target cell selected from the group of cancer cells or virus-infected cells. In one embodiment the target cell is a cancer cell. In one embodiment the target cell is a cancer cell of a non-solid tumor.

In one embodiment the cancer cell is selected from a cancer cell associated with one of the following types of cancer: lymphomas, lymphocytic leukemia, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers. In one embodiment the cancer cell is selected from a cancer cell associated with one of the following types of cancer: lymphomas, lymphocytic leukemia, bone cancer, and Hodgkin's Disease.

### Polypeptide of (ii):

In one embodiment, the polypeptide of (ii) of the peptide-MHC-I-antibody fusion protein is fused with its C-terminus to the N-terminus of one heavy chain of the antibody of (i).

In one embodiment, the polypeptide of (ii) of the peptide-MHC-I-antibody fusion protein comprises in N- to C-terminal direction a virus-derived peptide, a first peptide linker, a β2-microglobulin, a second peptide linker, and the extracellular domains α1, α2, and α3 of a class I MHC molecule.

In one embodiment, the polypeptide of (ii) of the peptide-MHC-I-antibody fusion protein is fused with its C-terminus to the N-terminus of one heavy chain of the antibody of (i) via a third peptide linker.

In one embodiment the peptide linker (first, second, or third peptide linker) comprises 8 to 50 amino acid residues. In one embodiment the peptide linker (first, second, or third peptide linker) comprises 8 to 25 amino acid residues.

In one embodiment the first and second peptide linker are identical in length. In one embodiment the first and second peptide linker are identical in their amino acid sequence.

In one embodiment the peptide linker is a glycine-serine linker. In one embodiment of the invention, the peptide linker is a peptide consisting of glycine and serine residues. In one embodiment of the invention, the glycine-serine linker is of the structure

(GxS)ₙ or (GxS)ₙGₘ

with G=glycine, S=serine, x=3 or 4, n=2, 3, 4, 5 or 6, and m=0, 1, 2 or 3.

In one embodiment, of above defined glycine-serine linkers, x=3, n=3, 4, 5 or 6, and m=0, 1, 2 or 3; or x=4, n=2, 3, 4 or 5 and m=0, 1, 2 or 3. In one preferred embodiment, x=4 and n=2 or 3, and m=0. In one preferred embodiment, x=4 and n=2. In one embodiment the peptide linker is (G₄S)₂.

In one embodiment, within the polypeptide of (ii) of the peptide-MHC-I-antibody fusion protein a disulfide bridge is formed between a cysteine residue of the first peptide linker and a cysteine residue of the extracellular domains α1, α2, and α3 of the class I MHC molecule.

In one embodiment the peptide-MHC-I-antibody fusion protein comprises exactly one polypeptide comprising in N- to C-terminal direction the virus-derived peptide, the β2-microglobulin and the extracellular domains α1, α2, and α3 of a class I MHC molecule.

In one embodiment the peptide-MHC-I-antibody fusion protein comprises one or two polypeptides comprising in N- to C-terminal direction the virus-derived peptide, the β2-microglobulin and the extracellular domains α1, α2, and α3 of a class I MHC molecule, wherein a disulfide bridge is formed between a cysteine residue of the first peptide linker and a cysteine residue of the extracellular domains α1, α2, and α3 of the class I MHC molecule.

In one embodiment the peptide-MHC-I-antibody fusion protein comprises two polypeptides comprising in N- to C-terminal direction the virus-derived peptide, the β2-microglobulin and the extracellular domains α1, α2, and α3 of a class I MHC molecule, wherein a disulfide bridge is formed between a cysteine residue of the first peptide linker and a cysteine residue of the extracellular domains α1, α2, and α3 of the class I MHC molecule.

In one embodiment the peptide-MHC-I-antibody fusion protein comprises a virus-derived a virus selected from human cytomegalovirus, adenovirus, human herpesvirus 1, human herpesvirus 2, human herpesvirus 4 (Epstein-Barr virus), hepatitis-B-virus, hepatitis-C-virus, human immunodeficiency virus, human papillomavirus type 16, human papillomavirus type 18, human papillomavirus type 31, human papillomavirus type 33, human papillomavirus type 35, human papillomavirus type 39, human papillomavirus type 45, human papillomavirus type 51, human papillomavirus type 52, human papillomavirus type 56, human papillomavirus type 58, human papillomavirus type 59, human papillomavirus type 68, human papillomavirus type 73, human papillomavirus type 82, human T-cell lymphotropic virus type I, human influenza A virus, human influenza B virus, vaccinia virus, dengue virus.

In one embodiment the virus-derived peptide is selected from NLVPMVATV (SEQ ID NO: 04), VLEETSVML (SEQ ID NO: 05), NLVPMVATV (SEQ ID NO: 06), RIFAELEGV (SEQ ID NO: 07), IIYTRNHEV (SEQ ID NO: 08), VLAELVKQI (SEQ ID NO: 09), AVGGAVASV (SEQ ID NO: 10), TVRSHCVSK (SEQ ID NO: 11), IMREFNSYK (SEQ ID NO: 12), GPISHGHVLK (SEQ ID NO: 13), ATVQGQNLK (SEQ ID NO: 14), VYALPLKML (SEQ ID NO: 15), AYAQKIFKIL (SEQ ID NO: 16), QYDPVAALF (SEQ ID NO: 17), YVKVYLESF (SEQ ID NO: 18), DIYRIFAEL (SEQ ID NO: 19), VFETSGGLVV (SEQ ID NO: 20), KARDHLAVL (SEQ ID NO: 21), QARLTVSGL (SEQ ID NO: 22), KARAKKDEL (SEQ ID NO: 23), QIKVRVDMV (SEQ ID NO: 24), RRRHRQDAL (SEQ ID NO: 25), ARVYEIKCR (SEQ ID NO: 26), KMQVIGDQY (SEQ ID NO: 27), NVRRSWEEL (SEQ ID NO: 28), CPSQEPMSIYVY (SEQ ID NO: 29), KPGKISHIMLDVA (SEQ ID NO: 30), ELRRKMMYM (SEQ ID NO: 31), IPSINVHHY (SEQ ID NO: 32), FEQPTETPP (SEQ ID NO: 33), YAYIYTTYL (SEQ ID NO: 34), QEFFWDANDIY (SEQ ID NO: 35), YEQHKITSY (SEQ ID NO: 36), QEPMSIYVY (SEQ ID NO: 37), SEHPTFTSQY (SEQ ID NO: 38), QAIRETVEL (SEQ ID NO: 39), TRATKMQVI (SEQ ID NO: 40), DALPGPCI (SEQ ID NO: 41), CEDVPSGKL (SEQ ID NO: 42), HERNGFTVL (SEQ ID NO: 43), PTFTSQYRIQGKL (SEQ ID NO: 44), QMWQARLTV (SEQ ID NO: 45), HELLVLVKKAQL (SEQ ID NO: 46), DDYSNTHSTRYV (SEQ ID NO: 47), SLYNTVATL (SEQ ID NO: 48), GLCTLVAML (SEQ ID NO: 49), GILGFVFTL (SEQ ID NO: 50), STNRQSGRQ (SEQ ID NO: 51), LLFGYPVYV (SEQ ID NO: 52), FAEGFVRAL (SEQ ID NO: 53), LIVIGILIL (SEQ ID NO: 54), or ILHTPGCV (SEQ ID NO: 55), WYAQIQPHW (SEQ ID NO: 56), AFSGVSWTM (SEQ ID NO: 57), ILIGVVITW (SEQ ID NO: 58), MMIPTVVAF (SEQ ID NO: 59), PFPQSNAPI (SEQ ID NO: 60), LLLTLLATV (SEQ ID NO: 61), IVLEHGSCV (SEQ ID NO: 62), LLFKTENGV (SEQ ID NO: 63), PLNEAIMAV (SEQ ID NO: 64), NLVRLQSGV (SEQ ID NO: 65), LVISGLFPV (SEQ ID NO: 66), LLLVAHYAI (SEQ ID NO: 67), LALLAAFKV (SEQ ID NO: 68), VILAGPMPV (SEQ ID NO: 69), HVLGRLITV (SEQ ID NO: 70), VTEHDTLLY (SEQ ID NO: 71), NTDFRVLEL (SEQ ID NO: 72), CVETMCNEY (SEQ ID NO: 73), or a variant thereof comprising of from 1 to 3 amino acid exchanges, additions, and/or deletions.

In one embodiment the virus-derived peptide is a human cytomegalovirus (huCMV) derived peptide. In one preferred embodiment the peptide has the amino acid sequence of SEQ ID NO: 4 NLVPMVATV. In one preferred embodiment the peptide has the amino acid sequence of SEQ ID NO: 74 SSPPMFRV.

In one embodiment the virus-derived peptide consists of 5 to 15 amino acids. In one embodiment the virus-derived peptide consists of 7 to 12 amino acids. In one embodiment the virus-derived peptide consists of 7 to 10 amino acids.

In one embodiment the virus derived peptide comprised in peptide-MHC-I-antibody fusion protein is identical to the virus-derived peptide comprised on the antibody-peptide fusion protein. In one embodiment the virus derived peptide comprised in peptide-MHC-I-antibody fusion protein and the virus-derived peptide comprised on the antibody-peptide fusion protein consist of the identical amino acid sequence.

In one embodiment the peptide-MHC-I-antibody fusion protein comprises a class I MHC molecule selected from the group of HLA-A*0101, HLA-A*0201, HLA-A*0301, HLA-B*0702, HLA-B*0801, HLA-B*4402, HLA-C*0401, HLA-C*0501, HLA-C*0701, HLA-C*0702, HLA-A*0201, HLA-A*1101, HLA-A*2402, HLA-A*340101, HLA-B*1301, HLA-B*1521, HLA-B*5601, HLA B*5602, HLA-C*0102, HLA-C*0401, HLA-C*0403, HLA C*1502, HLA-A*0201, HLA-A*2402, HLA C*0202, HLA-C*0304, HLA-C*0401, HLA-C*0702, HLA-A*1101, HLA-A*2402, HLA B*1504, HLA-C*0102, HLA-C*0304, HLA-C*0702, and HLA C*0801.

In one embodiment the peptide-MHC-I-antibody fusion protein comprises a class I MHC molecule selected from the group of HLA-A*0201, or HLA-A*1101, or HLA-A*2402, or HLA-A*340101, or HLA C*0304, or HLA-C*0401, or HLA-C*070.

In one embodiment the peptide-MHC-I-antibody fusion protein comprises a class I MHC molecule selected from the group of HLA-A*0101, HLA-A*0201, HLA-A*0301, HLA-B*0702, HLA-B*0801, HLA-B*4402, HLA-C*0401, HLA-C*0501, HLA-C*0701, and HLA-C*0702.

In one embodiment the peptide-MHC-I-antibody fusion protein comprises a class I MHC molecule selected from the group of HLA-A*0201, HLA-A*1101, HLA-A*2402, HLA-A*340101, HLA-B*1301, HLA-B*1521, HLA-B*5601, HLA B*5602, HLA-C*0102, HLA-C*0401, HLA-C*0403, and HLA C*1502.

In one embodiment the peptide-MHC-I-antibody fusion protein comprises a class I MHC molecule selected from the group of HLA-A*0201, HLA-A*2402, HLA C*0202, HLA-C*0304, HLA-C*0401, and HLA-C*0702.

In one embodiment the peptide-MHC-I-antibody fusion protein comprises a class I MHC molecule selected from the group of HLA-A*1101, HLA-A*2402, HLA B*1504, HLA-C*0102, HLA-C*0304, HLA-C*0702, and HLA C*0801.

### Antibody-Peptide Fusion Protein

The following paragraphs include embodiments further defining the peptide-MHC-I-antibody fusion protein.

### Anti-XCR1 Antibody

In one embodiment the antibody-peptide fusion protein comprises an antibody specifically binding to human XCR-1.

A suitable monoclonal anti-XCR1 antibody is for example mAb 6F8 disclosed in WO 2009/065561 or selected from humanized anti-human-XCR1 antibodies HK1L2 and HK5L5 disclosed in WO 2013/032032.

Antibodies specificantly binding to XCR1 can be obtained by direct injection of XCR1 or a fragment thereof into an animal or by administering XCR1 or a fragment thereof to an animal, preferably a non-human. The antibody so obtained will then bind to XCR1. For preparation of monoclonal antibodies, any technique known in the art, which provides antibodies produced by continuous cell line cultures, e.g. a hybridoma cell line, can be used. The production of a suitable monoclonal antibody is also detailed in Example 7 of WO 2009/065561, which is incorporated by reference herein. Techniques described for the production of single chain antibodies (U.S. Pat. No. 4,946,778) can be adapted to produce single chain antibodies to XCR1. Also, transgenic mice or other organisms such as other mammals may be used to express humanized antibodies to XCR1.

Antibodies specificantly binding to XCR1 may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, N.J., 2001) and in Marks and Bradbury in Methods in Molecular Biology 248:161-175 (Lo, ed., Human Press, Totowa, N.J., 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. in EMBO Journal 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in the Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antibodies or antibody fragments isolated from human antibody libraries are considered human antibodies or human antibody fragments herein.

According to the invention, the antibody specifically binding to XCR1 is covalently coupled to a virus-derived peptide. Embodiments relating to the virus-derived peptide are presented above in the chapter dealing with the peptide-MHC-I-antibody fusion protein. Those embodiments apply to the virus-derived peptide that is coupled to the anti-XCR1 antibody as well. In one embodiment the virus-derived peptide present in the peptide-MHC-I-antibody fusion protein and the virus-derived peptide present in the antibody-peptide fusion protein are derived from the same virus. In one embodiment the virus-derived peptide present in the peptide-MHC-I-antibody fusion protein and the virus-derived peptide present in the antibody-peptide fusion protein are identical. In one embodiment the virus-derived peptide present in the antibody-peptide fusion has the identical amino acid sequence as the virus-derived peptide bound to the class I MHC expressing, antigen presenting cell.

### Class I MHC Expressing, Antigen Presenting Cell

The “class I MHC expressing, antigen presenting cell” according to the invention may be any suitable class I MHC expressing, antigen presenting cell. In one embodiment the peptide-loaded major histocompatibility complex class I (MHC-I) presenting cell is a blood cell, in one embodiment a peripheral blood mononucleated cell (PBMC), in one embodiment a monocyte or a lymphocytic cell. In one embodiment the peptide-loaded major histocompatibility complex class I (MHC-I) presenting cell is a monocyte or a lymphocytic cell of the patient. In one embodiment the peptide-loaded major histocompatibility complex class I (MHC-I) presenting cell is a monocyte or a lymphocytic cell that is isolated from the patient.

The amplification step pursuant to the embodiments of the present invention includes the administration of peptide-loaded major histocompatibility complex class I (MHC-I) presenting cell to the patient.

Various methods are available to obtain a peptide-loaded major histocompatibility complex class I (MHC-I) presenting cell of step i) of the embodiments of the invention.

In one preferred embodiment, this can be done in vitro by external loading with a peptide, such as SIINFEKL (SEQ ID NO:86) in the Examples as model epitope-comprising peptide in the protein OVA, comprising the antigen of interest. Typically, the cells are incubated with the peptides in a suitable fluid such an aqueous solution or medium for a certain time period, such as for 10 minutes to 24 hours or 48 hours, in particular for 20 minutes to 12 hours.

As peptides presented in the context of MHC-I typically have a length 8, 9, 10 or 11 amino acids, i.e. this length is required for binding of the peptides in the context of MHC-I, the peptides used for external loading preferably have this length.

Therefore, in a further preferred embodiment of the medicament for use or method of the invention, the peptide-loaded major histocompatibility complex class I (MHC-I) presenting cell of step i) in the embodiments of the present invention is obtained by incubating at least one major histocompatibility complex class I (MHC-I) presenting cell with said peptide(s) in vitro.

In case of externally loading cells in vitro, the peptide sequence may be chosen by methods known in the art. Externally loading cells in vitro can be performed by methods known in the art, e.g. by providing an aqueous solution of the peptides, adding the solution to the cells, which are preferably in a buffered solution or medium, incubating the cells with the peptides as to achieve a high saturation of the MHC-I with the respective peptide, and optionally washing the cells, e.g. with an aqueous solution.

Said culturing is performed as described below in more detail in order to achieve the loading with peptides derived from the antigen-comprising protein. As described below, both external loading and internal loading methods are possible. For example, external loading may be possible by culturing in the presence of externally added peptide(s). In case of internal loading procedures, such as by using cell-penetrating peptides fused to the antigen-comprising protein, more time may be preferably required to allow the cells to process the protein and present the resulting peptides in the context of MHC-I.

External loading of the MHC-I with peptides poses certain limitations in the human due to the heterogeneity of MHC-I molecules in the human population. As a result, different peptides derived from the same antigen (e.g. nucleoprotein of influenza of TRP-1 from melanoma) are preferably to be used in different individuals for amplification. This problem can be overcome with an “internal” loading of the MHC-I bearing cells used which whole protein or a long peptide containing many antigenic epitopes. To this end, the unprocessed antigen-comprising protein or a fragment thereof comprising the antigen(s) or epitope(s) is introduced into the MHC-I bearing cells, whereupon it is enzymatically broken down (“processed”) into a plurality of different peptides, which are then presented on the surface in the context of MHC-I (and MHC-II).

The transport of the unprocessed antigen-comprising protein or a fragment thereof comprising the antigen into the cell can be achieved with a variety of physical or chemical methods which are known to a skilled person, such as, but not limited to, electroporation, forced endocytosis, injection, and cell-penetrating peptides.

Another way to internally load MHC-I bearing cells is to load them with nucleic acids, such as DNA or RNA, coding for the antigen-comprising protein or a fragment thereof comprising the antigen. The introduction of nucleic acid into the cells can be by any chemical, physical, or biological means, such as, but not limited to electroporation, injection, transfection, or infection with organisms recombinantly modified to carry the nucleic acid sequence coding for the antigen-comprising protein or a fragment thereof comprising the antigen. Once the coded protein is expressed by the cell machinery, it is processed and presented on the cell surface in the context of MHC-I (and MHC-II) and the cell becomes a peptide-loaded cell in accordance with the present invention. The skilled person is aware of suitable DNA and RNA constructs and DNA- or RNA-based expression systems. For example, suitable RNA constructs or RNA-based expression systems preferably further comprise elements which allow for translation and therefore expression of a protein in the cell of interest. For example, suitable DNA constructs or DNA-based expression systems preferably further comprise elements which allow for transcription and translation and therefore expression of a protein in the cell of interest. Optionally, such systems further comprise suitable elements which allow for replication of the DNA or RNA construct, or DNA- or RNA-based expression system, respectively.

For example viral systems, and non-viral expression systems, which are capable of expressing a protein of interest (in this case the antigen-comprising protein or a fragment thereof comprising the antigen) in the major histocompatibility complex class I (MHC-I) presenting cell, are suitable for this purpose.

Therefore, in a further preferred embodiment of the medicament for use or method of the invention, the peptide-loaded major histocompatibility complex class I (MHC-I) presenting cell of step i) is obtained by:

applying a physical, chemical or biological method for introducing an antigen-comprising protein or a fragment thereof comprising the antigen, or a nucleic acid encoding the antigen-comprising protein or a fragment thereof comprising the antigen into at least one major histocompatibility complex class I (MHC-I) presenting cell,

allowing the at least one cell to express the antigen-comprising protein or a fragment thereof comprising the antigen into at least one major histocompatibility complex class I (MHC-I) presenting cell in case of a nucleic acid, and

allowing the at least one cell to process the antigen and present the peptides in the context of MHC-I.

In particular, following embodiments are preferred for obtaining such cell:
a. incubating at least one major histocompatibility complex class I (MHC-I) presenting cell with the antigen-comprising protein or a fragment thereof comprising the antigen, and allowing the at least one cell to process the antigen and present the peptides in the context of MHC-I, or
b. exposing at least one major histocompatibility complex class I (MHC-I) presenting cell to a viral system which is capable of (i) infecting said cell and (ii) expressing the antigen-comprising protein or a fragment thereof comprising the antigen in the cell, and allowing the at least one cell to process the antigen-comprising protein antigen or fragment thereof comprising the antigen, and allowing the at least one cell to present the peptide(s) in the context of MHC-I, or
c. transfecting at least one major histocompatibility complex class I (MHC-I) presenting cell with DNA, or RNA, or a DNA- or RNA-based expression system which is comprising a nucleic acid coding for the antigen-comprising protein or a fragment thereof comprising the antigen, and
   allowing the at least one cell to express and process the antigen-comprising protein antigen or fragment thereof comprising the antigen, and allowing the at least one cell to present the peptides in the context of MHC-I, or
d. incubating at least one major histocompatibility complex class I (MHC-I) presenting cell with a compound comprising a cell-penetrating peptide (CPP) and the antigen-comprising protein or a fragment thereof comprising the antigen, and
   allowing the at least one cell to process said compound, and
   allowing the at least one cell to present the peptides in the context of MHC-I.

A compound comprising a cell-penetrating peptide (CPP) and the antigen-comprising protein or a fragment thereof comprising the antigen is understood as a compound wherein a cell-penetrating peptide (CPP) is chemically linked the antigen-comprising protein or a fragment thereof comprising the antigen, optionally by a suitable linker. Such linker may for example be a peptide linker, for example a peptide linker having a length of 1 to 50, preferably 1 to 20, more preferably 1, 2, 3, 4, 5, 6, 7, 8, 9, or 10 amino acids, however, also other linkers can be used. In one preferred embodiment, the compound is a fusion protein comprising a cell-penetrating peptide (CPP) and the antigen-comprising protein or a fragment thereof comprising the antigen, which fusion protein may optionally contain a suitable peptide linker. In one embodiment, the compound is a fusion protein consisting of a cell-penetrating peptide (CPP) and the antigen-comprising protein or a fragment thereof comprising the antigen, wherein the cell-penetrating peptide (CPP) and the antigen-comprising protein or a fragment thereof comprising the antigen are linked via a peptide bond. In a preferred embodiment, the cell-penetrating peptide (CPP) is located N-terminally or C-terminally to the antigen-comprising protein or a fragment thereof comprising the antigen in the compound.

It is preferred that the peptide-loaded major histocompatibility complex class I (MHC I) presenting cell is not cell derived from a cell line, or a cell obtained from a mammal which was cultivated in vitro for a longer time period, such as more than 3 weeks, as a longer in vitro cultivation of cells results in phenotypic changes of such cells. Accordingly, it is preferred to use primary cells, i.e. cells which are obtained from mammal and which are either not cultivated in vitro or are cultivated in vitro for up to 3 weeks, 20 days, 14 days, 10 days, 9 days, 8 days, 7 days, 6 days, 5 days, 4 days or 3 days.

Therefore, in another preferred embodiment of the present invention, the peptide-loaded major histocompatibility complex class I (MHC I) presenting cell is a primary cell.

Instead of using primary cells such as PBMC, T cells, B cells, or monocytes, as described herein, for amplification, it is also possible to use primary cells expanded to high cell numbers in vitro for amplification. These expanded cells would be either externally loaded with peptides, or internally with proteins; alternatively they would be exposed to vectors coding for peptides or proteins, as described in detail herein. For example, it is possible to start with a small PBMC sample and expand either T cells or B cells from the sample by in vitro culture for up to 3 weeks, such as up to 14 days. This procedure is possible with or without prior separation of T cells or B cells. The in vitro expansion process is technically feasible using GMP-compatible closed systems and employing T cell- or B-cell activating systems. For T cells, the T cell receptor-complex is triggered with suitable agents, e.g. antibodies, and expansion of the T cells is ensured by adding (a) growth factor(s), in particular a mixture of growth factors, typically including IL-2. For B cells, the B-cell receptor-complex is triggered with suitable agents, followed by addition of appropriate growth factor(s). After expansion of T cells or B cells to high cell numbers, such as 1×10⁹ to 10×10¹⁰, the cells are externally loaded with peptide(s) or internally loaded with long peptides or proteins, or expression systems for long peptides or proteins, for a short period of time, such as up to 2 days, as described in detail herein and used for the amplification procedure together with an Th1 adjuvant which is a danger signal.

In another preferred embodiment of the present invention, the peptide-loaded major histocompatibility complex class I (MHC I) presenting cell is a cell selected from a monocyte, a T cell and a B cell.

For example, monocytes, T cells, B cells or mixtures thereof may be loaded with peptide. PBMC samples consist of T cells, B cells, NK cells, and monocytes. There is a tiny population 0.5 to 1% of DC in peripheral blood and they are not regarded as PBMC cells. When PBMC samples are used as a sample for amplification, the few contaminating DC are functionally irrelevant compared to the vast majority 99-99.5% of MHC-I-presenting cells in a PBMC sample. Thus in terms of function for amplification, a sample of PBMC is regarded as a mixture of T cells, B cells, NK cells, and monocytes only.

For example, monocytes, T cells, B cells may be loaded by using a PBMC sample directly, or the cell types or mixtures thereof may be loaded after purification from a suitable source, such as a PBMC sample.

Accordingly, in one preferred embodiment, the cell is a PBMC cell or a mixture of different PBMC cells.

The peptide-loaded major histocompatibility complex class I (MHC I) cell may be an allogeneic cell or an autologous cell. In another preferred embodiment of the present invention, the peptide-loaded major histocompatibility complex class I (MHC I) presenting cell is an autologous cell.

In another preferred embodiment of the present invention, the peptide-loaded major histocompatibility complex class I (MHC I) presenting cell is not a tumor cell or hyperproliferative cell. Such cells are preferably not used in order to avoid any unwanted hyperproliferation in the patient's body.

As explained above, it is preferred not use dendritic cells peptide-loaded major histocompatibility complex class I (MHC I) presenting cell. Therefore, in another preferred embodiment of the present invention, the peptide-loaded major histocompatibility complex class I (MHC I) presenting cell is not a dendritic cell (DC).

In another preferred embodiment of the present invention, the peptide-loaded major histocompatibility complex class I (MHC I) presenting cell is administered systemically.

In another preferred embodiment of the present invention, the peptide-loaded major histocompatibility complex class I (MHC I) presenting cell is administered intravenously, such as by intravenous injection or infusion.

In another preferred embodiment of the present invention, the peptide-loaded major histocompatibility complex class I (MHC I) presenting cell is administered at a dose of between 1×10⁸ and 100×10⁹ peptide-loaded major histocompatibility complex class I (MHC-I) presenting cells. More preferred dose ranges are a dose of between 5×10⁸ and 50×10⁹, 5×10⁸ and 20×10⁹, 5×10⁸ and 10×10⁹, 5×10⁸ and 5×10⁹, 5×10⁸ and 20×10⁸, between 5×10⁸ and 10×10⁸, between 1×10⁹ and 100×10⁹, 1×10⁹ and 50×10⁹, 1×10⁹ and 20×10⁹, 1×10⁹ and 10×10⁹, 1×10⁹ and 5×10⁹, 1×10⁹ and 2×10⁹ and between 1×10⁸ and 2×10⁹ peptide-loaded major histocompatibility complex class I (MHC-I) presenting cells.

As described above, it is possible to use a population of peptide-loaded major histocompatibility complex class I (MHC I) presenting cells comprising 2 or more different cell types. In a preferred embodiment, such population is a PBMC sample, which comprises the cell types T cells, B cells and monocytes.

Therefore, in another preferred embodiment of the present invention, a population of peptide-loaded major histocompatibility complex class I (MHC I) presenting cells is administered in step i).

In a more preferred embodiment of the invention, the population of peptide-loaded major histocompatibility complex class I (MHC I) presenting cells is a PBMC sample.

In another more preferred embodiment of the present invention, the population of peptide-loaded major histocompatibility complex class I (MHC I) presenting cells comprises T cells and/or B cells.

The embodiments of the present invention make use of Th1 adjuvants which are danger signals. In case an embodiment of the present invention makes use of more than one Th1 adjuvant which is a danger signal, such Th1 adjuvants which are a danger signal are selected independently from each other and may be the same or different.

Therefore, in a one embodiment, the class I MHC (major histocompatibility complex) expressing, antigen presenting cell class I is obtained from the patient and is cultured between 2 h and 3 days, more preferably between 2 h and 2 days in vitro.

In one embodiment the class I MHC expressing, antigen presenting cell is a primary PBMC, which is optionally cultivated in vitro for up to 3 days. In one embodiment, the class I MHC expressing, antigen presenting cell is a cell selected from a monocyte, a T cell and a B cell. In one embodiment the class I MHC expressing, antigen presenting cell is a T cell or a B cell. For example, monocytes, T cells, B cells or mixtures thereof may be loaded with peptide. PBMC samples consist of T cells, B cells, NK cells, and monocytes. There is a tiny population 0.5 to 1% of dendritic cells (DCs) in peripheral blood and they are not regarded as PBMC cells. When PBMC samples are used, the few contaminating DC are functionally irrelevant compared to the vast majority 99-99.5% of MHC-I-presenting cells in a PBMC sample. Thus in terms of function for amplifying, a sample of PBMC is regarded as a mixture of T cells, B cells, NK cells, and monocytes only. In one embodiment the “class I MHC expressing, antigen presenting cell” as used herein is not a dendritic cell. For example, monocytes, T cells, B cells may be loaded by using a PBMC sample directly, or the cell types or mixtures thereof may be loaded after purification from a suitable source, such as a PBMC sample. Accordingly, in one preferred embodiment, the cell is a PBMC cell or a mixture of different PBMC cells. The class I MHC expressing, antigen presenting cell may be an allogeneic cell or an autologous cell. In another preferred embodiment of the present invention, the class I MHC expressing, antigen presenting cell is an autologous cell. The “class I MHC expressing, antigen presenting cell” as used herein is is not a tumor cell or pathogenic hyperproliferative cell.

Peptide-loaded PBMCs (peripheral blood mononuclear cells) are preferred cells, which allow for an easy way for amplification. Also other autologous cells may be applicable, such as in vitro expanded B cells or T cells. This can be done e.g. in a rather automated fashion in closed systems. For particle reasons and regulatory purposes, cells not suitable include dendritic cells, in vitro generated dendritic cells, tumor tissues removed from a patient, or tumor cell lines.

The class I MHC expressing, antigen presenting cell may be administered systemically, such as intravenously, e.g. by intravenous injection or infusion.

In another preferred embodiment of the present invention, the peptide-loaded major histocompatibility complex class I (MHC I) presenting cell is administered at a dose of between 1×10⁸ and 100×10⁹ peptide-loaded major histocompatibility complex class I (MHC-I) presenting cells. More preferred dose ranges are a dose of between 5×10⁸ and 50×10⁹, 5×10⁸ and 20×10⁹, 5×10⁸ and 10×10⁹, 5×10⁸ and 5×10⁹, 5×10⁸ and 20×10⁸, between 5×10⁸ and 10×10⁸, between 1×10⁹ and 100×10⁹, 1×10⁹ and 50×10⁹, 1×10⁹ and 20×10⁹, 1×10⁹ and 10×10⁹, 1×10⁹ and 5×10⁹, 1×10⁹ and 2×10⁹ and between 1×10⁸ and 2×10⁹ peptide-loaded major histocompatibility complex class I (MHC-I) presenting cells.

In case of inducing a cellular cytotoxic immune response towards the virus-derived peptide by treatment, the amplifying medicament is preferably administered to the patient within 5 to 9 days after the inducing medicament. The amplification can be repeated, e.g. several times, with several weeks inbetween in order to increase efficiency of the amplification.

### Expression of Fusion Proteins

The fusion proteins used according to the invention may be produced by recombinant methods known in the art. Antibodies may be produced using recombinant methods and compositions, e.g., as described in U.S. Pat. No. 4,816,567. Peptide-MHC-I-antibody fusion protein may be produced using the recombinant methods as described in WO 2012/175508, WO 2014/083004 and WO 2014/096015.

### Pharmaceutical Formulations

Pharmaceutical formulations of the fusion proteins used according to the invention as described herein are prepared by mixing such fusion proteins having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration are generally sterile. Sterility may be readily accomplished, e.g., by filtration through sterile filtration membranes.

### Therapeutic Methods and Compositions

Any of the peptide-MHC-I antibody fusion proteins provided herein may be used in therapeutic methods and/or as therapeutic medicament, i.e. in order to treat disease.

A fusion protein as provided herein (and any further medicament or agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Fusion proteins as provided herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The fusion protein need not be, but is optionally formulated with one or more agents currently used to prevent or treat the disorder in question. The effective amount of such other agents depends on the amount of fusion protein present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a fusion protein of the invention (when used alone or in combination with one or more other additional therapeutic agents) will depend on the type of disease to be treated, the type of fusion protein, the severity and course of the disease, whether the fusion protein is administered for preventive or therapeutic purposes, previous therapy, the patient's clinical history and response to the fusion protein, and the discretion of the attending physician. The fusion protein is suitably administered to the patient at one time or over a series of treatments. Depending on the type and severity of the disease, about 1 μg/kg to 15 mg/kg (e.g. 0.1 mg/kg-10 mg/kg) of fusion protein can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion protein would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion protein). An initial higher loading dose, followed by one or more lower doses may be administered.

Compositions of the invention may be a kit of parts, which relates to he juxtaposition of separate but functionally interacting individual components. The kit of parts may be composed of at least two of the following medicaments as described herein: The therapeutic medicament, the amplifying medicament and the inducing medicament. Providing these components forms a functional unity, as described above.

The peptide-MHC-I-antibody fusion protein or kits of parts may be used in a single therapeutic administration, i.e. each medicament only once. However, the therapy may also be administered in cycles, each cycle comprising administration of amplifying medicament and therapeutic medicament. There may be a rest between the circles to allow for recreation of the patients. Typical durations of rests are one month, 6 week, two months or three months. Typically, the disease to be treated is monitored during or between the circles.

### Inducing and Amplifying the Cellular Cytotoxic Immune Response

In one embodiment a cellular cytotoxic immune response towards the virus-derived peptide is amplified in the patient by an amplifying medicament. The amplifying medicament may comprise a class I MHC expressing, antigen presenting cell, wherein the MHC-I is bound to the virus-derived peptide, and a Th1 adjuvant, which is a danger signal.

The administration of the cells may be performed by a method known to a skilled person, in particular using various administration routes. In a preferred embodiment, the cells are living cells. Typically, the cells are prepared as a suspension of cells in an aqueous solution, such as a physiologically acceptable aqueous solution, which is preferably buffered. For example, such solution is a physiologically acceptable buffered solution with physiological saline concentration. The peptide-loaded class I MHC expressing, antigen presenting cell is administered systemically. In one embodiment the peptide-loaded class I MHC expressing, antigen presenting cell is administered intravenously, intraperitoneally, or by intrathecal injection or alternatively, subcutaneously, or by administration into the tumor in case the patient suffers from a tumor. In one embodiment, the peptide-loaded class I MHC expressing, antigen presenting cell is administered intravenously.

The amount of the cells to be administered may vary depending on various factors such as the antigen and patient. For example, the upper amount of cells to be administered may be limited by the amount of cells which can be obtained initially from the patient for generating the class I MHC expressing, antigen presenting cells. Typically, between 1×10⁶ and 100×10⁹ cells may be administered to humans. For, example, a dose of between 1×10⁸ and 100×10⁹ peptide-loaded major histocompatibility complex class I (MHC-I) presenting cells. More preferred dose ranges are a dose of between 5×10⁸ and 50×10⁹, 5×10⁸ and 20×10⁹, 5×10⁸ and 10×10⁹, 5×10⁸ and 5×10⁹, 5×10⁸ and 20×10⁸, between 5×10⁸ and 10×10⁸, between 1×10⁹ and 100×10⁹, 1×10⁹ and 50×10⁹, 1×10⁹ and 20×10⁹, 1×10⁹ and 10×10⁹, 1×10⁹ and 5×10⁹, 1×10⁹ and 2×10⁹ and between 1×10⁸ and 2×10⁹ peptide-loaded major histocompatibility complex class I (MHC-I) presenting cells may be administered to a human patient. However, as described above, it is preferred that higher amount of class I MHC expressing, antigen presenting cells are administered.

In one embodiment administration of the class I MHC expressing, antigen presenting cell is performed systemically, in particular intravenously, intraperitoneally, or by intrathecal injection, and that between 1×10⁶ and 100×10⁹ class I MHC expressing, antigen presenting cells are administered to said patient. Therefore, in a yet further preferred embodiment of the medicament for use or method of the invention, between 1×10⁷ and 4×10⁸ class I MHC expressing, antigen presenting cells are administered to said patient, more preferably a dose of between 5×10⁸ and 50×10⁹, 5×10⁸ and 20×10⁹, 5×10⁸ and 10×10⁹, 5×10⁸ and 5×10⁹, 5×10⁸ and 20×10⁸, between 5×10⁸ and 10×10⁸, between 1×10⁹ and 100×10⁹, 1×10⁹ and 50×10⁹, 1×10⁹ and 20×10⁹, 1×10⁹ and 10×10⁹, 1×10⁹ and 5×10⁹, 1×10⁹ and 2×10⁹ and between 1×10⁸ and 2×10⁹ peptide-loaded major histocompatibility complex class I (MHC-I) presenting cells As described above and in the examples, the administration of class I MHC expressing, antigen presenting cells to the patient in a time frame of from 3 days to 14 days, preferably 4 days to 10 days, results in amplification and/or extension of the cellular cytotoxic response. Preferably, the cells are administered only once within the time frame.

In one embodiment the of class I MHC expressing, antigen presenting cells is administered in a time frame of from 3 days to 14 days, preferably 4 days to 10 days, after administration of the inducing medicament (e.g. antibody-peptide fusion protein or the XCL1-peptide fusion protein). In one embodiment the of class I MHC expressing, antigen presenting cells is administered in a time frame of from 5 to 9 days after administration of the inducing medicament (e.g. antibody-peptide fusion protein or the XCL1-peptide fusion protein).

The adjuvant is an adjuvant that supports a Th1-mediated immune response. In one embodiment the adjuvant that supports a Th1-mediated immune response is poly I:C (polyinosinic:polycytidylic acid).

In one embodiment, interleukin-2 (IL-2) or IL-2 variant with extended half-life in the patient has been or is administered after the administration of the class I MHC expressing, antigen presenting cell. In one embodiment, administration of IL-2 is performed after administration of the class I MHC expressing, antigen presenting cells. In one embodiment IL-2 (in one embodiment IL-2cx) is administered in a time frame of from 0 h to 10 days, preferably 0 h to 5 days, after the administration of the class I MHC expressing, antigen presenting cells, preferably within 24 hours from the administration of the class I MHC expressing, antigen presenting cell. In one embodiment, the IL-2 component has to be applied continuously (e.g. every 8 h or truly continuously). Maximum duration of IL-2 application is estimated to be around 1 week (possibly up to 2 weeks).

In a preferred embodiment of the present invention, a hIL-2 variant with extended circulating half-life in vivo in the patient as compared to hIL-2 is/has been administered, wherein the hIL-2 variant is capable of binding to the high affinity IL-2 receptor chain (CD25). It was surprisingly found that a strong amplification of the cytotoxic immune response is achieved, when, administering to the patient a hIL-2 variant with extended circulating half-life in vivo in the patient as compared to hIL-2, wherein the hIL-2 variant is capable of binding to the high affinity IL-2 receptor chain (CD25), wherein the administration of the hIL-2 variant of step ii) begins within 5 days after the administration of the peptide-loaded major histocompatibility complex class I (MHC I) presenting cell, and wherein the hIL-2 variant of step ii) is administered at least once daily or continuously, and is administered for at least 2 subsequent days.

In a preferred embodiment of the present invention, the hIL-2 variant has a circulating half-life in vivo of at least 2 hours in a human and/or comprises at least one hIL-2 moiety.

Accordingly, the hIL-2 variant may comprise 1, 2, 3, 4, 5 or more hIL-2 moieties.

An “hIL-2 variant with extended circulating half-life in vivo in a human as compared to hIL-2”, is also designated “IL-2 with extended half-life”, or “IL-2ext” in the present application. Cytokines are molecules which are known to have a short half-life due to glomerular filtration in the kidney. hIL-2 reportedly has a half-life of of several minutes in vivo in the human. It has been previously determined that the half-life of a cytokine can be substantially extended in vivo by attaching it to larger, functionally inert molecule which serves as a “scaffold”. This scaffold increases the hydrodynamic volume of the attached cytokine, thus preventing its rapid excretion from a body. Various scaffolds of this kind are known to the person skilled in the art. “hIL-2 variant with extended circulating half-life in vivo in a human as compared to hIL-2” or “IL-2ext” is therefore understood as a molecule comprising a human IL-2 (hIL-2), which is non-covalently or covalently, preferably covalently bound to a scaffold, and shows a half-life in a human patient in vivo of at least 2 hours. “IL-2ext” of the present invention is capable of binding to the high affinity IL-2 receptor chain CD25. In a preferred embodiment, “IL-2ext” binds to both the high-affinity (CD25) IL-2 receptor and the low-affinity (CD122) IL-2 receptor. In one preferred embodiment, the human IL-2 (hIL-2) is covalently bound to the scaffold recombinantly or chemically.

Suitable popular scaffolds for use in the invention are known in the art, and comprise immunoglobulins, comprising complete antibodies, Fc-regions of antibodies, and other antibody formats such as antibodies without Fc-region (Diabodies), single chain Fv (scFv) fusions, Fab-scFv fusions and other antibody-derived formats increasing the half-life of the cytokine. Such antibodies merely serve to increase half-life in vivo. Accordingly, the antibody portion preferably does not specifically recognize an epitope of the patient's body. Accordingly, in a preferred embodiment, such antibodies for use as scaffold do not specifically recognize an epitope of the patient. Therefore, in one embodiment, an “antibody” is understood as protein comprising at least one Ig-like domain, preferably comprising at least one antigen-binding domain. In a preferred embodiment, at least one antigen-binding domain comprises a VH and/or a VL domain. In case an antibody is used, the antibody is preferably not capable of specifically binding to or recognizing a human antigen. Such antibodies are known in the art and used in the Examples.

Other preferred scaffolds are large human proteins which are functionally inert, such as human serum albumin. Further preferred scaffolds to which IL-2 can be bound are polymers such as PEG or polymer mimetics such as hydrophilic and flexible polypeptide chains as used in the XTEN or PASylation technologies. Further preferred scaffolds increasing the half-life of IL-2 are carbohydrates, such as dextran, polysialic acids, hyaluronic acid, dextrin, or hydroxyethyl starch.

Accordingly, the IL-2 variant used in the present invention preferably exhibits an increased molecular weight as compared to IL-2.

Therefore, in another preferred embodiment of the present invention, the IL-2 variant has a molecular weight of at least 30 kDa and/or wherein the IL-2 moiety in the IL-2 variant is covalently linked to a chemical moiety having a molecular weight of at least 15 kDa.

Preferably, the IL-2 variant has a molecular weight of at least 40 kDa, 50 kDa, 80 kDa, 100 kDa, or 200 KDa. For example, the molecular weight may be up to about 3000 kDa, 2000 kDa or 1000 kDa.

Preferably, the IL-2 moiety in the IL-2 variant is covalently linked to a chemical moiety having a molecular weight of at least 25 kDa, 35 kDa, 65 kDa, 85 kDa, or 185 kDa. For example, the molecular weight of the chemical moiety may be up to about 3000 kDa, 2000 kDa or 1000 kDa.

In another preferred embodiment of the present invention, the IL-2 variant is selected from a PEGylated IL-2, IL-2 linked to hyaluronic acid and IL-2 fused to at least one peptide or protein, preferably wherein the IL-2 fused to a least one peptide or protein is IL-2 fused to an Fc, IL-2 fused to XTEN, IL-2 fused to an immunoglobulin, preferably antibody, which is not capable of specific binding to a human antigen, IL-2 fused to Transferrin, IL-2 fused to Albumin, preferably human serum Albumin, IL-2 fused to PEG, IL-2 fused to a homoamino acid polymer (HAP), IL-2 fused to a proline-alanine-serine (PAS) polymer, IL-2 fused to a carbohydrate, more preferably selected from dextran, polysialic acids, hyaluronic acid, dextrin, and hydroxyethyl starch (HES), or IL-2 fused to a elastin-like peptide (ELP). Accordingly, in one preferred embodiment, the “scaffold” is selected from a PEG moiety, such as PEG50, PEG1000 or PEG2000, XTEN, hyaluronic acid, at least one peptide or protein, an immunoglobulin which is not capable of specific binding to a human antigen, an Fc, Transferrin, Albumin, recombinant PEG, a homoamino acid polymer (HAP), a proline-alanine-serine (PAS) polymer, carbohydrate, more preferably selected from dextran, polysialic acids, hyaluronic acid, dextrin, and hydroxyethyl starch (HES), and an elastin-like peptide (ELP).

In case of covalent linkage to a scaffold which is a protein, the IL-2 may be linked to the N-terminus, the C-terminus or internally, via an amino acid side chain of the scaffold. In case of covalent linkage to a scaffold which is antibody, the IL-2 may be linked to the C-terminus of the light chain or fragment thereof, to the C-terminus of the heavy chain or fragment thereof, to the N-terminus of the light chain or fragment thereof or to the N-terminus of the heavy chain or fragment thereof, or internally, via a amino acid side chain of heavy and/or light chain. In case of covalent linkage to a scaffold which is an Fc molecule, the IL-2 may be linked to the N-terminus, the C-terminus or internally, via an amino acid side chain of the Fc protein. For example, an antibody comprising two IL-2 moieties fused to the light chains or heavy chains, respectively, may be used.

The Fc molecule may be an Fc molecule having a native Fc sequence or a genetically engineered Fc molecule.

The IL-2 portion of the IL-2 variant used in the present invention may be prepared recombinantly or synthetically. A nucleotide sequence and an amino acid sequence of wild type human IL-2 are known to a skilled person and are disclosed, for instance, in Genbank Entrez Reference 3558, as updated on updated on 3 Jun. 2018, and UniProt Reference UniProtKB—P60568, as last modified on May 23, 2018, respectively. The human IL-2 portion of the hIL-2 variant used in the present invention is preferably a mature IL-2, lacking the signal peptide. The human IL-2 portion of the hIL-2 variant used in the present invention may be glycosylated or unglycosylated. A preferred human IL-2 portion of the hIL-2 variant which can be used in the present invention is commercially available, including for pharmaceutical uses, and it is authorized for use in human patients as aldesleukin. Aldesleukin is a recombinant unglycosylated des-alanyl-1, serine-125 human interleukin-2, recombinantly produced in E. coli. Roncoleukin® is a recombinant human IL-2 produced in yeast.

In a preferred embodiment, the human IL-2 portion of the hIL-2 variant used in the present invention is aldesleukin, Roncoleukin®, or wildtype IL-2. Aldesleukin is the active ingredient of Proleukin®. Aldesleukin is an unglycosylated variant of mature human IL-2 comprising two amino acid modifications as compared to the sequence of mature human IL-2: the deletion of the first amino acid (alanine) and the substitution of cysteine at position 125 by serine.

Mature hIL-2 protein has a molecular weight of about 15 kDa.

In general, it is preferred to continue the at least once daily or continuous administration for subsequent days as long as the side effects of the IL-2 variant are tolerated or are acceptable for the patient. In case the side of effects of the IL-2 variant are tolerated or are acceptable for the patient, the at least once daily or continuous administration of the IL-2 variant may be performed for up to 10, 15, 20 or 21 days or even longer.

Therefore, in another preferred embodiment of the present invention, the IL-2 variant is administered for at least 3 or 4 subsequent days, and/or is administered for between 2 and 21 subsequent days. Preferably, the IL-2 variant is administered for at least 3, 4, 5, 6, 7, 8, 9 or 10 subsequent days. In another preferred embodiment, the IL-2 variant is administered for between 3 and 21, 4 and 21, 5 and 21, 3 and 15, 4 and 15, 5 and 15, 30 and 10, 4 and 10 or 5 and 10 subsequent days, such as for 2, 3, 4, 5, 6, 7, 8, 9, 10, 11, 12, 13, 14, 15, 16, 17, 18, 19, 20 or 21 subsequent days.

The administration of the IL-2 variant of the embodiments of the inventions begins within 5 days after the administration of the class I MHC expressing, antigen presenting cells. It is preferred that administration of the IL-2 variant begins at the day of the administration of the class I MHC expressing, antigen presenting cells or within 1 day, 2 days, 3 days or 4 days after said day.

The IL-2 variant may be administered by various routes. In a preferred embodiment of the present invention, the IL-2 variant is administered systemically, locally, intravenously, subcutaneously, intraperitoneally, intratumorally, peritumorally or intradermally, more preferably by local, intravenous, subcutaneous, intraperitoneal, intratumoral, peritumoral or intradermal injection.

In another preferred embodiment of the present invention, the IL-2 variant is administered at a dose of from 10 to 400 μg IL-2/kg BW per day. For example, the IL-2 variant is administered at a dose of from 10, 20, 30, 40 or 50 to 100, 200, 300, 400 or 800 μg IL-2 variant/kg BW per day.

Further, the IL-2 variant may be provided in a formulation as known to skilled persons, depending on the route of administration. For example, when intended for injection, such as e.g. intravenous or intraperitoneal injection, a solution or a dry, dried or lyophilized IL-2 variant may be provided. A dry, dried or lyophilized IL-2 variant may be reconstituted prior to use. The solution is preferably an aqueous solution, such as an aqueous buffered saline solution. The formulation comprising the IL-2 variant preferably contains one or more pharmaceutically acceptable excipients, such as water, buffering agents, such as monobasic and/or dibasic sodium phosphate, and/or mannitol. Pharmaceutically acceptable excipients are known to a skilled person.

In one embodiment IL-2 or variant thereof (especially with extended half-life such as IL-2cx) is administered repeatedly after the administration of the class I MHC expressing, antigen presenting cells in a time frame of from 0 h to 14 days after the administration of the class I MHC expressing, antigen presenting cells.

In one embodiment IL-2 (in one embodiment IL-2cx) is administered in a time frame of from 0 h to 9 days, in one embodiment from 0 h to 5 days after the administration of the class I MHC expressing, antigen presenting cells. It is preferred that administration of the IL-2 variant begins at the day of the administration of the class I MHC expressing, antigen presenting cells or within 1 day, 2 days, 3 days or 4 days after said day.

In a further aspect, the invention provides a method for treating a disease, selected from cancer and a viral infection.

The method for treating a disease of the invention comprises the steps of
amplifying a cellular cytotoxic immune response towards a virus-derived peptide, followed by
administering an effective amount of a peptide-MHC-I-antibody fusion protein, wherein the peptide-MHC-I-antibody fusion protein comprises
   (i) an antibody specifically binding to a target cell, and
   (ii) a polypeptide comprising in N- to C-terminal direction a virus-derived peptide, a β2-microglobulin and the extracellular domains α1, α2, and α3 of a class I MHC molecule, to a patient in need thereof.

In one embodiment the cellular cytotoxic immune response towards the virus-derived peptide has been induced by vaccination with an inducing medicament and/or by infection with a virus, thereby activating T cells against the virus-derived peptide; and/or the amplifying medicament comprises a class I MHC expressing, antigen presenting cell, wherein the MHC-I is bound to the virus-derived peptide, and a Th1 adjuvant, which is a danger signal.

In one embodiment the step of activating T cells against the virus-derived peptide of the method of inducing a cellular cytotoxic immune response towards the virus-derived peptide comprises administering an effective amount of an antibody-peptide fusion protein comprising
(a) an antibody or a variant or fragment thereof, specifically binding to chemokine (C motif) receptor 1 (XCR1), and
(b) the virus-derived peptide, wherein the peptide is covalently coupled to the antibody or a variant or fragment thereof.

In one embodiment the step of activating T cells against the virus-derived peptide of the method of inducing a cellular cytotoxic immune response towards the virus-derived peptide comprises administering an effective amount of an antibody-peptide fusion protein comprising
(a) lymphotactin (XCL1) or a variant or fragment thereof, specifically binding to XCR1, and
(b) the virus-derived peptide, wherein the peptide is covalently coupled to XCL1 or a variant or fragment thereof.

In one embodiment of the invention the inducing medicament is administered in combination with a Th1 adjuvant, which is a danger signal.

In another embodiment of the invention interleukin-2 (IL-2) or a IL-2 variant with extended half-life in the patient is administered after the administration of the class I MHC expressing, antigen presenting cell.

In still another embodiment of the invention the Th1 adjuvant is polyinosinic:polycytidylic acid (poly I:C).

Yet, in another embodiment of the invention the class I MHC expressing, antigen presenting cell, and the adjuvant is administered 3 days to 14 days, preferably 4 days to 10 days, after the T cells of patient were activated against said virus-derived peptide.

In another embodiment of the invention the therapeutic medicament is administered as long as the level of virus-derived peptide-specific CD8⁺ T cells is elevated as a result of the administration of the amplifying medicament, preferably wherein the therapeutic medicament is administered within 29 days, particularly 20 days, especially 14 days, from the administration of the amplifying medicament. More preferably, the therapeutic medicament is administered shortly after the amplification, such as within 3, 2 or 1 day from the administration of the amplifying medicament or immediately thereafter.

In still another embodiment of the invention the antibody comprised in the peptide-MHC-I-antibody fusion protein specifically binds to a target cell selected from the group of cancer cells and virus-infected cells.

In still another embodiment of the invention, the peptide-MHC-I-antibody fusion protein for use, or the kit of parts is for use in treating cancer or a viral infection.

In one embodiment the disease is cancer. In one embodiment the cancer is a non-solid tumor.

In one embodiment of the invention, wherein the treated disease is cancer, the cancer is selected from the group of lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, and lymphocytic leukemia. In one embodiment the cancer is selected from lymphomas, lymphocytic leukemia, bone cancer, and Hodgkin's Disease.

In one embodiment the disease is a viral infection. In one embodiment the disease is a chronic viral infection. In one embodiment the viral infection is selected from an infection with HBV, HCV, and HPV.

Further embodiments as listed above with respect to the medical use of the invention apply to the method of treatment as well.

### (c) Specific Embodiments of the Invention

In the following specific embodiments of the invention are listed.
1. A peptide-MHC-I-antibody fusion protein, wherein the peptide-MHC-I-antibody fusion protein comprises
   (i) an antibody specifically binding to a target cell, and
   (ii) a polypeptide comprising in N- to C-terminal direction a virus-derived peptide, a β2-microglobulin and the extracellular domains α1, α2, and α3 of a class I MHC molecule,
   for use as a therapeutic medicament,
   wherein the peptide-MHC-I-antibody fusion protein is to be administered to a patient, wherein a cellular cytotoxic immune response towards the virus-derived peptide has been induced in the patient and wherein the induced immune response towards the virus-derived peptide has been amplified in the patient by an amplifying medicament.
2. The peptide-MHC-I-antibody fusion protein for use according to embodiment 1,
   wherein the cellular cytotoxic immune response towards the virus-derived peptide is induced by vaccination comprising the steps of
   wherein the cellular cytotoxic immune response towards the virus-derived peptide has been induced in the patient by vaccination with an inducing medicament and/or by infection with a virus, thereby activating T cells against the virus-derived peptide.
3. The peptide-MHC-I-antibody fusion protein for use according to embodiment 1 or 2,
   wherein the amplifying medicament comprises a class I MHC expressing, antigen presenting cell, wherein the MHC-I is bound to the virus-derived peptide, and a Th1 adjuvant, which is a danger signal.
4. The peptide-MHC-I-antibody fusion protein for use according to embodiment 3, wherein the inducing medicament comprises
   (a) an antibody or a variant or fragment thereof, specifically binding to chemokine (C motif) receptor 1 (XCR1), and
   (b) the virus-derived peptide, wherein the peptide is covalently coupled to said antibody.
5. The peptide-MHC-I-antibody fusion protein for use according to embodiment 3,
   wherein the inducing medicament comprises
      (a) lymphotactin (XCL1) or a variant or fragment thereof, specifically binding to XCR1, and
      (b) the virus-derived peptide, wherein the peptide is covalently coupled to XCL1 or the variant or fragment thereof.
6. A kit of parts comprising an amplifying medicament and a therapeutic medicament,
   the amplifying medicament comprising
   (a) a class I MHC expressing, antigen presenting cell, wherein the MHC-I is bound to the virus-derived peptide, and
   (b) a Th1 adjuvant, which is a danger signal adjuvant, wherein the amplifying medicament amplifies an immune response towards a virus-derived peptide in a patient, and
   the therapeutic medicament comprising
   (i) an antibody specifically binding to a target cell, and
   (ii) a polypeptide comprising in N- to C-terminal direction the virus-derived peptide, a β2-microglobulin and the extracellular domains α1, α2, and α3 of a class I MHC molecule.
7. The kit of parts of embodiment 6 further comprising an inducing medicament inducing a cellular cytotoxic immune response towards the virus-derived peptide in the patient.
8. The kit of parts of embodiment 6 or 7, for use as a medicament wherein the therapeutic medicament is to be administered to a patient with amplified immune response towards the virus-derived peptide, wherein the amplifying medicament amplifies an immune response towards a virus-derived peptide in a patient and wherein the inducing medicament induces the cellular cytotoxic immune response towards the virus-derived peptide in the patient by vaccination, particularly wherein the inducing medicament is as defined in embodiment 4 or 5.
9. A kit of parts comprising an inducing medicament and an amplifying medicament,
   the inducing medicament being defined as in embodiment 4 or 5, and
   the amplifying medicament being defined as in embodiment 6 for use in a method of making a patient susceptible for a treatment with the peptide-MHC-I-antibody fusion protein of any one of embodiments 1 to 5.
10. The peptide-MHC-I-antibody fusion protein for use according to one of embodiments 2 to 5, or the kit of parts for use of embodiment 8 or 9, wherein the inducing medicament has been or is administered in combination with a Th1 adjuvant, which is a danger signal.
11. The peptide-MHC-I-antibody fusion protein for use according to one of embodiments 3 to 5 or 10, or the kit of parts for use of any one of embodiments 8 to 10, wherein interleukin-2 (IL-2) or IL-2 variant with extended half-life in the patient has been or is administered after the administration of the class I MHC expressing, antigen presenting cell.
12. The peptide-MHC-I-antibody fusion protein for use according to one of embodiments 3 to 5 or 10 to 11, or the kit of parts for use of any one of embodiments 8 or 10 to 11, wherein the Th1 adjuvant is polyinosinic:polycytidylic acid (poly I:C).
13. The peptide-MHC-I-antibody fusion protein for use according to one of embodiments 3 to 5 or 10 to 12, or the kit of parts for use of any one of embodiments 8 or 10 to 12, wherein the class I MHC expressing, antigen presenting cell, and the adjuvant has been or is administered 3 days to 14 days, preferably 4 days to 10 days, after the T cells of patient were induced against said virus-derived peptide.
14. The peptide-MHC-I-antibody fusion protein for use according to one of embodiments 1 to 5 or 10 to 13, or the kit of parts for use of any one of embodiments 8 or 10 to 13, wherein the therapeutic medicament is administered when the level of virus-derived peptide-specific CD8+ T cells is elevated as a result of the administration of the amplifying medicament, preferably wherein the therapeutic medicament is administered within 29 days, particularly 20 days, especially 14 days, from the administration of the amplifying medicament, more preferably wherein the therapeutic medicament is administered shortly after the amplification, such as within 3, 2 or 1 day from the administration of the amplifying medicament or immediately thereafter.
15. The peptide-MHC-I-antibody fusion protein for use according to one of embodiments 1 to 5 or 10 to 14, or the kit of parts for use of any one of embodiments 8 or 10 to 14, wherein the antibody comprised in the peptide-MHC-I-antibody fusion protein specifically binds to a target cell selected from the group of cancer cells and virus-infected cells.
16. The peptide-MHC-I-antibody fusion protein for use according to one of embodiments 1 to 5 or 10 to 15, or the kit of parts for use of any one of embodiments 8 or 10 to 15, for use in treating cancer or a viral infection.
17. The peptide-MHC-I-antibody fusion protein/kit of parts for use of one of the preceding embodiments, wherein the polypeptide of (ii) is fused with its C-terminus to the N-terminus of one heavy chain of the antibody of (i).
18. The peptide-MHC-I-antibody fusion protein/kit of parts for use of one of the preceding embodiments comprising a polypeptide comprising in N- to C-terminal direction a virus-derived peptide, a first peptide linker, a β2-microglobulin, a second peptide linker, and the extracellular domains α1, α2, and α3 of a class I MHC molecule.
19. The peptide-MHC-I-antibody fusion protein/kit of parts for use of one of the preceding embodiments, wherein the polypeptide of (ii) is fused with its C-terminus to the N-terminus of one heavy chain of the antibody of (i) via a third peptide linker.
20. The peptide-MHC-I-antibody fusion protein/kit of parts for use of one of the preceeding embodiments, wherein a disulfide bridge is formed between a cysteine residue of the first peptide linker and a cysteine residue of the extracellular domains α1, α2, and α3 of the class I MHC molecule.
21. The peptide-MHC-I-antibody fusion protein/kit of parts for use of one of the preceding embodiments, wherein the peptide-MHC-I-antibody fusion protein comprises exactly one polypeptide comprising in N- to C-terminal direction the virus-derived peptide, the β2-microglobulin and the extracellular domains α1, α2, and α3 of a class I MHC molecule.
22. The peptide-MHC-I-antibody fusion protein/kit of parts for use of one of the preceding embodiments, wherein the peptide-MHC-I-antibody fusion protein comprises one or two polypeptides comprising in N- to C-terminal direction the virus-derived peptide, the β2-microglobulin and the extracellular domains α1, α2, and α3 of a class I MHC molecule.
23. The peptide-MHC-I-antibody fusion protein/kit of parts for use of one of the preceding embodiments, wherein the peptide-MHC-I-antibody fusion protein comprises two polypeptides comprising in N- to C-terminal direction the virus-derived peptide, the β2-microglobulin and the extracellular domains α1, α2, and α3 of a class I MHC molecule.
24. The peptide-MHC-I-antibody fusion protein/kit of parts for use of one of the preceding embodiments, wherein the peptide-MHC-I-antibody fusion protein comprises a peptide from cytomegalovirus (huCMV).
25. The peptide-MHC-I-antibody fusion protein/kit of parts for use of one of the preceding embodiments, wherein the virus-derived peptide consists of 5 to 15 amino acids.
26. The peptide-MHC-I-antibody fusion protein/kit of parts for use of one of the preceding embodiments, wherein the antibody of (i) is a full length antibody.
27. The peptide-MHC-I-antibody fusion protein/kit of parts for use of one of the preceding embodiments, wherein the antibody of (i) is of IgG or IgE class.
28. The peptide-MHC-I-antibody fusion protein/kit of parts for use of one of the preceding embodiments, wherein the antibody of (i) is of IgG1, IgG2, IgG3 or IgG4 isotype.
29. The peptide-MHC-I-antibody fusion protein/kit of parts for use of one of the preceding embodiments, wherein the antibody of (i) is of human IgG1 or human IgG2 isotype.
30. The peptide-MHC-I-antibody fusion protein/kit of parts for use of one of the preceding embodiments, wherein the antibody of (i) is of human IgG1 or human IgG2 isotype with the mutations L234A and L235A (numbering according to the EU index of Kabat).
31. The peptide-MHC-I-antibody fusion protein/kit of parts n for use of one of the preceding embodiments, wherein the antibody of (i) is of human IgG1 or human IgG2 isotype with the mutations D265A and N297A (numbering according to the EU index of Kabat).
32. The peptide-MHC-I-antibody fusion protein/kit of parts for use of one of the preceding embodiments, wherein the antibody of (i) is of human IgG1 or human IgG2 isotype with the mutation P329G (numbering according to the EU index of Kabat).
33. The peptide-MHC-I-antibody fusion protein/kit of parts for use of one of the preceding embodiments, wherein the antibody of (i) is of human IgG1 or human IgG2 isotype with the mutations L234A, L235A and P329G.
34. The peptide-MHC-I-antibody fusion protein/kit of parts for use of one of the preceding embodiments (i.e. when the peptide-MHC-I-antibody fusion protein comprises exactly one MHC polypeptide), wherein the antibody of (i) comprises amino acid mutations in the CH3 domains that support heterodimerization of the antibody heavy chains.
35. The peptide-MHC-I-antibody fusion protein/kit of parts for use of embodiment 34, wherein the antibody of (i) comprises up to five mutations in each CH3 domain that support heterodimerization of the antibody heavy chains.
36. The peptide-MHC-I-antibody fusion protein/kit of parts for use of embodiment 34 or 35, wherein in the tertiary structure of the antibody of (i) the CH3 domain of a first heavy chain and the CH3 domain of a second heavy chain form an interface that is located between the respective antibody CH3 domains, wherein the respective amino acid sequences of the CH3 domain of the first heavy chain and the CH3 domain of the second heavy chain each comprise a set of amino acids that is located within said interface in the tertiary structure of the antibody,
   (a) wherein from the set of amino acids that is located in the interface in the CH3 domain of one heavy chain at least one amino acid residue is substituted by an amino acid residue having a larger side chain volume than the original amino acid residue, thereby generating a protuberance within the interface, wherein the protuberance is located in the CH3 domain of the one heavy chain, and wherein the protuberance is positionable in a cavity located in the CH3 domain of the other heavy chain within the interface; and
   (b) wherein from the set of amino acids that is located in the interface in the CH3 domain of the other heavy chain at least one amino acid residue is substituted by an amino acid residue having a smaller side chain volume than the original amino acid residue, thereby generating a cavity within the interface, wherein the cavity is located in the CH3 domain of the other heavy chain, and wherein in the cavity the protuberance within the interface located in the CH3 domain of the one heavy chain is positionable.
37. The peptide-MHC-I-antibody fusion protein/kit of parts for use of embodiment 36, wherein said amino acid residue having a larger side chain volume than the original amino acid residue is selected from R, F, Y and W; and wherein said amino acid residue having a smaller side chain volume than the original amino acid residue is selected from A, S, T and V.
38. The peptide-MHC-I-antibody fusion protein/kit of parts for use of one of embodiment 34 to 37, wherein in the CH3 domain of the one heavy chain the amino acid T at position 366 is substituted by W; and in the CH3 domain of the other heavy chain the amino acid T at position 366 is substituted by S, the amino acid L at position 368 is substituted by A and the amino acid Y at position 407 is substituted by V (numbering according to EU index of Kabat).
39. The peptide-MHC-I-antibody fusion protein/kit of parts for use of one of embodiments 34 to 38, wherein in the CH3 domain of the one heavy chain the amino acid T at position 366 is substituted by W, the amino acid R at position 409 is substituted by D, and the amino acid K at position 370 is substituted by E; and in the CH3 domain of the other heavy chain the amino acid T at position 366 is substituted by S, the amino acid L at position 368 is substituted by A and the amino acid Y at position 407 is substituted by V, the amino acid D at position 399 is substituted by K, and the amino acid E at position 357 is substituted by K (numbering according to EU index of Kabat).
40. The peptide-MHC-I-antibody fusion protein/kit of parts for use of one of embodiments 34 to 39, wherein from the set of amino acids that is located in the interface in the CH3 domain of the one heavy chain a first amino acid is substituted by cysteine; and from the set of amino acids that is located in the interface in the CH3 domain of the other heavy chain a second amino acid is substituted by cysteine, wherein the second amino acid is facing the first amino acid within the interface; such that a disulfide bridge between the CH3 domain of the one heavy chain and the CH3 domain of the other heavy chain can be formed via the introduced cysteine residues.
41. The peptide-MHC-I-antibody fusion protein/kit of parts for use embodiment 34 to 40, wherein in the CH3 domain of the one heavy chain either the amino acid E at position 356 or the amino acid S at position 354 is substituted by C, and in the CH3 domain of the other heavy chain the amino acid Y at position 349 is substituted by C (numbering according to EU index of Kabat).
42. The peptide-MHC-I-antibody fusion protein/kit of parts for use of one of the preceding embodiments, wherein the peptide-MHC-I-antibody fusion protein comprises a class I MHC molecule is selected from the group of HLA-A*0101, HLA-A*0201, HLA-A*0301, HLA-B*0702, HLA-B*0801, HLA-B*4402, HLA-C*0401, HLA-C*0501, HLA-C*0701, HLA-C*0702, HLA-A*0201, HLA-A*1101, HLA-A*2402, HLA-A*340101, HLA-B*1301, HLA-B*1521, HLA-B*5601, HLA B*5602, HLA-C*0102, HLA-C*0401, HLA-C*0403, HLA C*1502, HLA-A*0201, HLA-A*2402, HLA C*0202, HLA-C*0304, HLA-C*0401, HLA-C*0702, HLA-A*1101, HLA-A*2402, HLA B*1504, HLA-C*0102, HLA-C*0304, HLA-C*0702, and HLA C*0801.
43. The peptide-MHC-I-antibody fusion protein/kit of parts for use of one of embodiments 18 to 42, wherein the peptide linkers are glycine-serine linkers.
44. The peptide-MHC-I-antibody fusion protein/kit of parts for use of one of the preceding embodiments, wherein the antibody-peptide fusion protein comprises an antibody specifically binding to human XCR-1.
45. The peptide-MHC-I-antibody fusion protein/kit of parts for use of one of the preceding embodiments, wherein the disease is cancer or a viral infection.
46. A method for treating a cancer, wherein the method comprises the steps of
   amplifying a cellular cytotoxic immune response towards a virus-derived peptide, followed by
   administering an effective amount of a peptide-MHC-I-antibody fusion protein, wherein the peptide-MHC-I-antibody fusion protein comprises
   (i) an antibody specifically binding to a target cell, and
   (ii) a polypeptide comprising in N- to C-terminal direction a virus-derived peptide, a β2-microglobulin and the extracellular domains α1, α2, and α3 of a class I MHC molecule,
      to a patient in need thereof.
47. The method for treating a cancer of embodiment 46, wherein the cellular cytotoxic immune response towards the virus-derived peptide has been induced by vaccination with an inducing medicament and/or by infection with a virus, thereby activating T cells against the virus-derived peptide; and/or wherein the amplifying medicament comprises a class I MHC expressing, antigen presenting cell, wherein the MHC-I is bound to the virus-derived peptide, and a Th1 adjuvant, which is a danger signal.
48. The method for treating a cancer of embodiment 47, wherein the inducing medicament comprises
   (a) an antibody or a variant or fragment thereof, specifically binding to chemokine (C motif) receptor 1 (XCR1), and
   (b) the virus-derived peptide, wherein the peptide is covalently coupled to said antibody.
49. The method for treating a cancer of embodiment 47, wherein the inducing medicament comprises
   (a) lymphotactin (XCL1) or a variant or fragment thereof, specifically binding to XCR1, and
   (b) the virus-derived peptide, wherein the peptide is covalently coupled to XCL1 or the variant or fragment thereof.
50. A method for treating a viral infection,
   amplifying a cellular cytotoxic immune response towards a virus-derived peptide, followed by
   administering an effective amount of a peptide-MHC-I-antibody fusion protein, wherein the peptide-MHC-I-antibody fusion protein comprises
   (iii) an antibody specifically binding to a target cell, and
   (iv) a polypeptide comprising in N- to C-terminal direction a virus-derived peptide, a β2-microglobulin and the extracellular domains α1, α2, and α3 of a class I MHC molecule,
      to a patient in need thereof.
51. The method for treating a viral infection of embodiment 50,
   wherein the cellular cytotoxic immune response towards the virus-derived peptide has been induced by vaccination with an inducing medicament and/or by infection with a virus, thereby activating T cells against the virus-derived peptide; and/or wherein the amplifying medicament comprises a class I MHC expressing, antigen presenting cell, wherein the MHC-I is bound to the virus-derived peptide, and a Th1 adjuvant, which is a danger signal.
52. The method for treating a viral infection of embodiment 50, wherein the step of activating T cells against the virus-derived peptide of the method of inducing a cellular cytotoxic immune response towards the virus-derived peptide comprises administering an effective amount of an antibody-peptide fusion protein comprising wherein the inducing medicament comprises
   (a) an antibody or a variant or fragment thereof, specifically binding to chemokine (C motif) receptor 1 (XCR1), and
   (b) the virus-derived peptide, wherein the peptide is covalently coupled to said antibody.
53. The method for treating a viral infection of embodiment 50, wherein the inducing medicament comprises
   (a) lymphotactin (XCL1) or a variant or fragment thereof, specifically binding to XCR1, and
   (b) the virus-derived peptide, wherein the peptide is covalently coupled to XCL1 or the variant or fragment thereof.
54. Use of a peptide-MHC-I-antibody fusion protein, wherein the peptide-MHC-I-antibody fusion protein comprises
   (i) an antibody specifically binding to a target cell, and
   (ii) a polypeptide comprising in N- to C-terminal direction a virus-derived peptide, a β2-microglobulin and the extracellular domains α1, α2, and α3 of a class I MHC molecule,
   for the manufacture of a medicament for treating cancer or a viral infection,
   wherein the peptide-MHC-I-antibody fusion protein is to be administered to a patient,
   wherein a cellular cytotoxic immune response towards the virus-derived peptide has been induced in the patient and wherein the induced immune response towards the virus-derived peptide has been amplified in the patient by an amplifying medicament.
55. The use of embodiment 54,
   wherein the cellular cytotoxic immune response towards the virus-derived peptide has been induced by vaccination with an inducing medicament and/or by infection with a virus, thereby activating T cells against the virus-derived peptide; and/or the amplifying medicament comprises a class I MHC expressing, antigen presenting cell, wherein the MHC-I is bound to the virus-derived peptide, and a Th1 adjuvant, which is a danger signal.
56. The use of embodiment 55,
   wherein the inducing medicament comprises
   (a) an antibody or a variant or fragment thereof, specifically binding to chemokine (C motif) receptor 1 (XCR1), and
   (b) the virus-derived peptide, wherein the peptide is covalently coupled to said antibody.
57. The use of embodiment 55,
   wherein the inducing medicament comprises
   (a) lymphotactin (XCL1) or a variant or fragment thereof, specifically binding to XCR1, and
   (b) the virus-derived peptide, wherein the peptide is covalently coupled to XCL1 or the variant or fragment thereof.
58. Use of an antibody-peptide fusion protein comprising
   (i) an antibody specifically binding to chemokine (C motif) receptor 1 (XCR1), and
   (ii) a virus-derived peptide, wherein the peptide is covalently coupled to said antibody, for the manufacture of a medicament for treating cancer or a viral infection,
   wherein the antibody-peptide fusion protein is administered in combination with an amplifying medicament comprising
      (a) a class I MHC expressing, antigen presenting cell, wherein the MHC-I is bound to the virus-derived peptide, and
      (b) a Th1 adjuvant, which is a danger signal adjuvant,
   wherein the amplifying medicament amplifies an immune response towards a virus-derived peptide in a patient.

DESCRIPTION OF THE AMINO ACID SEQUENCES

| | |
|---|---|
| SEQ ID NO: 1 | human XCR1 |
| | MESSGNPESTTFFYYDLQSQPCENQAWVFATLATTVLYCLVFLLSLVGNSLVLWVLVKY |
| | ESLESLTNIFILNLCLSDLVFACLLPVWISPYHWGWVLGDFLCKLLNMIFSISLYSSIF |
| | FLTIMTIHRYLSWSPLSTLRVPTLRCRVLVTMAVWVASILSSILDTIFHKVLSSGCDYS |
| | ELTWYLTSVYQHNLFFLLSLGIILFCYVEILRTLFRSRSKRRHRTVKLIFAIVVAYFLS |
| | WGPYNFTLFLQTLFRTQIIRSCEAKQQLEYALLICRNLAFSHCCFNPVLYVFVGVKFRT |
| | HLKHVLRQFWFCRLQAPSPASIPHSPGAFAYEGASFY |
| SEQ ID NO: 2 | human IL-2 |
| | APTSSSTKKTQLQLEHLLLDLQMILNGINNYKNPKLTRMLTEKEYMPKKATEL |
| | KHLQCLEEELKPLEEVLNLAQSKNEHLRPRDLISNINVIVLELKGSETTFMCE |
| | YADETATIVEFLNRWITFCQSIISTLT |
| SEQ ID NO: 3 | N-terminal signal peptide of unprocessed human IL-2 |
| | MYRMQLLSCIALSLALVTNS |
| SEQ ID NO: 4 | virus-derived peptide |
| | NLVPMVATV |
| SEQ ID NO: 5 | virus-derived peptide |
| | VLEETSVML |
| SEQ ID NO: 6 | virus-derived peptide |
| | NLVPMVATV |
| SEQ ID NO: 7 | virus-derived peptide |
| | RIFAELEGV |
| SEQ ID NO: 8 | virus-derived peptide |
| | IIYTRNHEV |
| SEQ ID NO: 9 | virus-derived peptide |
| | VLAELVKQI |
| SEQ ID NO: 10 | virus-derived peptide |
| | AVGGAVASV |
| SEQ ID NO: 11 | virus-derived peptide |
| | TVRSHCVSK |
| SEQ ID NO: 12 | virus-derived peptide |
| | IMREFNSYK |
| SEQ ID NO: 13 | virus-derived peptide |
| | GPISHGHVLK |
| SEQ ID NO: 14 | virus-derived peptide |
| | ATVQGQNLK |
| SEQ ID NO: 15 | virus-derived peptide |
| | VYALPLKML |
| SEQ ID NO: 16 | virus-derived peptide |
| | AYAQKIFKIL |
| SEQ ID NO: 17 | virus-derived peptide |
| | QYDPVAALF |
| SEQ ID NO: 18 | virus-derived peptide |
| | YVKVYLESF |
| SEQ ID NO: 19 | virus-derived peptide |
| | DIYRIFAEL |
| SEQ ID NO: 20 | virus-derived peptide |
| | VFETSGGLVV |
| SEQ ID NO: 21 | virus-derived peptide |
| | KARDHLAVL |
| SEQ ID NO: 22 | virus-derived peptide |
| | QARLTVSGL |
| SEQ ID NO: 23 | virus-derived peptide |
| | KARAKKDEL |
| SEQ ID NO: 24 | virus-derived peptide |
| | QIKVRVDMV |
| SEQ ID NO: 25 | virus-derived peptide |
| | RRRHRQDAL |
| SEQ ID NO: 26 | virus-derived peptide |
| | ARVYEIKCR |
| SEQ ID NO: 27 | virus-derived peptide |
| | KMQVIGDQY |
| SEQ ID NO: 28 | virus-derived peptide |
| | NVRRSWEEL |
| SEQ ID NO: 29 | virus-derived peptide |
| | CPSQEPMSIYVY |
| SEQ ID NO: 30 | virus-derived peptide |
| | KPGKISHIMLDVA |
| SEQ ID NO: 31 | virus-derived peptide |
| | ELRRKMMYM |
| SEQ ID NO: 32 | virus-derived peptide |
| | IPSINVHHY |
| SEQ ID NO: 33 | virus-derived peptide |
| | FEQPTETPP |
| SEQ ID NO: 34 | virus-derived peptide |
| | YAYIYTTYL |
| SEQ ID NO: 35 | virus-derived peptide |
| | QEFFWDANDIY |
| SEQ ID NO: 36 | virus-derived peptide |
| | YEQHKITSY |
| SEQ ID NO: 37 | virus-derived peptide |
| | QEPMSIYVY |
| SEQ ID NO: 38 | virus-derived peptide |
| | SEHPTFTSQY |
| SEQ ID NO: 39 | virus-derived peptide |
| | QAIRETVEL |
| SEQ ID NO: 40 | virus-derived peptide |
| | TRATKMQVI |
| SEQ ID NO: 41 | virus-derived peptide |
| | DALPGPCI |
| SEQ ID NO: 42 | virus-derived peptide |
| | CEDVPSGKL |
| SEQ ID NO: 43 | virus-derived peptide |
| | HERNGFTVL |
| SEQ ID NO: 44 | virus-derived peptide |
| | PTFTSQYRIQGKL |
| SEQ ID NO: 45 | virus-derived peptide |
| | QMWQARLTV |
| SEQ ID NO: 46 | virus-derived peptide |
| | HELLVLVKKAQL |
| SEQ ID NO: 47 | virus-derived peptide |
| | DDYSNTHSTRYV |
| SEQ ID NO: 48 | virus-derived peptide |
| | SLYNTVATL |
| SEQ ID NO: 49 | virus-derived peptide |
| | GLCTLVAML |
| SEQ ID NO: 50 | virus-derived peptide |
| | GILGFVFTL |
| SEQ ID NO: 51 | virus-derived peptide |
| | STNRQSGRQ |
| SEQ ID NO: 52 | virus-derived peptide |
| | LLFGYPVYV |
| SEQ ID NO: 53 | virus-derived peptide |
| | FAEGFVRAL |
| SEQ ID NO: 54 | virus-derived peptide |
| | LIVIGILIL |
| SEQ ID NO: 55 | virus-derived peptide |
| | ILHTPGCV |
| SEQ ID NO: 56 | virus-derived peptide |
| | WYAQIQPHW |
| SEQ ID NO: 57 | virus-derived peptide |
| | AFSGVSWTM |
| SEQ ID NO: 58 | virus-derived peptide |
| | ILIGVVITW |
| SEQ ID NO: 59 | virus-derived peptide |
| | MMIPTVVAF |
| SEQ ID NO: 60 | virus-derived peptide |
| | PFPQSNAPI |
| SEQ ID NO: 61 | virus-derived peptide |
| | LLLTLLATV |
| SEQ ID NO: 62 | virus-derived peptide |
| | IVLEHGSCV |
| SEQ ID NO: 63 | virus-derived peptide |
| | LLFKTENGV |
| SEQ ID NO: 64 | virus-derived peptide |
| | PLNEAIMAV |
| SEQ ID NO: 65 | virus-derived peptide |
| | NLVRLQSGV |
| SEQ ID NO: 66 | virus-derived peptide |
| | LVISGLFPV |
| SEQ ID NO: 67 | virus-derived peptide |
| | LLLVAHYAI |
| SEQ ID NO: 68 | virus-derived peptide |
| | LALLAAFKV |
| SEQ ID NO: 69 | virus-derived peptide |
| | VILAGPMPV |
| SEQ ID NO: 70 | virus-derived peptide |
| | HVLGRLITV |
| SEQ ID NO: 71 | virus-derived peptide |
| | VTEHDTLLY |
| SEQ ID NO: 72 | virus-derived peptide |
| | NTDFRVLEL |
| SEQ ID NO: 73 | virus-derived peptide |
| | CVETMCNEY |
| SEQ ID NO: 74 | virus-derived peptide |
| | SS PPMFRV |
| SEQ ID NO: 75 | control peptide |
| | RALEYKNL |
| SEQ ID NO: 76 | m38-pMHC-I-anti-FAP-IgG heavy chain fused to peptide-MHC-I |
| | (m38 peptide underlined) |
| | MGWSCIILFLVATATGVHSSSPPMFRVGCGGSGGGGSGGGGSIQKTPQIQ |
| | VYSRHPPENGKPNILNCYVTQFHPPHIEIQMLKNGKKIPKVEMSDMSFSK |
| | DWSFYILAHTEFTPTETDTYACRVKHASMAEPKTVYWDRDMGGGGSGGGG |
| | SGGGGSGGGGSGPHSLRYFVTAVSRPGLGEPRYMEVGYVDDTEFVRFDSD |
| | AENPRYEPRARWMEQEGPEYWERETQKAKGNEQSFRVDLRTLLGCYNQSK |
| | GGSHTIQVISGCEVGSDGRLLRGYQQYAYDGCDYIALNEDLKTWTAADMA |
| | ALITKHKWEQAGEAERLRAYLEGTCVEWLRRYLKNGNATLLRTDSPKAHV |
| | THHSRPEDKVTLRCWALGFYPADITLTWQLNGEELIQDMELVETRPAGDG |
| | TFQKWASVVVPLGKEQYYTCHVYHQGLPEPLTLRWGSEVQLLESGGGLVQ |
| | PGGSLRLSCAASGFTFSSHAMSWVRQAPGKGLEWVSAIWASGEQYYADSV |
| | KGRFTISRDNSKNTLYLQMNSLRAEDTAVYYCAKGWLGNFDYWGQGTLVT |
| | VSSAKTTAPSVYPLAPVCGGTTGSSVTLGCLVKGYFPEPVTLTWNSGSLS |
| | SGVHTFPALLQSGLYTLSSSVTVTSNTWPSQTITCNVAHPASSTKVDKKI |
| | EPRVPITQNPCPPLKECPPCAAPDAAGGPSVFIFPPKIKDVLMISLSPMV |
| | TCVVVDVSEDAPDVQISWFVNNVEVHTAQTQTHREDYNSTLRVVSALPIQ |
| | HQDWMSGKEFKCKVNNRALGSPIEKTISKPRGPVRAPQVCVLPPPAEEMT |
| | KKEFSLSCAITGFLPAEIAVDWTSNGRTEQNYKNTATVLDSDGSYFMVSK |
| | LRVQKSTWERGSLFACSVVHEGLHNHLTTKTISRSLGK |
| SEQ ID NO: 77 | IE3-pMHC-I-anti-FAP-IgG heavy chain fused to peptide-MHC-I |
| | (IE3 peptide underlined) |
| | MGWSCIILFLVATATGVHSRALEYKNLGCGGSGGGGSGGGGSIQKTPQIQ |
| | VYSRHPPENGKPNILNCYVTQFHPPHIEIQMLKNGKKIPKVEMSDMSFSK |
| | DWSFYILAHTEFTPTETDTYACRVKHASMAEPKTVYWDRDMGGGGSGGGG |
| | SGGGGSGGGGSGPHSLRYFVTAVSRPGLGEPRYMEVGYVDDTEFVRFDSD |
| | AENPRYEPRARWMEQEGPEYWERETQKAKGNEQSFRVDLRTLLGCYNQSK |
| | GGSHTIQVISGCEVGSDGRLLRGYQQYAYDGCDYIALNEDLKTWTAADMA |
| | ALITKHKWEQAGEAERLRAYLEGTCVEWLRRYLKNGNATLLRTDSPKAHV |
| | THHSRPEDKVTLRCWALGFYPADITLTWQLNGEELIQDMELVETRPAGDG |
| | TFQKWASVVVPLGKEQYYTCHVYHQGLPEPLTLRWGSEVQLLESGGGLVQ |
| | PGGSLRLSCAASGFTFSSHAMSWVRQAPGKGLEWVSAIWASGEQYYADSV |
| | KGRFTISRDNSKNTLYLQMNSLRAEDTAVYYCAKGWLGNFDYWGQGTLVT |
| | VSSAKTTAPSVYPLAPVCGGTTGSSVTLGCLVKGYFPEPVTLTWNSGSLS |
| | SGVHTFPALLQSGLYTLSSSVTVTSNTWPSQTITCNVAHPASSTKVDKKI |
| | EPRVPITQNPCPPLKECPPCAAPDAAGGPSVFIFPPKIKDVLMISLSPMV |
| | TCVVVDVSEDAPDVQISWFVNNVEVHTAQTQTHREDYNSTLRVVSALPIQ |
| | HQDWMSGKEFKCKVNNRALGSPIEKTISKPRGPVRAPQVCVLPPPAEEMT |
| | KKEFSLSCAITGFLPAEIAVDWTSNGRTEQNYKNTATVLDSDGSYFMVSK |
| | LRVQKSTWERGSLFACSVVHEGLHNHLTTKTISRSLGK |
| SEQ ID NO: 78 | anti-FAP-IgG heavy chain |
| | MGWSCIILFLVATATGVHSEVQLLESGGGLVQPGGSLRLSCAASGFTFSS |
| | HAMSWVRQAPGKGLEWVSAIWASGEQYYADSVKGRFTISRDNSKNTLYLQ |
| | MNSLRAEDTAVYYCAKGWLGNFDYWGQGTLVTVSSAKTTAPSVYPLAPVC |
| | GGTTGSSVTLGCLVKGYFPEPVTLTWNSGSLSSGVHTFPALLQSGLYTLS |
| | SSVTVTSNTWPSQTITCNVAHPASSTKVDKKIEPRVPITQNPCPPLKECP |
| | PCAAPDAAGGPSVFIFPPKIKDVLMISLSPMVTCVVVDVSEDAPDVQISW |
| | FVNNVEVHTAQTQTHREDYNSTLRVVSALPIQHQDWMSGKEFKCKVNNRA |
| | LGSPIEKTISKPRGPVRAPQVYVLPPCAEEMTKKEFSLWCMITGFLPAEI |
| | AVDWTSNGRTEQNYKNTATVLDSDGSYFMYSKLRVQKSTWERGSLFACSV |
| | VHEGLHNRFTTKTISRSLGK |
| SEQ ID NO: 79 | anti-FAP-IgG light chain |
| | MGWSCIILFLVATATGVHSEIVLTQSPGTLSLSPGERATLSCRASQSVSR |
| | SYLAWYQQKPGQAPRLLIIGASTRATGIPDRFSGSGSGTDFTLTISRLEP |
| | EDFAVYYCQQGQVIPPTFGQGTKVEIK RADAAPTVSIFPPSSEQLTSGGA |
| | SVVCFLNNFYPKDINVKWKIDGSERQNGVLNSWTDQDSKDSTYSMSSTLT |
| | LTKDEYERHNSYTCEATHKTSTSPIVKSFNRNEC |
| SEQ ID NO: 80 | anti-FAP 28H1 VH |
| | MGWSCIILFLVATATGVHSEVQLLESGGGLVQPGGSLRLSCAASGFTFSS |
| | HAMSWVRQAPGKGLEWVSAIWASGEQYYADSVKGRFTISRDNSKNTLYLQ |
| | MNSLRAEDTAVYYCAKGWLGNFDYWGQGTLVTVSS |
| SEQ ID NO: 81 | anti-FAP 28H1 VL |
| | MGWSCIILFLVATATGVHSEIVLTQSPGTLSLSPGERATLSCRASQSVSR |
| | SYLAWYQQKPGQAPRLLIIGASTRATGIPDRFSGSGSGTDFTLTISRLEP |
| | EDFAVYYCQQGQVIPPTFGQGTKVE1K |
| SEQ ID NO: 82 | peptide linker |
| | GCGGSGGGGSGGGGS |
| SEQ ID NO: 83 | peptide linker |
| | GGGGSGGGGSGGGGSGGGGS |
| SEQ ID NO: 84 | anti-XCR1-IgG (MARX10) heavy chain with His Tag |
| | MGWSCIILFLVATATGVHSQVQLQQPGAELVKPGASVKLSCKASGYTFTN |
| | YWIHWMKQRPGQGLEWIGMIHPNSDNTKYNEKFKAKAILTVDKSSSTAYM |
| | QLSSLTSEDSAVYYCARFANDGAYWGQGTLVTVSAAKTTAPSVYPLAPVC |
| | GGTTGSSVTLGCLVKGYFPEPVTLTWNSGSLSSGVHTFPALLQSGLYTLS |
| | SSVTVTSNTWPSQTITCNVAHPASSTKVDKKIEPRVPITQNPCPPLKECP |
| | PCAAPDAAGGPSVFIFPPKIKDVLMISLSPMVTCVVVDVSEDAPDVQISW |
| | FVNNVEVHTAQTQTHREDYNSTLRVVSALPIQHQDWMSGKEFKCKVNNRA |
| | LGSPIEKTISKPRGPVRAPQVYVLPPPAEEMTKKEFSLTCMITGFLPAEI |
| | AVDWTSNGRTEQNYKNTATVLDSDGSYFMYSKLRVQKSTWERGSLFACSV |
| | VHEGLHNHLTTKTISRSLGGGGGSLPETGGSGSHHHHHH |
| SEQ ID NO: 85 | anti-XCR1-IgG (MARX10) light chain |
| | MGWSCIILFLVATATGVHSDVVVTQTPLSLPVSLGDPASISCKSSQSLVH |
| | SNGNTYLHWYLQKPGQSPKLLIYKISNRFSGVPDRFSGSGSGTDFTLKIS |
| | RVEAEDLGVYFCSQNTHVPYTFGGGTKLEIKRADAAPTVSIFPPSSEQLT |
| | SGGASVVCFLNNFYPKDINVKWKIDGSERQNGVLNSWTDQDSKDSTYSMS |
| | STLTLTKDEYERHNSYTCEATHKTSTSPIVKSFNRNEC |
| SEQ ID NO: 86 | peptide |
| | SIINFEKL |

### EXAMPLES

The following examples are provided to aid the understanding of the present invention, the true scope of which is set forth in the appended claims. It is understood that modifications can be made in the procedures set forth without departing from the scope of the invention.

### Materials & General Methods

### Cell Lines

The murine melanoma cell line B16-F10 (ATCC) and the murine colorectal cancer cell line MC38 (City of Hope National Medical Center) were stably transfected to express murine fibroblast activation protein (FAP). B16-F10 cells were grown in RPMI supplemented with 10% fetal bovine serum under selection with puromycin and MC38 cells were grown in DMEM supplemented with 10% fetal bovine serum, L-glutamine, sodium pyruvate, non-essential amino acids under selection with puromycin.

### Example 1

### Provision of Peptide-MHC-I-Antibody Fusion Protein Specifically Binding to Fibroblast Activating Protein (FAP)

For generation of an exemplary peptide-MHC-I-antibody fusion protein monoclonal antibody 28H1, which is a non-internalizing anti-fibroblast activating protein (FAP) antibody with high picomolar affinity for both mouse and human FAP was used to provide the binding site for targeting. A murinized version of the 28H1 was used to target engineered tumor cell lines expressing murine FAP. Amino acid sequences of the VH and VL domains of the 28H1 antibody are depicted in SEQ ID NO:80 and SEQ ID NO:81.

Two different peptide-MHC-I-antibody fusion proteins were generated:
(a) one fusion protein comprising the antigenic peptide mCMV-derived peptide m38 (sequence: SSPPMFRV, SEQ ID NO:74, the fusion protein is herein termed “m38-pMHC-I-anti-FAP-IgG”); and
(b) one fusion protein comprising a control nonameric peptide IE3 (sequence: RALEYKNL, SEQ ID NO:75, the control fusion protein is herein termed “IE3-pMHC-I-anti-FAP-IgG”).
   In brief, the peptide-MHC-I-antibody fusion proteins comprise from N- to C-terminal direction the respective peptide, a first linker (sequence: GCGGS(G₄S)₂, SEQ ID NO:82), murine β-2-microglobulin, a second linker (sequence: (G₄S)₄, SEQ ID NO:83), α1-3 domains of H-2Kb mouse MHC class I complex carrying a Y84C mutation), a third linker (sequence: GS) followed by the antibody heavy chain variable domain and an effector-free mutated IgG2c constant domain (a scheme of the molecule is provided in FIG. 1). The polypeptide comprising the peptide and the MHC class I domains was stabilized by an artificial disulfide bridge between the first linker (position 2 of the linker) and the MHC-heavy chain (position Y84C of the MHC class I heavy chain). The isotype of the antibody light chain was mouse kappa. The constant domains of the antibody heavy chains were mutated to remove all Fcγ-mediated effector functions (LALA-mutation: L234A, L235A and DAPG mutation: D270A, P329G). The peptide-MHC-I-antibody fusion proteins comprised one polypeptide comprising the peptide and the MHC class I domains, resulting in that the molecule comprises two different heavy chains. In order to facilitate heterodimerization of both heavy chains, knob-into-hole mutation were introduced in the CH3 domains. The heavy chain that was fused to the polypeptide comprising the peptide and the MHC class I domains carried the “hole” mutations: T366S, M368A, Y407V; the unfused heavy chain carried the “knob” mutation: T366W. To stabilize the heterodimerization of the two heavy chains an additional disulfide bridge was introduced between the two antibody heavy chains: Y349C for the “hole” and P354C for the “knob” antibody heavy chain.

The amino acid sequences of the three respective polypeptide chains of the two fusion proteins are indicated in the following table:

| | Peptide-MHC | | |
|---|---|---|---|
| | class I fused | Unfused | |
| | antibody heavy | antibody | Antibody light |
| | chain | heavy chain | chain |
| m38-pMHC-I- | SEQ ID NO: 76 | SEQ ID NO: 78 | SEQ ID NO: 79 |
| anti-FAP-IgG | | | |
| IE3-pMHC-I- | SEQ ID NO: 77 | SEQ ID NO: 78 | SEQ ID NO: 79 |
| anti-FAP-IgG | | | |

Protein production was performed as described before (Bacac M, Klein C, Umana P. CEA TCB: A novel head-to-tail 2:1 T cell bispecific antibody for treatment of CEA-positive solid tumors. Oncoimmunology. 2016; 5:e1203498; Schmittnaegel M, Hoffmann E, Imhof-Jung S, Fischer C, Drabner G, Georges G, et al. A New Class of Bifunctional Major Histocompatibility Class I Antibody Fusion Molecules to Redirect CD8 T Cells. Mol Cancer Ther. 2016; 15:2130-42). In brief, transient transfections in Hek293 cells were performed followed by protein A affinity and size-exclusion chromatography. Purified proteins were buffered in 20 mM Histidine and 140 mM sodium chloride at pH 6.0. Purified proteins were analyzed by SDS-PAGE, size exclusion chromatography and electrospray ionization mass spectrometry.

Functionality of the molecules was assessed as follows:

Mouse surrogate peptide-MHC-I-antibody fusion proteins were expressed with the expected yields, protein purity and quality. Flow cytometric analysis confirmed that antigen binding was maintained and that MHC class I complexes were successfully delivered to the target cell surface with (data not shown).

### Example 2

Provision of Antibody-Peptide Fusion Protein Comprising a mCMV-Derived Peptide and an Anti-XCR1 Antibody and Vaccination

Vaccination was performed by targeting of the immunodominant mCMV-peptide to XCR1+ dendritic cells, as described previously (Hartung E, Becker M, Bachem A, Reeg N, Jakel A, Hutloff A, et al. Induction of potent CD8 T cell cytotoxicity by specific targeting of antigen to cross-presenting dendritic cells in vivo via murine or human XCR1. J Immunol. 2015; 194:1069-79).

Previously described monoclonal antibody MARX10 that specifically binds to murine XCR1-expressing cells was used as the targeting antibody (WO2015140175, the MARX10 antibody used herein comprised a heavy chain amino acid sequence of SEQ ID NO:84, and a light chain amino acid sequence of SEQ ID NO:85). In order to provide the antibody-peptide fusion protein, the mCMV m38 peptide “SSPPMFRV”, SEQ ID NO:74 was fused to the C-terminus of the anti-XCR1 antibody MARX10 by enzymatic sortase coupling (Clancy K W, Melvin J A, McCafferty D G. Sortase transpeptidases: insights into mechanism, substrate specificity, and inhibition. Biopolymers. 2010; 94:385-96, Madej M P, Coia G, Williams C C, Caine J M, Pearce L A, Attwood R, et al. Engineering of an anti-epidermal growth factor receptor antibody to single chain format and labeling by Sortase A-mediated protein ligation. Biotechnol Bioeng. 2012; 109:1461-70). The antibody-peptide fusion protein comprising the m38 peptide and the MARX10 antibody is herein also referred to as “m38-MARX10”.

For vaccination, C57BL/6N mice were injected with 5 μg of m38-MARX10 together with 10 μg polyinosinic-polycytidylic acid (Poly(T:C), InvivoGen) i.v. as described previously (WO2015140175 and Hartung, E., et al., J Immunol, 2015. 194(3): p. 1069-79). Five days later, the peptide-specific CD8+ T cells were re-activated by injection of 10×10⁶ SSPPMFRV peptide-loaded splenocytes and 50 μg Poly(I:C) (InvivoGen) i.v. The following three days, mice were daily injected i.p. with antibody-complexed IL-2 (2.5 μg recombinant murine IL-2 and 10 μg antibody), which resulted in a further expansion of SSPPMFRV-specific CD8⁺ T cells (WO 2015/140175). For preparation of peptide-loaded cells, syngeneic splenocytes were incubated for 1-2 hours at 37° C. in a humidified incubator supplied with 5% CO₂ at a molar-peptide (SSPPMFRV) concentration of 10 μM. To remove unbound peptide, splenocytes were washed with PBS twice and then resuspended in PBS before injection into mice. Complexed IL-2 was prepared by reacting purified recombinant murine IL-2 (PeproTech) with anti-mouse IL-2 antibody JES6-5H4 (BioLegend) for 4 h at RT or overnight at 4° C. After vaccination and amplification, blood was drawn from mice to evaluate the CD8⁺ T cell response using flow cytometry.

The amount of m38-specific CD8+ T cells first increased to a peak of 15.0% on day 9 and then decreased constantly to about 4.4% between day 18 and 25 and finally went down to 2.5% in average on day 40. m38-H-2Kb-specific CD8+ T cells were CD44+/CD62L−/CD127+ effector cells. This vaccination protocol reached consistently levels of 1-4% m38-specific CD8+ T cells.

### Example 3

### CD8 T Cell Activation Mediated by Peptide-MHC-I-Antibody Fusion Protein Specifically Binding to FAP

The ability of peptide-MHC-I-antibody fusion protein (“pMHC-I-IgGs”) generated in Example 1 to activate CD8⁺ T cells was tested in an in vitro IFNγ activation assay.

Splenocytes from vaccinated mice (Example 2) were isolated and mixed in a 1:1 ratio with MC38 target cells expressing mFAP. The pMHC-I-IgGs (m38-pMHC-I-anti-FAP-IgG, and IE3-pMHC-I-anti-FAP-IgG control) were added and after six hours of incubation the CD8 T cells were analyzed by flow cytometry. As positive control m38 peptide-loaded tumor cell were used. After six hours of incubation the splenocytes were analyzed by flow cytometry for T cell markers and IFNγ production. Cells stained with fluorochrome-conjugated antibodies for detection of intracellular IFNγ.

Results are shown in FIG. 2. Frequency of IFNγ expressing CD8+ T cells measured in FACS upon incubation of MC38-mFAP tumor cells loaded with mCMV-derived m38 peptide (▾), mCMV m38-pMHC-I-anti-FAP-IgG (●) or mCMV IE3-pMHC-I-anti-FAP-IgG (▪) and unloaded tumor cells together with freshly isolated splenocytes (♦) from mCMV m38-vaccinated mice is illustrated. All graphs show mean of replicates (n=2) with error bars indicating standard deviation.

The m38-pMHC-I-anti-FAP-IgG could activate about 6% of all CD8+ T cells, which represents 10% of the m38-specific CD8+ T cell population. m38 peptide-loaded target cells mediated activation of about 12% of the entire CD8+ T cell population. The pMHC-I-IgG containing the control peptide IE3 mediated almost no IFNγ activation of CD8+ T cells indicating that activation of effector cells was strictly peptide-specific. Coincubation of splenocytes together with tumor cells in absence of effector molecules did not result in any IFNγ activation of CD8+ T cells either. In addition we found that peptide-loading of tumor cells achieved only at higher concentrations (5 nM) the maximum of IFNγ activation compared to pMHC-I-IgG treated tumor cells, which reached maximal IFNγ activation at lower concentrations (1 nM).

### Example 4

### In Vitro Assessment of Tumor Cell Elimination Mediated by Peptide-MHC-I-Antibody Fusion Protein Specifically Binding to FAP

In order to demonstrate that pMHC-I-IgGs are able to induce concentration-dependent and target-specific lysis of tumor cells, specific target cell elimination was tested in a cytotoxicity assay in the real-time cell analyzer xCELLigence (Roche) as described before (Schmittnaegel M, Levitsky V, Hoffmann E, Georges G, Mundigl O, Klein C, et al. Committing Cytomegalovirus-Specific CD8 T Cells to Eliminate Tumor Cells by Bifunctional Major Histocompatibility Class I Antibody Fusion Molecules. Cancer Immunol Res. 2015; 3:764-76).

In brief, the adherent tumor cell line MC38 recombinantly expressing mFAP was cultivated for 48 hours, splenocytes from m38-vaccinated mice were added at an effector cell:target cell ratio of 0.25:1. The pMHC-I-IgGs were added at different concentrations ranging from 0.1 to 100 nM. As positive control m38 peptide-loaded tumor cell were used. Kinetics of target cell lysis was measured in real time. Measurements were performed up to 48 hours in triplicates. Spontaneous release was measured from target cell with effector cells only. Specific lysis in percentage (%) is calculated as [(cell index spontaneous release−cell index specimen)/(cell index spontaneous release)]×100.

m38 peptide-loaded tumor cells were eliminated rapidly. The m38-pMHC-I-anti-FAP-IgG showed a delayed onset of cell killing as observed before (Schmittnaegel M, Levitsky V, Hoffmann E, Georges G, Mundigl O, Klein C, et al. Committing Cytomegalovirus-Specific CD8 T Cells to Eliminate Tumor Cells by Bifunctional Major Histocompatibility Class I Antibody Fusion Molecules. Cancer Immunol Res. 2015; 3:764-76). The IE3-pMHC-I-anti-FAP-IgG with the irrelevant control peptide for which there are no corresponding T cells did not trigger cell killing also not at high concentrations.

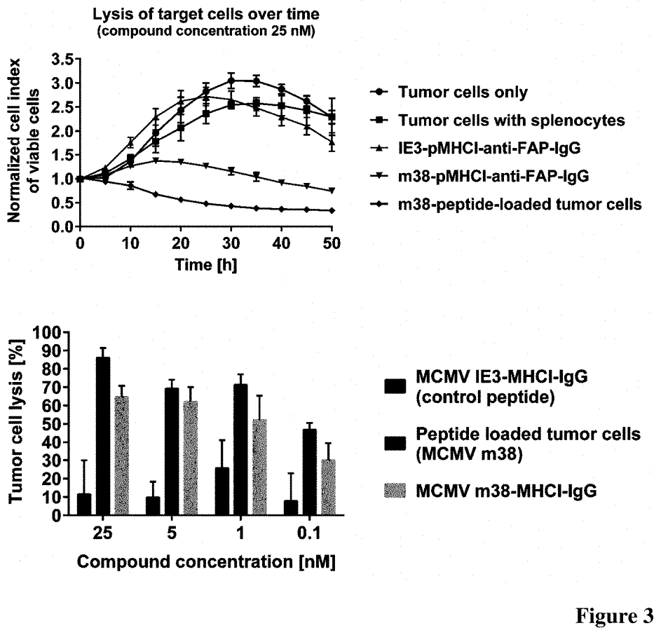

Results are shown in FIG. 3. Specific tumor cell lysis mediated by pMHC-I-IgGs after incubation with freshly isolated splenocytes from m38-vaccinated mice. Incubation of MC38-mFAP cells loaded with mCMV IE3-pMHC-I-anti-FAP-IgG (▴), mCMV m38 peptide(♦), or mCMV m38-pMHC-I-anti-FAP-IgG (▾) are illustrated. Lysis of target cells was measured over time. Kinetics of cell lysis at a compound concentration of 25 nM is shown (FIG. 3A). Tumor cell elimination after 40 hours is shown for all concentrations tested (FIG. 3B). All graphs show mean of replicates (n=3) with error bars indicating standard deviation.

### Example 5

### Inhibition of Formation of Lung Metastases Mediated by Peptide-MHC-I-Antibody Fusion Protein Specifically Binding to FAP (Preventive Setting)

In Examples 5 and 6, the therapeutic effect of pMHC-I-IgGs on the progression of lung tumors was assessed. Example 5 is displays a preventive setting, while Example 6 displays a therapeutic setting.

C57BL/6N mice were immunized to induce an endogenous CD8+ T cell response for the m38 epitope of mCMV.

For the in vivo assessment, 7- to 8-weeks-old C57BL/6N mice were obtained from Charles River Laboratories. To induce experimental lung metastasis (Fidler I J, Nicolson G L. Organ selectivity for implantation survival and growth of B16 melanoma variant tumor lines. J Natl Cancer Inst. 1976; 57:1199-202), mice were injected intravenously into the lateral tail vein with 2×10E5 mFAP-transfected B16 melanoma cells in a total volume of 100 μl (HBSS). For generation of effector cells, animals were immunized with the XCR1-targeted vaccination as described in Example 2. In the preventive setting used in this examples animals were pretreated with the therapeutic proteins 24 hours before tumor challenge followed by one therapeutic treatment three days later. Then mice were treated twice at an interval of 3 days.

More precisely, at the peak of the immune response with an average of 3.2% of m38-H-2Kb-specific CD8+ T cells in peripheral blood mice were treated intravenously with of 5 mg/kg pMHC-I-IgG or 2 mg/kg TCB. 24 hours after treatment mice were challenged intravenously with 2×10E5 mFAP-expressing B16-F10 melanoma cells. Two days after intravenous injection of tumor cells a second treatment with m38-pMHC-I-anti-FAP-IgG and anti-FAP-TCB molecules was administered.

Animals were monitored daily for general health condition. When stopping criterion (such as bad health condition, bodyweight loss >20%) was reached, mice were sacrificed.

On day 21 after injection of tumor cells the animals were sacrificed and the lungs were isolated. The metastatic burden was assessed by counting of visible metastases on the lung surface and quantification of TRP-2 expression by qPCR.

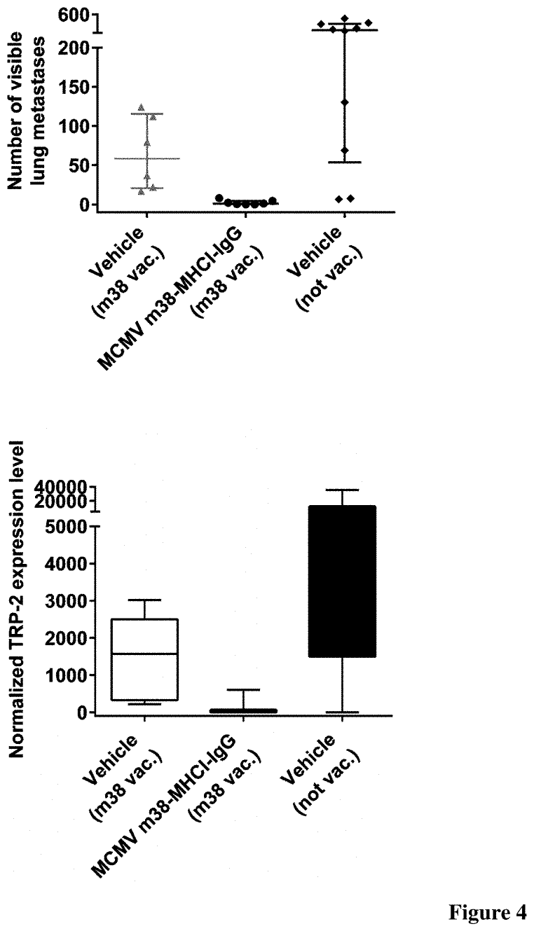

Results are shown in FIG. 4. Visible lung metastases were counted after harvest of lungs 21 days after injection of the tumor cells. MCMV m38-vaccinated (▴) and non-vaccinated (♦) mice were either treated twice with PBS, m38-pMHC-I-anti-FAP-IgG (●) or IE3-pMHC-I-anti-FAP-IgG (control) 24 hours before injection of tumor cells and two days after. All graphs show median of different animals per group (n=6-10) with error bars indicating interquartile range of counted lung metastasis (FIG. 4A) or qPCR quantification of TRP-2 in the lungs after 21 days of growth (FIG. 4B).

The treatment with m38-pMHC-I-anti-FAP-IgG eliminated almost all metastasis in all mice (number of metastasis 1 and 0, respectively and TRP-2 expression level 2 and 1, respectively).

### Example 6

### Inhibition of Tumor Spread by Therapeutic Treatment of Lung Metastases Mediated by Peptide-MHC-I-Antibody Fusion Protein Specifically Binding to FAP (Therapeutic Setting)

For assessment of the therapeutic effect of pMHC-I-IgGs on the progression of lung tumors in a therapeutic setting, a similar experimental setup as described for Example 5 was used. The difference was that in the therapeutic setting melanoma cells were allowed to grow in the lungs for nine days after intravenous injection.

In brief, M38-peptide immunized C57BL/6N mice were injected intravenously with 2×10E5 mFAP-transfected B16-F10 melanoma cells. After nine days, when lung metastases were already established, animals were treated twice at an interval of three days with 5 mg/kg pMHC-I-IgG during the CD8+ T cell peak with an average of 11.8% mCMV m38-H-2Kb-specific CD8+ T cells.

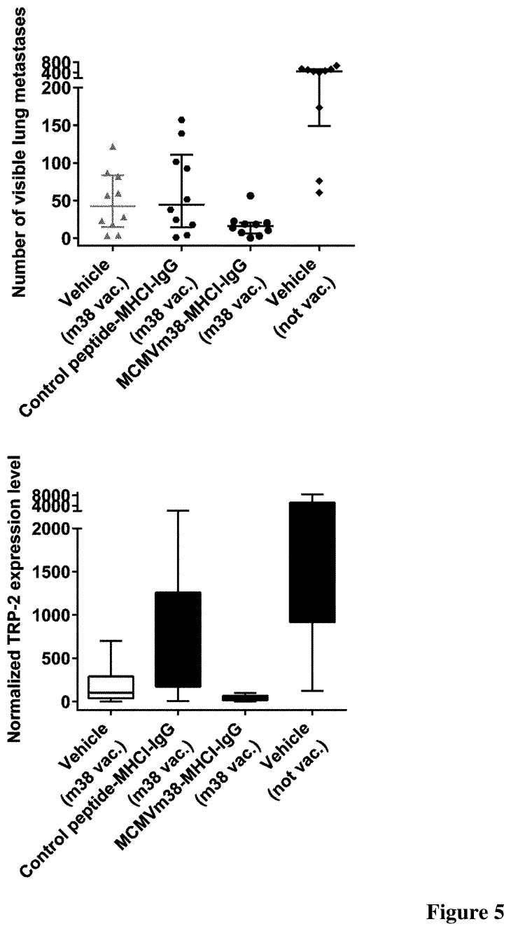

Results are shown in FIG. 5. Visible lung metastases were counted after harvest of lungs on day 21 of metastasis growth (FIG. 5A). TRP-2 expression was quantified by qPCR (FIG. 5B). MCMV m38-vaccinated (▴) and non-vaccinated mice (♦) were treated twice with either PBS, mCMV IE3-pMHC-I-anti-FAP-IgG control (●, left) or mCMV m38-pMHC-I-anti-FAP-IgG (●, right). Treatment started nine days after injection of B16-mFAP melanoma cells. All graphs show median of different animals per group (n=8-10) with error bars indicating interquartile range. All graphs show box plots with median of different animals per group (n=8-10) with whiskers indicating maximum and minimum value.

The therapeutic treatment of already established lung metastases with pMHC-I-IgG reduced the number of metastases. The vaccinated control group showed 43 metastases per lung (SEM: 12.5, STDEV 39.7) and a TRP-2 expression level of 103 (SEM: 278.0, STDEV 680.9) in median, while pMHC-I-IgG-treated lungs had 16 spots (SEM: 5.0, STDEV 15.7) and a TRP-2 expression level of 43 in median (SEM: 11.5, STDEV 30.4). The m38-vaccinated group treated with the pMHC-I-IgG containing the control peptide IE3 showed metastatic outgrowth of 45 (SEM: 17.8, STDEV 56.3) similar to the one of the vaccinated vehicle group indicating that tumor cell lysis was specifically mediated by recruitment of vaccination-induced, m38-H-2Kb-specific CD8⁺ T cells by means of pMHC-I-IgGs. The experiment demonstrated that with pMHC-I-IgG metastasis outgrowth can be delayed and reduced when B16-F10 tumor cells have already settled in the lung. It could be shown that antibody binding to the targeted tumor cells was maintained, because the tumor cells kept the expression of mFAP in vivo (data not shown).

1. A peptide-MHC-I-antibody fusion protein,
comprising
(i) an antibody specifically binding to a target cell, and
(ii) a polypeptide comprising in N- to C-terminal direction a virus-derived peptide, a β2-microglobulin and the extracellular domains α1, α2, and α3 of a class I MHC molecule,
as a therapeutic medicament,
wherein the peptide-MHC-I-antibody fusion protein is adapted to be administered to a patient, wherein a cellular cytotoxic immune response towards the virus-derived peptide has been induced in the patient and wherein the induced immune response towards the virus-derived peptide is capable of being amplified in the patient by an amplifying medicament.

2. The peptide-MHC-I-antibody fusion protein according to claim 1,
wherein the cellular cytotoxic immune response towards the virus-derived peptide has been induced in the patient by vaccination with an inducing medicament and/or by infection with a virus, thereby activating T cells against the virus-derived peptide.

3. The peptide-MHC-I-antibody fusion protein for use according to claim 1, wherein the amplifying medicament comprises a class I MHC expressing, antigen presenting cell, wherein the MHC-I is bound to the virus-derived peptide, and a Th1 adjuvant, which is a danger signal.

4. The peptide-MHC-I-antibody fusion protein according to claim 3,
wherein the inducing medicament comprises
(a) an antibody or a variant or fragment thereof, specifically binding to chemokine (C motif) receptor 1 (XCR1), and
(b) the virus-derived peptide, wherein the peptide is covalently coupled to said antibody.

5. The peptide-MHC-I-antibody fusion protein according to claim 3,
wherein the inducing medicament comprises
(a) lymphotactin (XCL1) or a variant or fragment thereof, specifically binding to XCR1, and
(b) the virus-derived peptide, wherein the peptide is covalently coupled to XCL1 or the variant or fragment thereof.

6. A kit of parts comprising an amplifying medicament and a therapeutic medicament,
the amplifying medicament comprising
(a) a class I MHC expressing, antigen presenting cell, wherein the MHC-I is bound to the virus-derived peptide, and
(b) a Th1 adjuvant, which is a danger signal adjuvant,
wherein the amplifying medicament amplifies an immune response towards a virus-derived peptide in a patient, and
the therapeutic medicament comprising
(i) an antibody specifically binding to a target cell, and
(ii) a polypeptide comprising in N- to C-terminal direction the virus-derived peptide, a β2-microglobulin and the extracellular domains α1, α2, and α3 of a class I MHC molecule.

7. The kit of parts of claim 6 further comprising an inducing medicament inducing a cellular cytotoxic immune response towards the virus-derived peptide in a patient.

8. The kit of parts of claim 6, wherein the therapeutic medicament is to be administered to a patient with amplified immune response towards the virus-derived peptide, wherein the amplifying medicament amplifies an immune response towards a virus-derived peptide in a patient and wherein the inducing medicament induces the cellular cytotoxic immune response towards the virus-derived peptide in the patient by vaccination, optionally wherein the inducing medicament comprises
(c) an antibody or a variant or fragment thereof, specifically binding to chemokine (C motif) receptor 1 (XCR1), and
(d) the virus-derived peptide, wherein the peptide is covalently coupled to said antibody.

9. A kit of parts comprising an inducing medicament and an amplifying medicament,
adapted for use in a method of making a patient susceptible for a treatment with the peptide-MHC-I-antibody fusion protein of claim 1.

10. The peptide-MHC-I-antibody fusion protein according to claim 2, wherein the inducing medicament is adapted to be administered in combination with a Th1 adjuvant, which is a danger signal.

11. The peptide-MHC-I-antibody fusion protein according to claim 3, wherein interleukin-2 (IL-2) or IL-2 variant with extended half-life in the patient has been or is capable of being administered after administration of the class I MHC expressing, antigen presenting cell.

12. The peptide-MHC-I-antibody fusion protein according to claim 3, wherein the Th1 adjuvant is polyinosinic:polycytidylic acid (poly I:C).

13. The peptide-MHC-I-antibody fusion protein according to claim 3, wherein the class I MHC expressing, antigen presenting cell, and the adjuvant are adapted to be administered 3 days to 14 days, optionally 4 days to 10 days, after the T cells of the patient were induced against said virus-derived peptide.

14. The peptide-MHC-I-antibody fusion protein according to claim 1, wherein the therapeutic medicament is adapted to be administered when the level of virus-derived peptide-specific CD8+ T cells is elevated as a result of the administration of the amplifying medicament, optionally wherein the therapeutic medicament is administered within 29 days, optionally 20 days, optionally 14 days, from administration of the amplifying medicament, optionally wherein the therapeutic medicament is administered shortly after the amplification, optionally within 3, 2 or 1 day from administration of the amplifying medicament or immediately thereafter.

15. The peptide-MHC-I-antibody fusion protein according to claim 1, wherein the antibody comprised in the peptide-MHC-I-antibody fusion protein specifically binds to a target cell selected from the group of cancer cells and virus-infected cells.

16. The peptide-MHC-I-antibody fusion protein according to claim 1 adapted for treating cancer or a viral infection.

17. A method for inducing an immune response in a patient comprising administering a peptide-MHC-I-antibody fusion protein comprising
i. an antibody specifically binding to a target cell, and
ii. a polypeptide comprising in N- to C-terminal direction a virus-derived peptide, a β2-microglobulin and the extracellular domains α1, α2, and α3 of a class I MHC molecule,
as a therapeutic medicament to said patient,
wherein a cellular cytotoxic immune response towards the virus-derived peptide is induced in the patient and wherein the induced immune response towards the virus-derived peptide is capable of being amplified in the patient by an amplifying medicament.

18. The method of claim 17, wherein the cellular cytotoxic immune response towards the virus-derived peptide is induced in the patient by vaccination with an inducing medicament and/or by infection with a virus, thereby activating T cells against the virus-derived peptide, and/or
wherein the amplifying medicament comprises a class I MHC expressing, antigen presenting cell, wherein the MHC-I is bound to the virus-derived peptide, and a Th1 adjuvant, which is a danger signal.

19. The method of claim 17, wherein the therapeutic medicament is administered when the level of virus-derived peptide-specific CD8+ T cells is elevated as a result of administration of the amplifying medicament, optionally wherein the therapeutic medicament is administered within 29 days, optionally 20 days, optionally 14 days, from administration of the amplifying medicament, optionally wherein the therapeutic medicament is administered shortly after the amplification, optionally within 3, 2 or 1 day from administration of the amplifying medicament or immediately thereafter, and/or
wherein the antibody comprised in the peptide-MHC-I-antibody fusion protein specifically binds to a target cell selected from the group of cancer cells and virus-infected cells.
